(19) United States
(12) Patent Application Publication
(10) Pub. No.: US 2011/0160300 A1
(43) Pub. Date: Jun. 30, 2011

(54) Renin Inhibitors and Methods of Use Thereof

(76) Inventors: Marlys Hammond, King of Prussia, PA (US); Patrick Stoy, King of Prussia, PA (US)

(21) Appl. No.: 12/999,969

(22) PCT Filed: Jun. 18, 2009
(86) PCT No.: PCT/US2009/003650

§ 371 Date: Mar. 10, 2011

Related U.S. Application Data
(60) Provisional application No. 61/074,271, filed on Jun. 20, 2008.

(51) Int. Cl. A61K 31/351; C07D 309/06; A61P 9/12; A61P 9/04; A61P 13/12; A61P 25/22; A61K 31/27; C07C 271/16

(52) U.S. Cl. 514/459; 549/426; 514/476; 560/27

(57) ABSTRACT

Disclosed are aspartic protease inhibitors represented by the following Formula: wherein R¹, R², R³, R⁴, R⁵, R⁶, R^{7a}, R^{7b} and n are as defined herein, or a pharmaceutically acceptable salt thereof, pharmaceutical compositions comprising the same and methods for treating an aspartic protease mediated disorder using the same.

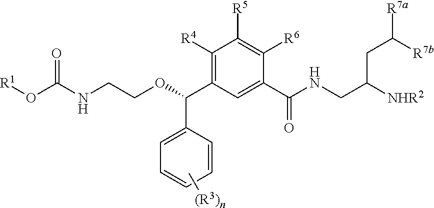

### RELATED APPLICATION

This application claims the benefit of U.S. Provisional Application No. 61/074,271, filed on Jun. 20, 2008.

The entire teachings of the above application are incorporated herein by reference.

### BACKGROUND OF THE INVENTION

Aspartic proteases, including renin, β-secretase (BACE), HIV protease, HTLV protease and plasmepsins I and II, are implicated in a number of disease states. In hypertension, elevated levels of angiotensin I, the product of renin catalyzed cleavage of angiotensinogen are present. Elevated levels of β amyloid, the product of BACE activity on amyloid precursor protein, are widely believed to be responsible for the amyloid plaques present in the brains of Alzheimer's disease patients. The viruses HIV and HTLV depend on their respective aspartic proteases for viral maturation. Plasmodium falciparum uses plasmepsins I and II to degrade hemoglobin.

In the renin-angiotensin-aldosterone system (RAAS), the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension,” in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J: Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of AT1 receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Il Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

### SUMMARY OF THE INVENTION

One embodiment of the invention is an aspartic protease inhibitor, which is a compound represented by Formula (I):

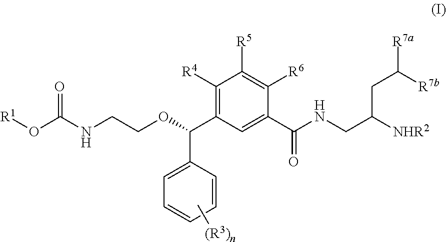

wherein:
R¹ is C₁-C₄ alkyl, C₃-C₆ cycloalkyl or C₃-C₆ cycloalkyl-C₁-C₄ alkyl-;
R² is H or C₁-C₄ alkyl;
each R³ is independently selected from F, Cl, Br, cyano, nitro, C₁-C₄ alkyl, C₁-C₄ haloalkyl, C₁-C₄ alkoxy, C₁-C₄ haloalkoxy, and C₁-C₄ alkylsulfonyl-;
n is 0, 1, 2, or 3;
R⁴, R⁵ and R⁶ are selected from H, halo and C₁-C₃ alkyl, wherein one of R⁴, R⁵ or R⁶ is H, halo or C₁-C₃ alkyl and the other two of R⁴, R⁵ and R⁶ are H; and
R^{7a} and R^{7b} are each independently C₁-C₃ alkyl, or R^{7a} and R^{7b} taken together with the carbon atom to which they are attached form a 5-6 membered carbocyclic or heterocylic ring, where the heterocyclic ring contains one oxygen atom;
   or a pharmaceutically acceptable salt thereof.

Another embodiment of the invention is a pharmaceutical composition comprising a pharmaceutically acceptable carrier or diluent and an aspartic protease inhibitor represented by Formula (I), or a pharmaceutically acceptable salt thereof. The pharmaceutical composition is used in therapy, e.g., for inhibiting an aspartic protease mediated disorder in a subject.

Another embodiment of the invention is a method of antagonizing one or more aspartic proteases in a subject in need of such treatment. The method comprises administering to the subject an effective amount of an aspartic protease inhibitor represented by Formula (I), or a pharmaceutically acceptable salt thereof.

Another embodiment of the invention is a method of treating an aspartic protease mediated disorder in a subject. The method comprises administering to the subject an effective amount of an aspartic protease inhibitor represented by Formula (I), or a pharmaceutically acceptable salt thereof.

Another embodiment of the invention is the use of an aspartic protease inhibitor represented by Formula (I), or a pharmaceutically acceptable salt thereof, for the manufacture of a medicament for antagonizing one or more proteases in a subject in need of such treatment.

Another embodiment of the invention is the use of an aspartic protease inhibitor represented by Formula (I), or a pharmaceutically acceptable salt thereof, for the manufacture of a medicament for treating an aspartic protease mediated disorder in a subject.

Another embodiment of the invention is the use of an aspartic protease inhibitor represented by Formula (I), or a pharmaceutically acceptable salt thereof, for therapy, such as treating an aspartic protease mediated disorder in a subject.

Another embodiment of the invention is the use of an aspartic protease inhibitor represented by Formula (I), or a pharmaceutically acceptable salt thereof, for treating a subject having hypertension, congestive heart failure, cardiac hypertrophy, cardiac fibrosis, cardiomyopathy post-infarction, nephropathy, vasculopathy and neuropathy, a disease of the coronary vessels, post-surgical hypertension, restenosis following angioplasty, raised intra-ocular pressure, glaucoma, abnormal vascular growth, hyperaldosteronism, an anxiety state, or a cognitive disorder.

### DETAILED DESCRIPTION OF THE INVENTION

This invention is directed to an aspartic protease inhibitor represented by Formula (I), or a pharmaceutically acceptable salt thereof.

Another embodiment of the invention is an aspartic protease inhibitor, which is a compound represented by Formula (Ia):

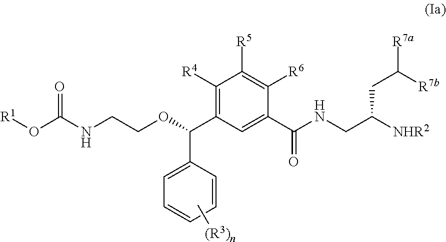

wherein R¹, R², R³, R⁴, R⁵, R⁶, R^{7a}, R^{7b} and n are as defined above, or a pharmaceutically acceptable salt thereof.

In another embodiment, the aspartic protease inhibitor of the present invention is represented by the Formulas (I) or (Ia), wherein: R¹ is C₁-C₃ alkyl; R² is H or C₁-C₃ alkyl; each R³ is independently selected from F, Cl, cyano, nitro, C₁-C₃ alkyl, C₁-C₃ haloalkyl, C₁-C₃ alkoxy, C₁-C₃ haloalkoxy, and C₁-C₃ alkylsulfonyl-; n is 0, 1, or 2; R⁴, R⁵ and R⁶ are selected from H, F, Cl and C₁-C₃ alkyl, wherein one of R⁴, R⁵ or R⁶ is H, F, Cl or C₁-C₃ alkyl and the other two of R⁴, R⁵ and R⁶ are H; and R^{7a} and R^{7b} are each independently C₁-C₃ alkyl, or R^{7a} and R^{7b} taken together with the carbon atom to which they are attached form a 5-6 membered carbocyclic or heterocylic ring, where the heterocyclic ring contains one oxygen atom; or a pharmaceutically acceptable salt thereof.

In another embodiment, the aspartic protease inhibitor of the present invention is represented by Formulas (I) or (Ia), wherein: R¹ is C₁-C₃ alkyl; R² is C₁-C₃ alkyl; each R³ is independently selected from F, Cl and C₁-C₃ alkyl; n is 0, 1 or 2; R⁴, R⁵ and R⁶ are each H or one of R⁴, R⁵ or R⁶ is F, Cl or methyl; and R^{7a} and R^{7b} are each independently C₁-C₃ alkyl, or R^{7a} and R^{7b} taken together with the carbon atom to which they are attached form a cyclohexyl or a tetrahydropyranyl ring; or a pharmaceutically acceptable salt thereof.

Another embodiment of the invention is an aspartic protease inhibitor, which is a compound represented by Formula (II):

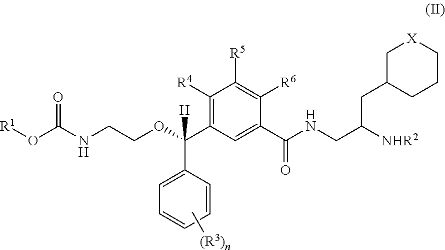

wherein:
R¹ is C₁-C₃ alkyl, C₃-C₆ cycloalkyl or C₃-C₆ cycloalkyl-C₁-C₃ alkyl-;
R² is H or C₁-C₃ alkyl;
each R³ is independently selected from F, Cl, Br, cyano, nitro, C₁-C₃ alkyl, C₁-C₃ haloalkyl, C₁-C₃ alkoxy, C₁-C₃ haloalkoxy, and C₁-C₃ alkylsulfonyl-;
n is 0, 1, 2, or 3;
R⁴, R⁵ and R⁶ are selected from H, halo and C₁-C₃ alkyl, wherein one of R⁴, R⁵ or R⁶ is H, halo or C₁-C₃ alkyl and the other two of R⁴, R⁵ and R⁶ are H;
X is CH₂ or O;
   or a pharmaceutically acceptable salt thereof.

In another embodiment, the aspartic protease inhibitor of the present invention is represented by the Formula (II), wherein: R¹ is C₁-C₃ alkyl; R² is H or C₁-C₃ alkyl; each R³ is independently selected from F, Cl, cyano, nitro, C₁-C₃ alkyl, C₁-C₃ haloalkyl, C₁-C₃ alkoxy, C₁-C₃ haloalkoxy, and C₁-C₃ alkylsulfonyl- and n is 0, 1, or 2 or R³ is selected from F, Cl, cyano, nitro, C₁-C₃ alkyl, C₁-C₃ haloalkyl, C₁-C₃ alkoxy, C₁-C₃ haloalkoxy, and C₁-C₃ alkylsulfonyl- and n is 0 or 1; R⁴, R⁵ and R⁶ are selected from H, F, Cl and C₁-C₃ alkyl, wherein one of R⁴, R⁵ or R⁶ is H, F, C₁ or C₁-C₃ alkyl and the other two of R⁴, R⁵ and R⁶ are H; and X is CH₂ or O; or a pharmaceutically acceptable salt thereof.

In another embodiment, the aspartic protease inhibitor of the present invention is represented by the Formula (II), wherein: R¹ is C₁-C₃ alkyl; R² is C₁-C₃ alkyl; each R³ is independently selected from F, Cl and C₁-C₃ alkyl and n is 0, 1 or 2 or R³ is selected from F, Cl and C₁-C₃ alkyl and n is 0 or 1; R⁴, R⁵ and R⁶ are each H or one of R⁴, R⁵ or R⁶ is F, Cl or methyl; and X is O; or a pharmaceutically acceptable salt thereof.

Another embodiment of the invention is an aspartic protease inhibitor, which is a compound represented by Formula (IIa):

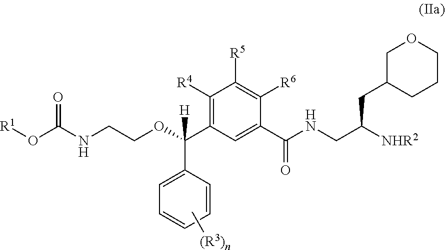

wherein:
R¹ is C₁-C₃ alkyl, C₃-C₆ cycloalkyl or C₃-C₆ cycloalkyl-C₁-C₃ alkyl-;
R² is H or C₁-C₃ alkyl;
each R³ is independently selected from F, Cl, Br, cyano, nitro, C₁-C₃ alkyl, C₁-C₃ haloalkyl, C₁-C₃ alkoxy, C₁-C₃ haloalkoxy, and C₁-C₃ alkylsulfonyl-;
n is 0, 1, 2, or 3; and
R⁴, R⁵ and R⁶ are selected from H, halo and C₁-C₃ alkyl, wherein one of R⁴, R⁵ or R⁶ is H, halo or C₁-C₃ alkyl and the other two of R⁴, R⁵ and R⁶ are H;
   or a pharmaceutically acceptable salt thereof.

In another embodiment, the aspartic protease inhibitor of the present invention is represented by the Formula (IIa), wherein: R¹ is C₁-C₃ alkyl; R² is H or C₁-C₃ alkyl; each R³ is independently selected from F, Cl, cyano, nitro, C₁-C₃ alkyl, C₁-C₃ haloalkyl, C₁-C₃ alkoxy, C₁-C₃ haloalkoxy, and C₁-C₃ alkylsulfonyl- and n is 0, 1, or 2 or R³ is selected from F, Cl, cyano, nitro, C₁-C₃ alkyl, C₁-C₃ haloalkyl, C₁-C₃ alkoxy, C₁-C₃ haloalkoxy, and C₁-C₃ alkylsulfonyl- and n is 0 or 1; and R⁴, R⁵ and R⁶ are selected from H, F, Cl and C₁-C₃ alkyl, wherein one of R⁴, R⁵ or R⁶ is H, F, C₁ or C₁-C₃ alkyl and the other two of R⁴, R⁵ and R⁶ are H; or a pharmaceutically acceptable salt thereof.

In another embodiment, the aspartic protease inhibitor of the present invention is represented by the Formula (IIa), wherein: R¹ is C₁-C₃ alkyl; R² is C₁-C₃ alkyl; each R³ is independently selected from F, Cl and C₁-C₃ alkyl and n is 0, 1 or 2 or R³ is selected from F, Cl and C₁-C₃ alkyl and n is 0 or 1; and R⁴, R⁵ and R⁶ are each H or one of R⁴, R⁵ or R⁶ is F, Cl or methyl; or a pharmaceutically acceptable salt thereof.

Another embodiment of the invention is an aspartic protease inhibitor, which is a compound represented by Formula (IIb):

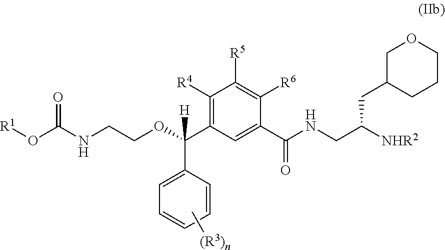

wherein R¹, R², R³, n, R⁴, R⁵ and R⁶ are as defined for Formula (IIa), above, or a pharmaceutically acceptable salt thereof.

In another specific embodiment, the aspartic protease inhibitor is represented by Formulas (I), (Ia), (II), (IIa) or (IIb), wherein R¹ is methyl and R² is methyl and n, R³, R⁴, R⁵, R⁶, and R^{7a} and R^{7b} of Formulas (I) and (Ia) or X of Formula (II) are as defined above, or a pharmaceutically acceptable salt thereof. In another specific embodiment, the aspartic protease inhibitor is represented by Formulas (I), (Ia), (II), (IIa) or (IIb), wherein R¹ is methyl; R² is methyl; each R³ is independently selected from F, Cl and methyl and n is 0, 1 or 2 or R³ is F, Cl or methyl and n is 0 or 1; one of R⁴, R⁵ or R⁶ is F, Cl or methyl or R⁴, R⁵ and R⁶ are each H, and R^{7a} and R^{7b} of Formulas (I) and (Ia) or X of Formula (II), (IIa) or (IIb) are as defined above, or a pharmaceutically acceptable salt thereof.

In another embodiment of the compounds of Formula (I), (Ia), (II), (IIa) or (IIb), R¹ is C₁-C₃ alkyl.

In another embodiment of the compounds of Formula (I), (Ia), (II), (IIa) or (IIb), R² is H or C₁-C₃ alkyl. In a further embodiment of the compounds of Formula (I), (Ia), (II), (IIa) or (IIb), R² is C₁-C₃ alkyl.

In another embodiment of the compounds of Formula (I), (Ia), (II), (IIa) or (IIb), R⁴, R⁵ and R⁶ are selected from H, F, Cl and C₁-C₃ alkyl, wherein one of R⁴, R⁵ or R⁶ is H, F, C₁ or C₁-C₃ alkyl and the other two of R⁴, R⁵ and R⁶ are H.

In another embodiment of the compounds of Formula (I), (Ia), (II), (IIa) or (III)), each R³ is independently selected from F, Cl and C₁-C₃ alkyl.

In another embodiment of the compounds of Formula (I), (Ia), (II), (IIa) or (IIb), n is 0, 1 or 2.

In another embodiment of the compounds of Formula (I), (Ia), (II), (IIa) or (IIb), R¹ is methyl.

In another embodiment of the compounds of Formula (I), (Ia), (II), (IIa) or (IIb), R² is H or methyl. In a further embodiment of the compounds of Formula (I), (Ia), (II), (IIa) or (IIb), R² is methyl.

In another embodiment of the compounds of Formula (I), (Ia), (II), (IIa) or (IIb), each R³ is independently selected from F, Cl, and methyl.

In another embodiment of the compounds of Formula (I), (Ia), (II), (IIa) or (IIb), n is 0.

In another embodiment of the compounds of Formula (I), (Ia), (II), (IIa) or (IIb), n is 1.

In another embodiment of the compounds of Formula (I), (Ia), (II), (IIa) or (IIb), n is 2.

In another embodiment of the compounds of Formula (I), (Ia), (II), (IIa) or (IIb), R³ is F and n is 1.

In another embodiment of the compounds of Formula (I), (Ia), (II), (IIa) or (IIb), R³ is Cl and n is 1.

In another embodiment of the compounds of Formula (I), (Ia), (II), (IIa) or (IIb), n is 2, one R³ is Cl and the other R³ is methyl.

In another embodiment of the compounds of Formula (I), (Ia), (II), (IIa) or (IIb), R⁴, R⁵ and R⁶ are each H.

In another embodiment of the compounds of Formula (I), (Ia), (II), (IIa) or (IIb), one of R⁴, R⁵ or R⁶ is F, Cl or methyl.

The present invention contemplates and includes any and all combinations of the embodiments of R¹, R², R³, R⁴, R⁵, R⁶, and n, as defined herein.

In another specific embodiment, the aspartic protease inhibitor of the present invention is one of the compounds in Table 1, or an enantiomer or diastereomer thereof (where the enantiomer or diastereomer is of any non-specified chiral center; the specified chiral center remaining as depicted). Also included are pharmaceutically acceptable salts and solvates (e.g., hydrates) of the compounds in Table 1, or an enantiomer or diastereomer thereof.

TABLE 1

| Cpd | | |
|---|---|---|
| No. | Structure | Name |
| I-1 | 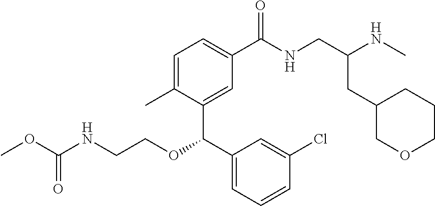 | methyl [2-({(3-chlorophenyl)[2- methyl-5-({[2-(methylamino)-3- (tetrahydro-2H-pyran-3- yl)propyl]amino}carbonyl)phenyl] methyl}oxy)ethyl]carbamate |
| I-2 | 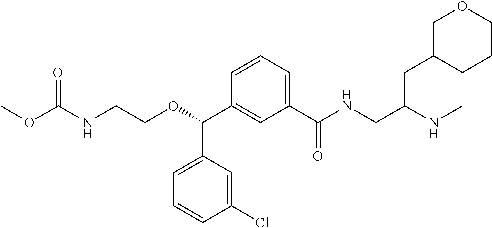 | methyl [2-({(3-chlorophenyl)[3- ({[2-(methylamino)-3-(tetrahydro- 2H-pyran-3-yl)propyl]amino} carbonyl)phenyl]methyl}oxy) ethyl]carbamate |
| I-3 | 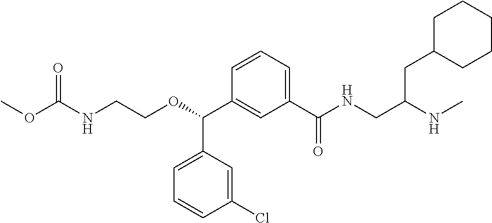 | methyl [2-({(3-chlorophenyl)[3- ({[3-cyclohexyl-2- (methylamino)propyl]amino} carbonyl)phenyl]methyl}oxy) ethyl]carbamate |
| I-4 | 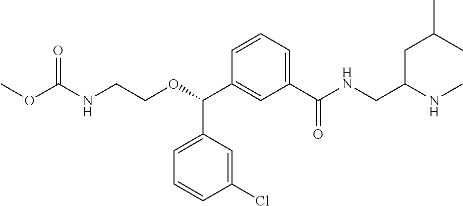 | methyl [2-({(3-chlorophenyl)[3- ({[4-methyl-2-(methylamino) pentyl]amino}carbonyl)phenyl] methyl}oxy) ethyl]carbamate |
| I-5 | 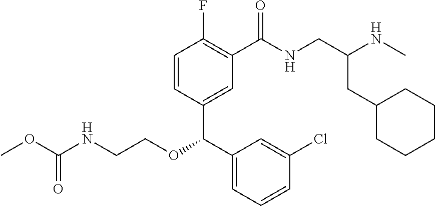 | methyl [2-({(3-chlorophenyl)[3- ({[3-cyclohexyl-2- (methylamino)propyl] amino}carbonyl)-4-fluorophenyl] methyl}oxy)ethyl]carbamate |
| I-6 | 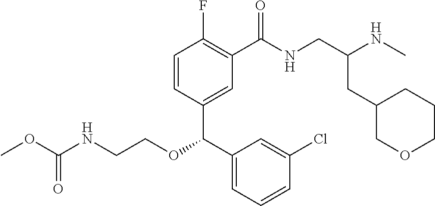 | methyl [2-({(3-chlorophenyl)[4- fluoro-3-({[2-(methylamino)-3- (tetrahydro-2H-pyran-3-yl)propyl] amino}carbonyl)phenyl]methyl} oxy) ethyl]carbamate |
| I-7 | 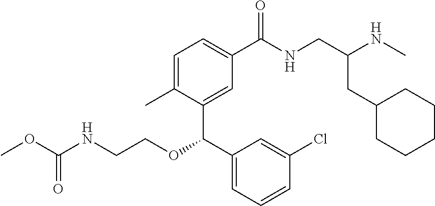 | methyl [2-({(3-chlorophenyl)[5- ({[3-cyclohexyl-2- (methylamino)propyl]amino} carbonyl)-2-methylphenyl] methyl}oxy)ethyl]carbamate |
| I-8 | 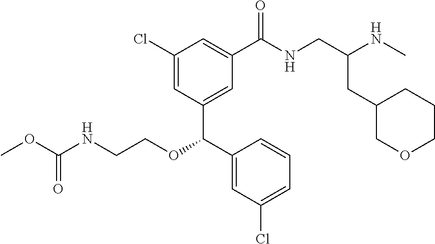 | methyl (2-{[[3-chloro-5-({[2- (methylamino)-3-(tetrahydro-2H- pyran-3-yl)propyl]amino} carbonyl)phenyl](3-chlorophenyl) methyl]oxy}ethyl)carbamate |
| I-9 | 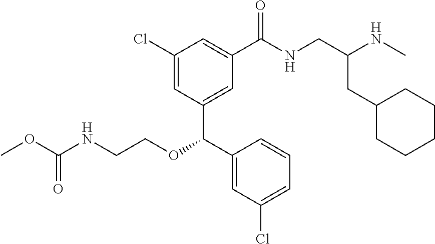 | methyl (2-{[[3-chloro-5-({[3- cyclohexyl-2-(methylamino) propyl]amino}carbonyl)phenyl] (3-chlorophenyl)methyl]oxy} ethyl)carbamate |
| I-10 | 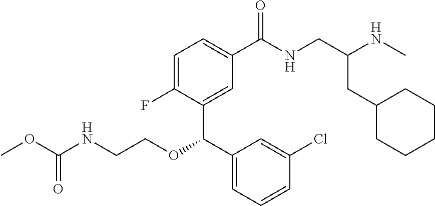 | methyl [2-({(3-chlorophenyl)[5- ({[3-cyclohexyl-2- (methylamino)propyl] amino}carbonyl)-2-fluorophenyl] methyl}oxy)ethyl]carbamate |
| I-11 | 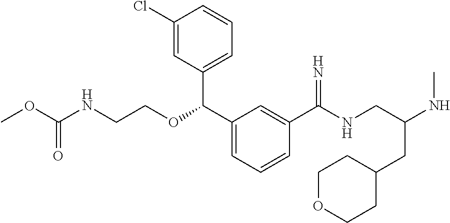 | methyl [2-({(3-chlorophenyl)[3- ({[2-(methylamino)-3-(tetrahydro- 2H-pyran-4-yl)propyl]amino} carbonyl)phenyl]methyl}oxy) ethyl]carbamate |
| I-12 | 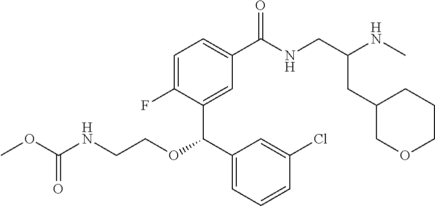 | methyl [2-({(3-chlorophenyl)[2- fluoro-5-({[2-(methylamino)-3- (tetrahydro-2H-pyran-3-yl)propyl] amino}carbonyl)phenyl]methyl} oxy) ethyl]carbamate |
| I-13 | 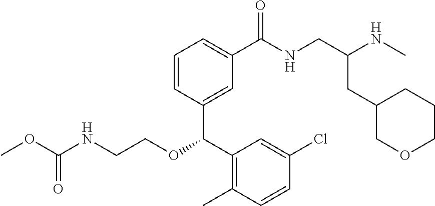 | methyl [2-({(5-chloro-2- methylphenyl)[3-({[2- (methylamino)-3-(tetrahydro-2H- pyran-3-yl)propyl]amino} carbonyl)phenyl]methyl}oxy) ethyl]carbamate |

Selected aspartic protease inhibitors of Formulas (I), (Ia), (II), (IIa) or (IIb), include the compounds of Table 2, and the pharmaceutically acceptable salts and solvates (e.g., hydrates) thereof.

TABLE 2

| Cpd | | |
|---|---|---|
| No. | Structure | Name |
| I-1a* | 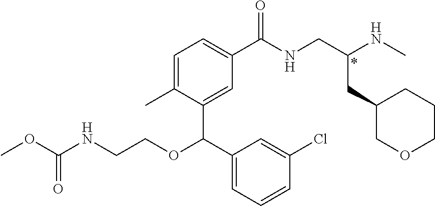 | methyl {2-[((3-chlorophenyl){2- methyl-5-[({(2S)-2-(methylamino)- 3-[(3R)-tetrahydro-2H-pyran-3-yl] propyl}amino)carbonyl]phenyl} methyl)oxy]ethyl}carbamate |
| I-1b | 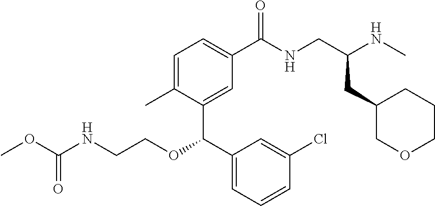 | methyl {2-[((S)-(3-chlorophenyl){2- methyl-5-[({(2S)-2-(methylamino)- 3-[(3R)-tetrahydro-2H-pyran-3- yl]propyl}amino)carbonyl]phenyl} methyl)oxy]ethyl}carbamate |
| I-2a* | 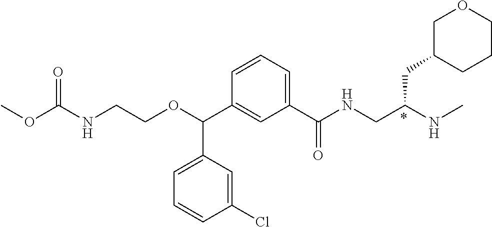 | methyl {2-[((3-chlorophenyl){3- [({(2S)-2-(methylamino)-3-[(3R)- tetrahydro-2H-pyran-3-yl] propyl}amino)carbonyl]phenyl} methyl)oxy]ethyl}carbamate |
| I-2b | 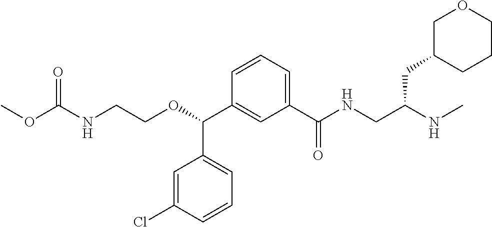 | methyl {2-[((R)-(3-chlorophenyl){3- [({(2S)-2-(methylamino)-3-[(3R)- tetrahydro-2H-pyran-3-yl]propyl} amino)carbonyl]phenyl} methyl)oxy]ethyl}carbamate |
| I-2c | 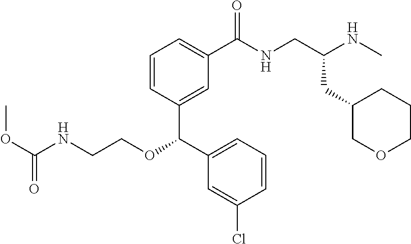 | methyl {2-[((R)-(3-chlorophenyl){3- [({(2R)-2-(methylamino)-3-[(3S)- tetrahydro-2H-pyran-3-yl] propyl}amino)carbonyl]phenyl} methyl)oxy]ethyl}carbamate |
| I-2d | 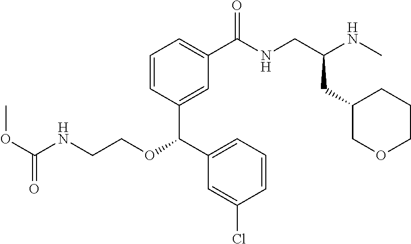 | methyl {2-[((R)-(3-chlorophenyl){3- [({(2S)-2-(methylamino)-3-[(3S)- tetrahydro-2H-pyran-3-yl] propyl}amino)carbonyl]phenyl} methyl)oxy]ethyl}carbamate |
| I-2e | 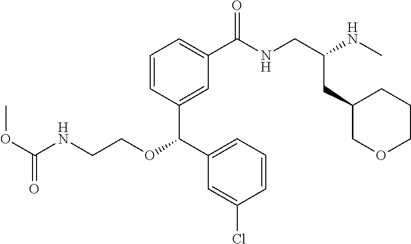 | methyl {2-[((R)-(3-chlorophenyl){3- [({(2R)-2-(methylamino)-3-[(3R)- tetrahydro-2H-pyran-3-yl] propyl}amino)carbonyl]phenyl} methyl)oxy]ethyl}carbamate |
| I-3a | 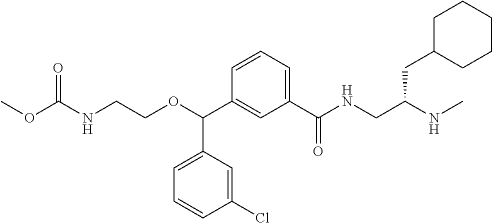 | methyl [2-({(3-chlorophenyl)[3- ({[(2S)-3-cyclohexyl-2- (methylamino)propyl]amino} carbonyl)phenyl]methyl}oxy)ethyl] carbamate |
| I-4a | 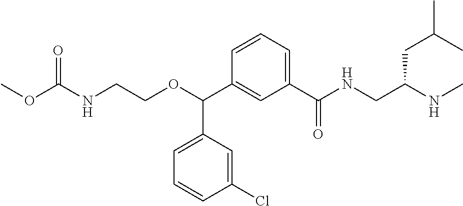 | methyl [2-({(3-chlorophenyl)[3- ({[(2S)-4-methyl-2-(methylamino) pentyl]amino}carbonyl)phenyl] methyl}oxy)ethyl]carbamate |
| I-5a | 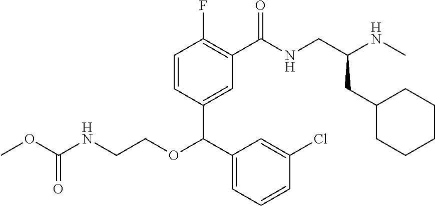 | methyl [2-({(3-chlorophenyl)[3- ({[(2S)-3-cyclohexyl-2- (methylamino)propyl]amino} carbonyl)-4-fluorophenyl]methyl} oxy)ethyl] carbamate |
| I-6a* | 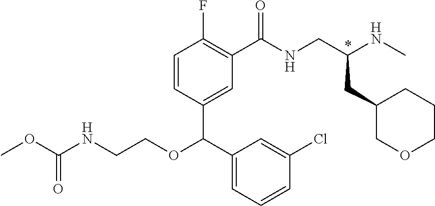 | methyl {2-[((3-chlorophenyl){4- fluoro-3-[({(2S)-2-(methylamino)-3- [(3R)-tetrahydro-2H-pyran-3-yl] propyl}amino)carbonyl]phenyl} methyl)oxy]ethyl}carbamate |
| I-7a | 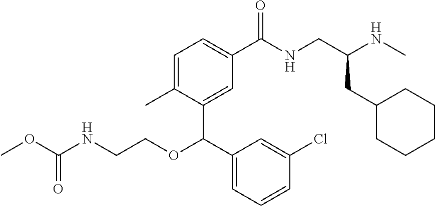 | methyl [2-({(3-chlorophenyl)[5- ({[(2S)-3-cyclohexyl-2- (methylamino)propyl]amino} carbonyl)-2-methylphenyl]methyl} oxy)ethyl] carbamate |
| I-8a | 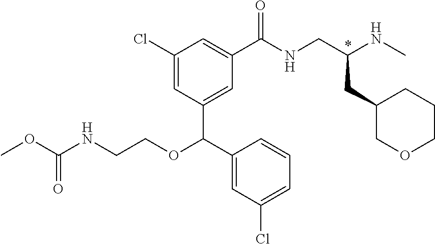 | methyl (2-{[{3-chloro-5-[({(2S)-2- (methylamino)-3-[(3R)-tetrahydro- 2H-pyran-3-yl]propyl}amino) carbonyl] phenyl}(3-chlorophenyl) methyl]oxy}ethyl)carbamate |
| I-9a | 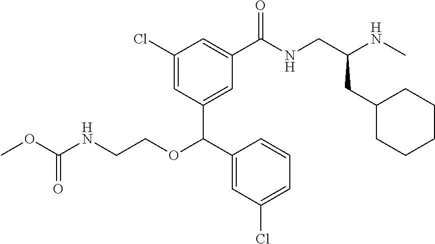 | methyl (2-{[[3-chloro-5-({[(2S)-3- cyclohexyl-2-(methylamino)propyl] amino}carbonyl)phenyl](3- chlorophenyl)methyl]oxy}ethyl) carbamate |
| I-10a | 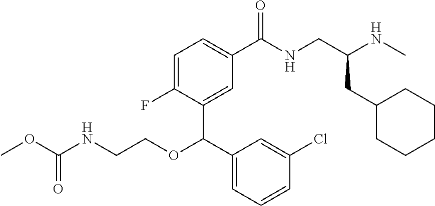 | methyl [2-({(3-chlorophenyl)[5- ({[(2S)-3-cyclohexyl-2- (methylamino)propyl]amino} carbonyl)-2-fluorophenyl] methyl}oxy)ethyl] carbamate |
| I-11a | 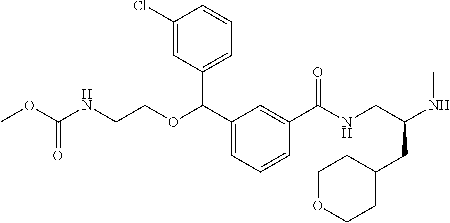 | methyl [2-({(3-chlorophenyl)[3- ({[(2S)-2-(methylamino)-3- (tetrahydro-2H-pyran-4- yl)propyl]amino}carbonyl)phenyl] methyl}oxy)ethyl]carbamate |
| I-12a* | 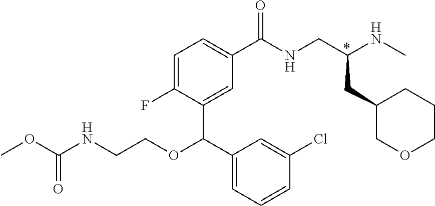 | methyl {2-[((3-chlorophenyl){2- fluoro-5-[({(2S)-2-(methylamino)-3- [(3R)-tetrahydro-2H-pyran-3- yl]propyl}amino)carbonyl]phenyl} methyl)oxy]ethyl}carbamate |
| I-13a | 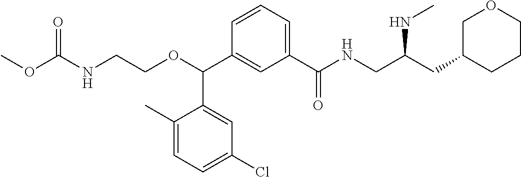 | methyl {2-[((5-chloro-2- methylphenyl){3-[({(2S)-2- (methylamino)-3-[(3R)-tetrahyrdo- 2H-pyran-3-yl]propyl}amino) carbonyl] phenyl}methyl)oxy] ethyl}carbamate |
| I-13b | 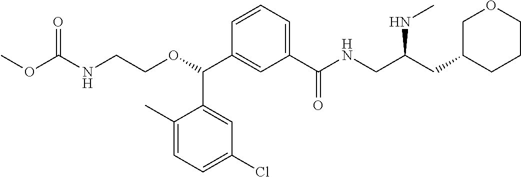 | methyl {2-[((R)-(5-chloro-2- methylphenyl){3-[({(2S)-2- (methylamino)-3-[(3R)-tetrahydro- 2H-pyran-3-yl]propyl}amino) carbonyl]phenyl}methyl)oxy] ethyl}carbamate |

*These compounds were prepared, isolated and evaluated as a 4:1 mixture of stereoisomers at the designated center.

Intermediates useful for synthesizing the aspartic protease inhibitors disclosed herein are represented by Formulas (IV), (IVa), (IVb), (IVc) or (IVd) and salts thereof (preferably pharmaceutically acceptable salts):

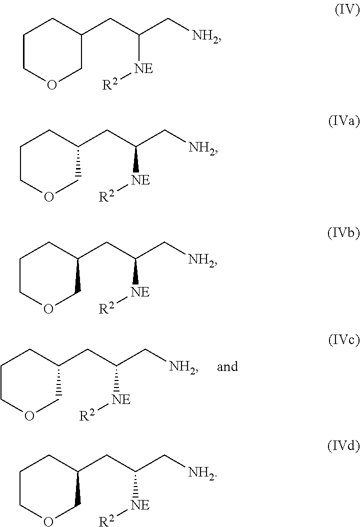

In Formulas (IV), (IVa), (IVb), (IVc), and (IVd), E is H or an amine protecting group. Amine protecting groups include carbamate, amide, and sulfonamide protecting groups known in the art (T. W. Greene and P. G. M. Wuts “Protective Groups in Organic Synthesis” John Wiley & Sons, Inc., New York 1999) and the entire teaching of which is herein incorporated by reference. Specific amine protecting groups include tert-butoxycarbonyl (Boc), benzyloxycarbonyl (Cbz) and 1-[2-(trimethylsilyl)ethoxycarbonyl] (Teoc). More specifically, the amine protecting group is tert-butoxycarbonyl (Boc). Values and specific values for R² are as described for Formula (I).

Specific intermediates useful for the preparation of the aspartic protease inhibitors of this invention include each of the following compounds, or their enantiomers or diastereomers. Pharmaceutically acceptable salts of the following are also included:

| Compound No. | Compound Name |
|---|---|
| IV-1 | tert-butyl (S)-1-amino-3-((R)-tetrahydro-2H-pyran- |
| | 3-yl) propan-2-ylcarbamate |
| IV-2 | tert-butyl (S)-1-amino-3-((R)-tetrahydro-2H-pyran- |
| | 3-yl) propan-2-yl(methyl)carbamate |
| IV-1 | tert-butyl-1-amino-3-(tetrahydro-2H-pyran-3-yl) |
| | propan-2-ylcarbamate |
| IV-2 | tert-butyl-1-amino-3-(tetrahydro-2H-pyran-3-yl) |
| | propan-2-yl(methyl)carbamate |

When any variable (e.g., R³) occurs more than once in a compound, its definition on each occurrence is independent of any other occurrence. For example, R³, for each occurrence, is independently selected from the group consisting of F, Cl, Br, cyano, nitro, alkyl, haloalkyl, alkoxy, haloalkoxy, and alkanesulfonyl.

When the “aspartic protease inhibitor” of the present invention is named or depicted by structure, it also includes pharmaceutically acceptable salts thereof.

“Alkyl”, alone or part of another moiety (such as cycloalkylalkyl, alkoxy, haloalkoxy, haloalkyl or alkoxy), means a saturated aliphatic branched or straight-chain mono- or divalent hydrocarbon radical. Alkyls commonly have from one to six carbon atoms, typically from one to three carbon atoms. Thus, “(C₁-C₃)alkyl” means a radical having from 1-3 carbon atoms in a linear or branched arrangement. “(C₁-C₃)alkyl” includes methyl, ethyl, propyl and isopropyl.

“Cycloalkyl”, alone or as part of another moiety (such as cycloalkylalkyl) means a saturated aliphatic cyclic mono-valent hydrocarbon radical. Typically, cycloalkyls have from three to ten carbon atoms and are mono, bi or tricyclic. Tricyclic cycloalkyls can be fused or bridged. Typically, cycloalkyls are C₃-C₈ monocyclic and are more commonly cyclopropyl.

“Cycloalkylalkyl” means an alkyl radical substituted with a cycloalkyl group.

“Haloalkyl” includes mono, poly, and perhaloalkyl groups where the halogens are independently selected from fluorine, chlorine, and bromine.

“Alkoxy” means an alkyl radical attached through an oxygen linking atom. “(C₁-C₃)-alkoxy” includes the methoxy, ethoxy, and propoxy.

“Haloalkoxy” is a haloalkyl group which is attached to another moiety via an oxygen linker.

“Alkanesulfonyl” is an alkyl radical attached through a

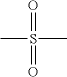

linking group. “(C₁-C₃)alkanesulfonyl” includes methanesulfonyl, ethanesulfonyl and propanesulfonyl.

Certain of the disclosed aspartic protease inhibitors may exist in various tautomeric forms. The invention encompasses all such forms, including those forms not depicted structurally.

Certain of the disclosed aspartic protease inhibitors may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. When a chiral center is not defined as R or S and the configuration at the chiral center is not defined by other means, either configuration can be present or a mixture of both configurations is present.

“Racemate” or “racemic mixture” means a compound of equimolar quantities of two enantiomers, wherein such mixtures exhibit no optical activity; i.e., they do not rotate the plane of polarized light.

“R” and “S” indicate configurations relative to the core molecule.

[symbol] or [symbol] represents [symbol] or [symbol] wherein the depicted enantiomer (e.g., [symbol] or [symbol] ) is at least 60%, 70%, 80%, 90%, 99% or 99.9% optically pure.

The disclosed aspartic protease inhibitors, as well as many of the intermediates used to prepare the inhibitors, may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed aspartic protease inhibitor or an intermediate is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% enantiomerically pure.

When a disclosed aspartic protease inhibitor or an intermediate is named or depicted by structure without indicating the stereochemistry, and the inhibitor or intermediate has at least one chiral center, it is to be understood that the name or structure encompasses one enantiomer of the inhibitor or intermediate free from the corresponding enantiomer/optical isomer, a racemic mixture of the inhibitor or intermediate and mixtures enriched in one enantiomer relative to its corresponding enantiomer/optical isomer.

When a disclosed aspartic protease inhibitor or intermediate is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

When a disclosed aspartic protease inhibitor or an intermediate is named or depicted by structure with indication of the stereochemistry, and the inhibitor or intermediate has at least one chiral center, it is to be understood that the name or structure encompasses one enantiomer of the inhibitor or intermediate free from the corresponding enantiomer/optical isomer as well as mixtures enriched in the one depicted enantiomer relative to its corresponding enantiomer/optical isomer.

When a disclosed aspartic protease inhibitor or intermediate is named or depicted by structure with indication the stereochemistry and has at least two chiral centers, it is to be understood that the name or structure encompasses a diastereomer free of other diastereomers as well as mixtures of diastereomers in which the depicted diastereomer is enriched relative to the other diastereomer(s) and mixtures of diastereomeric pairs in which the depicted diastereomeric pair is enriched relative to the other diastereomeric pair(s).

Pharmaceutically acceptable salts of the compounds of the aspartic protease inhibitors are included in the present invention. For example, an acid salt of an aspartic protease inhibitor containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

Salts of the compounds of aspartic protease inhibitors containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

In accordance with the present invention, non-pharmaceutically acceptable salts of the compounds of the aspartic protease inhibitors and their synthetic intermediates are also included. These salts (for example, TFA salt) may be used, for example, for purification and isolation of the compounds of the aspartic protease inhibitors and their synthetic intermediates.

When a disclosed aspartic protease inhibitor is named or depicted by structure, it is to be understood that solvates (e.g., hydrates) of the aspartic protease inhibitor are also included. “Solvates” refer to crystalline forms wherein solvent molecules are incorporated into the crystal lattice during crystallization. Solvates may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates.” Hydrates include stoichiometric hydrates (a monohydrate) as well as compositions containing variable amounts of water (e.g., a hemi-hydrate, a dihydrate, etc).

When a disclosed aspartic protease inhibitor is named or depicted by structure, it is to be understood that the compound or its pharmaceutically acceptable salt, including solvates thereof, may exist in crystalline forms, non-crystalline forms or a mixture thereof. The aspartic protease inhibitor or solvates may also exhibit polymorphism (i.e. the capacity to occur in different crystalline forms). These different crystalline forms are typically known as “polymorphs.” It is to be understood that when named or depicted by structure, the disclosed aspartic protease inhibitors and their solvates (e.g., hydrates) also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999, and the entire teaching of which is herein incorporated by reference. Protecting groups may be added and removed using methods well known in the art.

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; arial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels, proteinuria, albumenuria, post-surgical hypertension, metabolic syndrome, obesity, restenosis following angioplasty, eye diseases and associated abnormalities including raised intra-ocular pressure, glaucoma, retinopathy, abnormal vascular growth and remodelling, angiogenesis-related disorders, such as neovascular age related macular degeneration; hyperaldosteronism, anxiety states, and cognitive disorders (Fisher N. D.; Hollenberg N. K. Expert Opin. Investig. Drugs. 2001, 10, 417-26).

Elevated levels of βamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a disclosed aspartic protease inhibitor, comprise a prodrug or pharmaceutically active metabolite of such a compound or salt and one or more pharmaceutically acceptable carriers or diluent therefor.

The invention includes a therapeutic method for treating or ameliorating an aspartic protease mediated disorder in a subject in need thereof comprising administering to a subject in need thereof an effective amount of an aspartic protease inhibitor disclosed herein.

Administration methods include administering an effective amount of a compound or composition of the invention at different times during the course of therapy or concurrently in a combination form. The methods of the invention include all known therapeutic treatment regimens.

“Effective amount” means that amount of drug substance (i.e. aspartic protease inhibitors of the present invention) that elicits the desired biological response in a subject. Such response includes alleviation of the symptoms of the disease or disorder being treated. The effective amount of a disclosed aspartic protease inhibitor in such a therapeutic method is from about 0.01 mg/kg/day to about 10 mg/kg/day, preferably from about 0.5 mg/kg/day to 5 mg/kg/day.

The invention includes the use of a disclosed aspartic protease inhibitor for the preparation of a composition for treating or ameliorating an aspartic protease mediated chronic disorder or disease or infection in a subject in need thereof, wherein the composition comprises a mixture of one or more of the disclosed aspartic protease inhibitors and an optional pharmaceutically acceptable carrier.

“Pharmaceutically acceptable carrier” means compounds and compositions that are of sufficient purity and quality for use in the formulation of a composition of the invention that, when appropriately administered to an animal or human, do not produce an adverse reaction, and that are used as a vehicle for a drug substance (i.e. aspartic protease inhibitors of the present invention).

“Pharmaceutically acceptable diluent” means compounds and compositions that are of sufficient purity and quality for use in the formulation of a composition of the invention that, when appropriately administered to an animal or human, do not produce an adverse reaction, and that are used as a diluting agent for a drug substance (i.e. aspartic protease inhibitors of the present invention).

“Aspartic protease mediated disorder or disease” includes disorders or diseases associated with the elevated expression or overexpression of aspartic proteases and conditions that accompany such diseases.

An embodiment of the invention includes administering an aspartic protease inhibitor disclosed herein in a combination therapy (see U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8, the aforementioned article and patents are hereby incorporated by reference) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

Centrally acting antiphypertensives include clonidine, guanabenz, guanfacine and methyldopa.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

An embodiment of the invention includes administering an aspartic protease inhibitor disclosed herein or composition thereof in a combination therapy with one or more additional agents for the treatment of AIDS reverse transcriptase inhibitors, non-nucleoside reverse transcriptase inhibitors, other HIV protease inhibitors, HIV integrase inhibitors, entry inhibitors (including attachment, co-receptor and fusion inhibitors), antisense drugs, and immune stimulators.

Preferred reverse transcriptase inhibitors are zidovudine, didanosine, zalcitabine, stavudine, lamivudine, abacavir, tenofovir, and emtricitabine.

Preferred non-nucleoside reverse transcriptase inhibitors are nevirapine, delaviridine, and efavirenz.

Preferred HIV protease inhibitors are saquinavir, ritonavir, indinavir, nelfinavir, amprenavir, lopinavir, atazanavir, and fosamprenavir.

Preferred HIV integrase inhibitors are L-870,810 and S-1360.

Entry inhibitors include compounds that bind to the CD4 receptor, the CCR5 receptor or the CXCR4 receptor. Specific examples of entry inhibitors include enfuvirtide (a peptidomimetic of the HR2 domain in gp41) and sifurvitide.

A preferred attachment and fusion inhibitor is enfuvirtide.

An embodiment of the invention includes administering an aspartic protease inhibitor disclosed herein or composition thereof in a combination therapy with one or more additional agents for the treatment of Alzheimer's disease including tacrine, donepezil, rivastigmine, galantamine, and memantine.

An embodiment of the invention includes administering an aspartic protease inhibitor disclosed herein or composition thereof in a combination therapy with one or more additional agents for the treatment of malaria including artemisinin, chloroquine, halofantrine, hydroxychloroquine, mefloquine, primaquine, pyrimethamine, quinine, sulfadoxine.

Combination therapy includes co-administration of an aspartic protease inhibitor disclosed herein and said other agent, sequential administration of the disclosed aspartic protease inhibitor and the other agent, administration of a composition containing the aspartic protease inhibitor and the other agent, or simultaneous administration of separate compositions containing the aspartic protease inhibitor and the other agent.

The invention further includes the process for making the composition comprising mixing one or more of the disclosed aspartic protease inhibitors and an optional pharmaceutically acceptable carrier; and includes those compositions resulting from such a process, which process includes conventional pharmaceutical techniques. For example, an aspartic protease inhibitor disclosed herein may be nanomilled prior to formulation. An aspartic protease inhibitor disclosed herein may also be prepared by grinding, micronizing or other particle size reduction methods known in the art. Such methods include, but are not limited to, those described in U.S. Pat. Nos. 4,826,689, 5,145,684, 5,298,262, 5,302,401, 5,336,507, 5,340,564, 5,346,702, 5,352,459, 5,354,560, 5,384,124, 5,429,824, 5,503,723, 5,510,118, 5,518,187, 5,518,738, 5,534,270, 5,536,508, 5,552,160, 5,560,931, 5,560,932, 5,565,188, 5,569,448, 5,571,536, 5,573,783, 5,580,579, 5,585,108, 5,587,143, 5,591,456, 5,622,938, 5,662,883, 5,665,331, 5,718,919, 5,747,001, PCT applications WO 93/25190, WO 96/24336, and WO 98/35666, each of which is incorporated herein by reference. The pharmaceutical compositions of the invention may be prepared using techniques and methods known to those skilled in the art. Some of the methods commonly used in the art are described in Remington's Pharmaceutical Sciences (Mack Publishing Company), the entire teachings of which are incorporated herein by reference.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The dosage form containing the composition of the invention contains an effective amount of the drug substance (i.e. aspartic protease inhibitors of the present invention) necessary to provide a therapeutic and/or prophylactic effect. The composition may contain from about 5,000 mg to about 0.5 mg (preferably, from about 1,000 mg to about 0.5 mg) of a disclosed aspartic protease inhibitor or salt form thereof and may be constituted into any form suitable for the selected mode of administration. The compositions of the invention may be administered in a form suitable for once-weekly or once-monthly administration. For example, an insoluble salt of the drug substance (i.e. aspartic protease inhibitors of the present invention) may be adapted to provide a depot preparation for intramuscular injection (e.g., a decanoate salt) or to provide a solution for ophthalmic administration. Daily administration or post-periodic dosing may also be employed, wherein the composition may be administered about 1 to about 5 times per day.

For oral administration, the composition is preferably in the form of a tablet or capsule containing, e.g., 1000 to 0.5 milligrams of the drug substance (i.e. aspartic protease inhibitors of the present invention), more specifically 500 mg to 5 mg. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the drug substance (i.e. aspartic protease inhibitors of the present invention) is dispersed evenly throughout the mixture, which may be readily subdivided into dosage units containing equal amounts of a disclosed aspartic protease inhibitor. Preferably, the compositions are prepared by mixing a disclosed aspartic protease inhibitor with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binding agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

The disclosed aspartic protease inhibitors may also be administered via a slow release composition, wherein the composition includes a disclosed aspartic protease inhibitor and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain a drug substance(s) (i.e. aspartic protease inhibitors of the present invention) and which slowly degrade/dissolve in a suitable environment (e.g., aqueous, acidic, basic and the like) to release the drug substance(s). Such particles degrade/dissolve in body fluids to release the drug substance(s) (i.e. aspartic protease inhibitors of the present invention) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a disclosed aspartic protease inhibitor are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the disclosed aspartic protease inhibitor.

The disclosed aspartic protease inhibitors may be incorporated for administration orally or by injection in a liquid form, such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The disclosed aspartic protease inhibitors may be administered parenterally via injection. A parenteral formulation may consist of the drug substance (i.e. aspartic protease inhibitors of the present invention) dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the drug substance (i.e. aspartic protease inhibitors of the present invention) in the liquid carrier whereby the final dosage unit contains from 0.005 to 10% by weight of the drug substance (i.e. aspartic protease inhibitors of the present invention). Other additives include preservatives, isotonizers, solubilizers, stabilizers, and pain-soothing agents. Injectable suspensions may also be prepared, in which case appropriate liquid carriers, suspending agents and the like may be employed.

The disclosed aspartic protease inhibitors may be administered intranasally using a suitable intranasal vehicle.

The disclosed aspartic protease inhibitors may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the disclosed aspartic protease inhibitor, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art.

The following abbreviations have the indicated meanings:

| Abbreviation | Meaning |
|---|---|
| Aq | Aqueous |
| Boc | tert-butoxy carbonyl or t-butoxy carbonyl |
| (Boc)₂O | di-tert-butyl dicarbonate |
| Brine | saturated aqueous NaCl |
| Cbz | Benzyloxycarbonyl |
| CbzCl | Benzyl chloroformate |
| CDI | carbonyl diimidazole |
| CH₂Cl₂ | methylene chloride |
| CH₃CN or MeCN | Acetonitrile |
| Cpd | Compound |
| D | Day |
| DAMP | 4,4′-(2-pyridinylmethylene)diphenol acetate |
| DAST | diethylaminosulfur trifluoride |
| DBU | 1,8-diazabicyclo[5.4.0]undec-7-ene |
| DCC | N,N′-dicyclohexylcarbodiimide |
| DCM | Dichloromethane |
| DCU | N,N′-dicyclohexylurea |
| DIAD | diisopropyl azodicarboxylate |
| DiBAlH | Diisobutylaluminum hydride |
| DIEA | N,N-diisopropylethylamine |
| DMAP | 4-(dimethylamino)pyridine |
| DMF | N,N-dimethylform amide |
| DMPU | 1,3-dimethyl-3,4,5,6-tetrahydro-2(1H)-pyrimidinone |
| 2,4-DNP | 2,4-dinitrophenylhydrazine |
| DPPA | Diphenylphosphoryl azide |
| EDCI•HCl | 1-[3-(dimethylamino)propyl]-3-ethylcarbodiimide |
| | hydrochloride |
| Equiv | Equivalents |
| Et | Ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | Hour |
| HOBt | 1-hydroxybenzotriazole |
| HATU | 2-(7-Aza-1H-benzotriazole-1-yl)-1,1,3,3- |
| | tetramethyluronium hexafluorophosphate |
| HBTU | 2-(1H-Benzotriazole-1-yl)-1,1,3,3- |
| | tetramethyluronium hexafluorophosphate |
| KHMDS | potassium hexamethyldisilazane |
| LiHMDS | lithium hexamethyldisilazane |
| LAB | lithium amidotrihydroborate |
| LAH or LiAlH₄ | lithium aluminum hydride |
| LC-MS | liquid chromatography-mass spectroscopy |
| LHMDS | lithium hexamethyldisilazane |
| Me | Methyl |
| MeCN | Acetonitrile |
| MeOH | Methanol |
| MsCl | methanesulfonyl chloride |
| Min | Minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMM | N-methylmorpholine |
| NMP | N-methylpyrrolidinone |
| Pd₂(dba)₃ | tris(dibenzylideneacetone)dipalladium(0) |
| PE | petroleum ether |
| Ph | Phenyl |
| PTSA | p-toluene sulfonic acid |
| R-CBS | (R)-CBS-oxazaborolidine |
| Quant | quantitative yield |
| Rt | room temperature |
| Satd | Saturated |
| SOCl₂ | thionyl chloride |
| SPE | solid phase extraction |
| TBDPSCl | tert-butyl diphenyl silyl chloride |
| TBME | tert-butyl methyl ether |
| TBS | t-butyldimethylsilyl |
| TBSCl | t-butyldimethylsilyl chloride |
| TEA | triethylamine or Et₃N |
| TEAF | tetraethylammonium fluoride |
| TEMPO | 2,2,6,6-tetramethyl-1-piperidinyloxy free radical |
| Teoc | 1-[2-(trimethylsilyl)ethoxycarbonyl] |
| Teoc-OSu | 1-[2-(trimethylsilyl)ethoxycarbonyloxy]pyrrolidin- |
| | 2,5-dione |
| TFA | trifluoroacetic acid |
| THF | Tetrahydrofuran |
| Tlc | thin layer chromatography |
| TMS | Trimethylsilyl |
| TMSCl | chlorotrimethylsilane or trimethylsilyl chloride |
| t_{R} | retention time |
| TsOH | p-toluenesulfonic acid |
| TsCl | p-toluenesulfonyl chloride |
| Red-Al | sodium bis(2-methoxyethoxy)aluminum dihydride |

### General Synthetic Schemes

The compounds of present invention can be synthesized by coupling a pyran intermediate represented by the following structure:

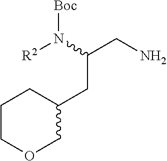

with a benzoic acid intermediate represented by the following structure:

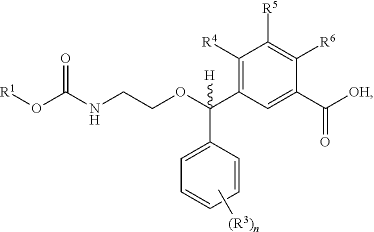

described in the following scheme:

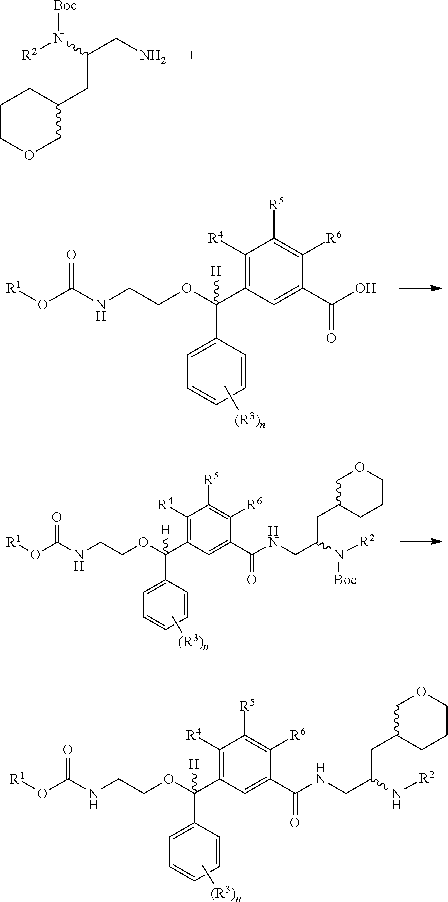

### Preparation of the Pyran Intermediate

The pyran intermediate may be prepared from pyroglutamic ester using the following synthetic scheme:

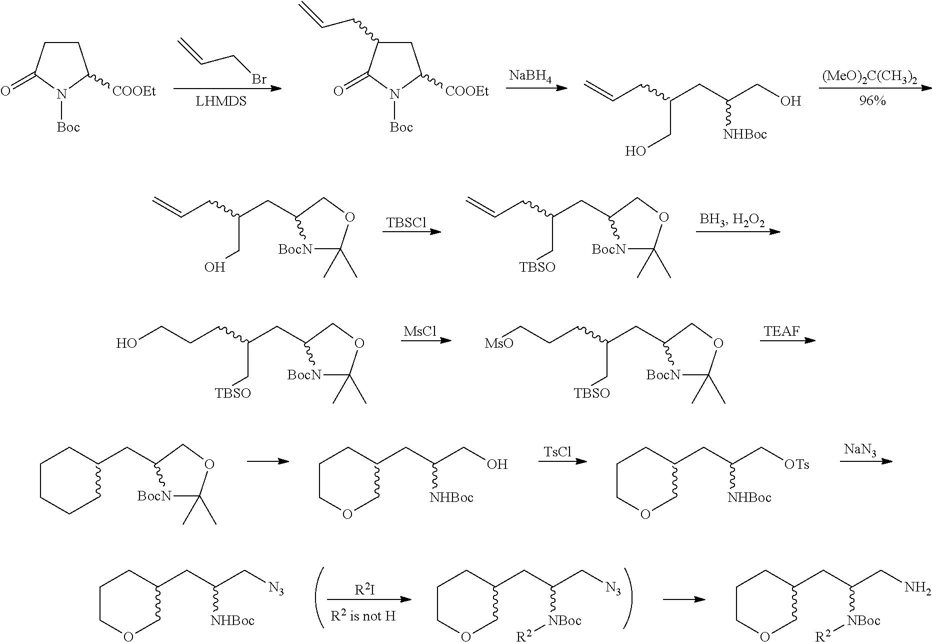

### Preparation of Diastereomerically Pure Pyran Intermediate

The chiral pyran intermediate may be obtained in diastereomerically pure form using the following synthetic scheme:

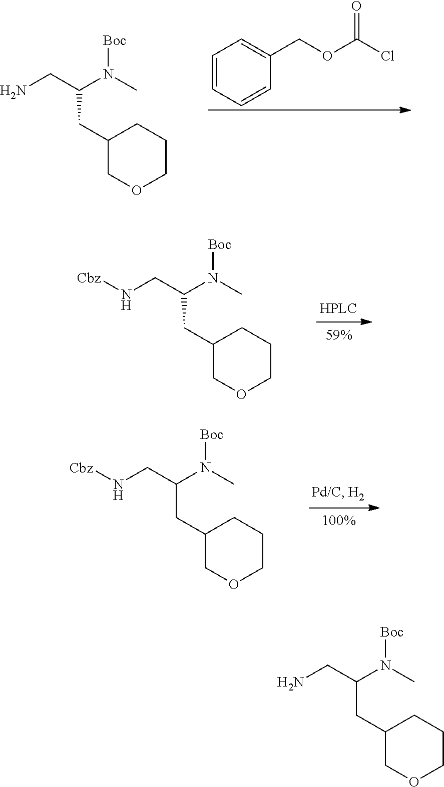

### Preparation of the Benzoic Acid Intermediate

An intermediate that is used in each of the methods for preparing the benzoic acid intermediate is a carbamate-protected amino-ethanol, which can be prepared using the following synthetic scheme.

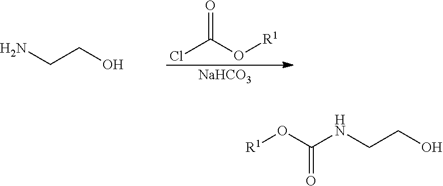

The benzoic acid intermediate can be prepared by using the following synthetic scheme.

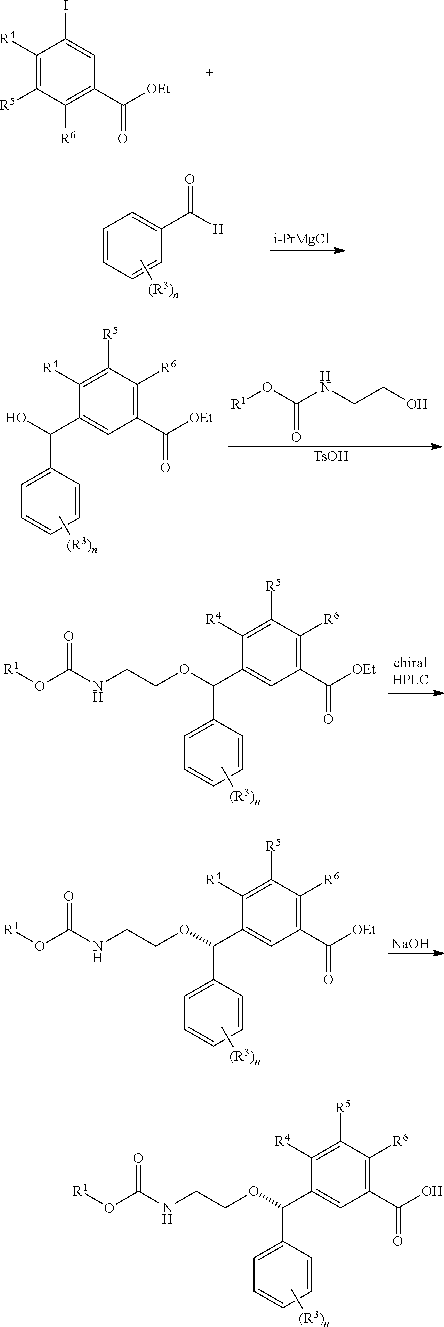

Alternatively, the benzoic acid intermediate can be prepared using the following synthetic scheme:

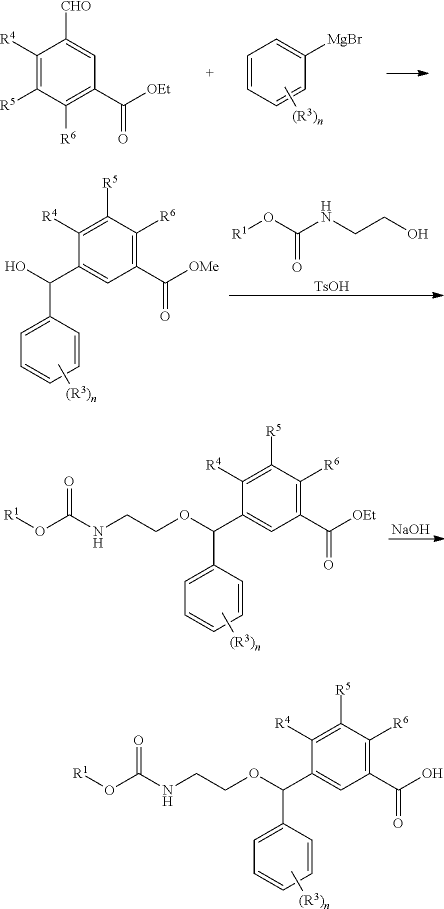

Alternatively, the benzoic acid intermediate can be prepared using the following synthetic scheme:

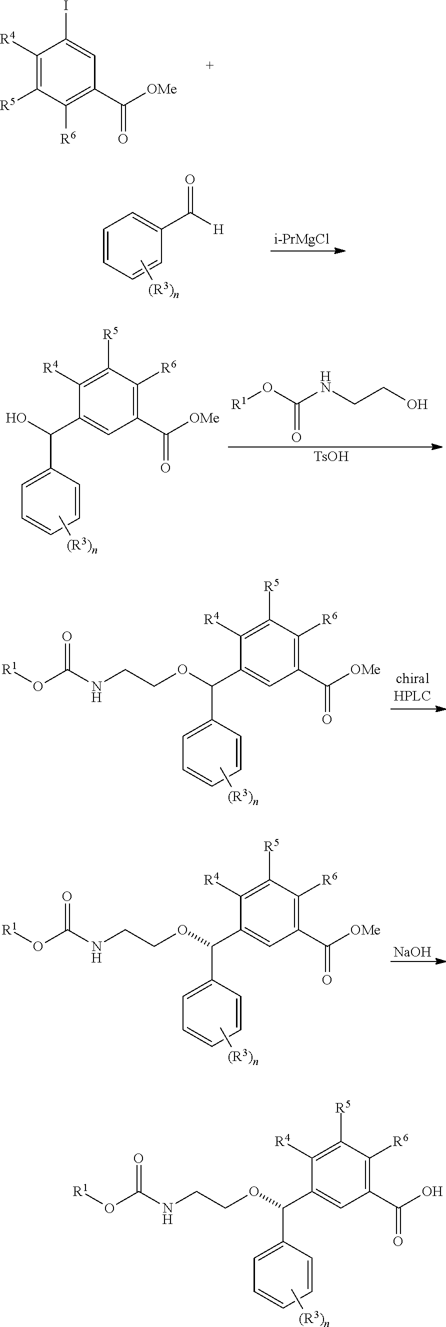

Alternatively, the benzoic acid intermediate can be prepared using the following synthetic scheme:

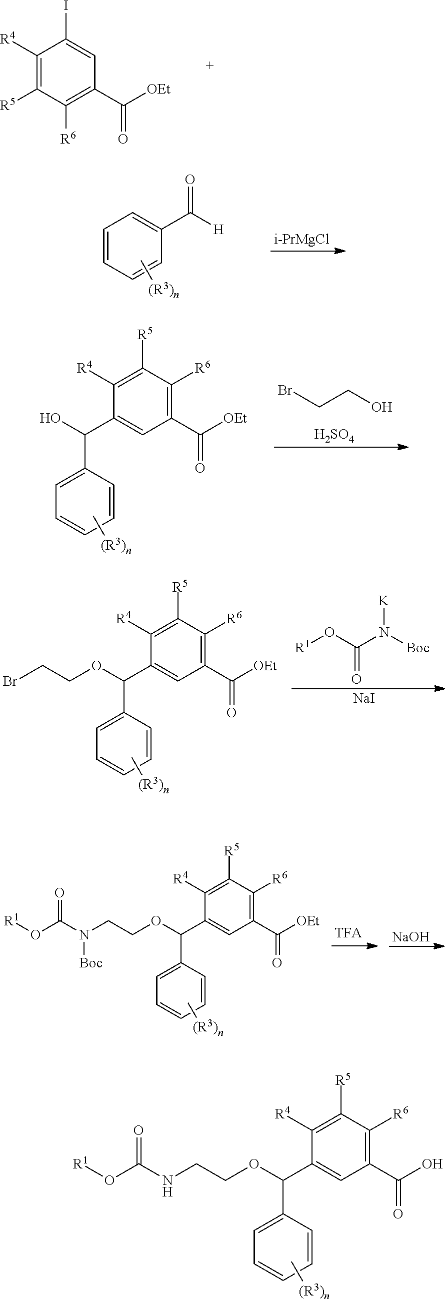

### Intermediate Preparation 1

### 2,2-dimethyl-4-(((R)-tetrahydro-2H-pyran-3-yl)methyl)oxazolidine

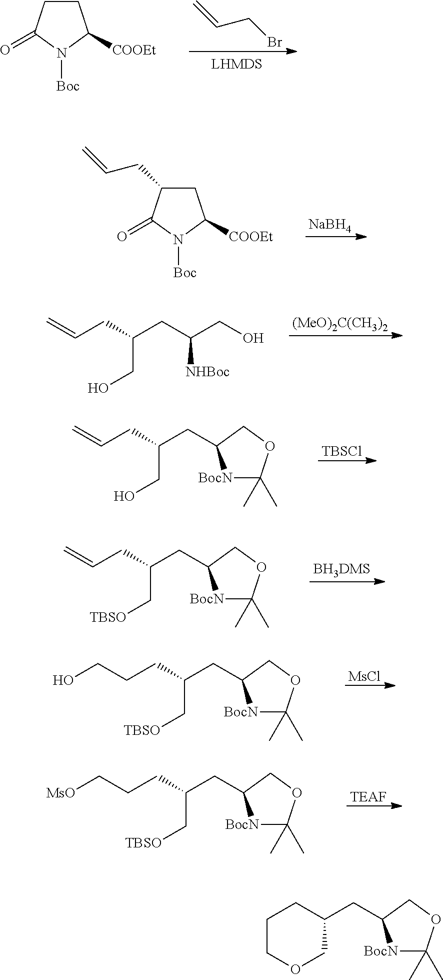

### Step 1. (2S,4R)-1-tert-butyl 2-ethyl 4-allyl-5-oxopyrrolidine-1,2-dicarboxylate

To a solution of HMDS in anhydrous THF (200 mL) was added dropwise 2.5 M n-BuLi in hexane (130 mL) and the mixture was stirred at −78° C. for 1 hr. To a solution of (S)-1-tert-butyl 2-ethyl 5-oxopyrrolidine-1,2-dicarboxylate (80 g, 0.311 mol) in anhydrous THF (1600 mL) stirred at −78° C. was added lithium hexamethyldisilazide in THF. After the reaction mixture was stirred at −78° C. for 1 hr, 3-bromopropene (38.47 g, 0.318 mol) in THF (200 mL) was added and stirring was continued for 2 hr. The reaction mixture was quenched with saturated ammonium chloride solution (600 mL) at −78° C. and extracted with EtOAc (3×500 mL). The combined organic layers were dried over Na₂SO₄, filtered and evaporated to dryness. The crude product was separated by column chromatography to afford (2S,4R)-1-tert-butyl 2-ethyl 4-allyl-5-oxopyrrolidine-1,2-dicarboxylate (15 g, 16%).

### Step 2. tert-butyl (2S,4R)-1-hydroxy-4-(hydroxymethyl)hept-6-en-2-ylcarbamate

To a solution of (2S,4R)-1-tert-butyl 2-ethyl 4-allyl-5-oxopyrrolidine-1,2-dicarboxylate (30 g, 0.1 mol) in MeOH/H₂O (700/70 mL) was added NaBH₄ (25 g, 0.66 mol), the result mixture was stirred 1 hr at rt and quenched with sat. aq. NH₄Cl (300 mL). The organic solvent was removed under vacuum and extracted with EtOAc (3×250 mL). The combined organic phases were washed with brine (250 mL) and dried over anhydrous Na₂SO₄, filtered and evaporated to afford crude tert-butyl (2S,4R)-1-hydroxy-4-(hydroxymethyl)hept-6-en-2-ylcarbamate (22 g, 85%). It was used in the next step without further purification.

### Step 3. (S)-tert-butyl 4-((R)-2-(hydroxymethyl)pent-4-enyl)-2,2-dimethyloxazolidine-3-carboxylate

To a solution of tert-butyl (2S,4R)-1-hydroxy-4-(hydroxymethyl)hept-6-en-2-ylcarbamate (6.8 g, 26.2 mmol) in acetone (150 mL), PTSA (0.45 g, 2.62 mmol) was added. The reaction mixture was cooled to −20° C. followed by the addition of 2,2-dimethoxypropane (4.1 g, 39.4 mmol). The resulting mixture was stirred and allowed to warm to rt for 1 hr. TEA (0.5 mL) was then added and stirred for another 5 min. The solvent was removed under reduced pressure. The residue was dissolved in Et₂O (300 mL), washed with 1 N HCl (80 mL), sat. aq. NaHCO₃ (80 mL), brine (80 mL) successively, and dried, filtered, and concentrated under vacuum to give crude (S)-tert-butyl 4-((R)-2-(hydroxymethyl)pent-4-enyl)-2,2-dimethyloxazolidine-3-carboxylate (7.5 g, 96%). It was used without further purification.

### Step 4. (S)-tert-butyl 4-((R)-2-((tert-butyldimethylsilyloxy)methyl)pent-4-enyl)-2,2-dimethyloxazolidine-3-carboxylate

To a solution of (S)-tert-butyl 4-((R)-2-(hydroxymethyl)pent-4-enyl)-2,2-dimethyloxazolidine-3-carboxylate (11.5 g, 38.4 mmol), imidazole (7.84 g, 115.2 mmol) and DMAP (234 mg, 1.92 mmol) in CH₂Cl₂ (200 mL) was added a solution of TBSCl (8.68 g, 57.6 mmol) in CH₂Cl₂ (100 mL) dropwise. The reaction mixture was stirred at rt for overnight. The reaction was washed with water (100 mL) and the aqueous layer was extracted with CH₂Cl₂ (3×100 mL), the combined organic layers was washed with brine (70 mL), then dried over Na₂SO₄, filtered and concentrated to give the crude product, which was purified by column chromatography to afford (S)-tert-butyl 4-((R)-2-((tert-butyldimethylsilyloxy)methyl)pent-4-enyl)-2,2-dimethyloxazolidine-3-carboxylate (9 g, 57%).

### Step 5. (S)-tert-butyl 4-((R)-2-((tert-butyldimethylsilyloxy)methyl)-5-hydroxypentyl)-2,2-dimethyloxazolidine-3-carboxylate

A solution of (S)-tert-butyl 4-((R)-2-((tert-butyldimethylsilyloxy)methyl)pent-4-enyl)-2,2-dimethyloxazolidine-3-carboxylate (26 g, 63 mmol) in THF (200 mL) was cooled in an ice-bath, followed by dropwise addition of 10 M BH₃.SMe₂ (6.3 mL). After stirring for 5 hr, 10% NaOH solution (32 mL) followed by 30% H₂O₂ (32 mL) were added carefully. The reaction mixture was stirred at rt for 16 hr. The reaction mixture was diluted with diethyl ether (500 mL) and the aqueous layer was extracted with diethyl ether (3×250 mL). The combined organic layers were washed with brine, dried over Na₂SO₄, filtered and concentrated to give the crude product, which was purified by column chromatography to afford (S)-tert-butyl 4-((R)-2-((tert-butyldimethylsilyloxy)methyl)-5-hydroxypentyl)-2,2-dimethyloxazolidine-3-carboxylate (19.6 g, 72%).

### Step 6. (S)-tert-butyl 4-((R)-2-((tert-butyldimethylsilyloxy)methyl)-5-(methylsulfonyloxy)pentyl)-2,2-dimethyloxazolidine-3-carboxylate

To a solution of (S)-tert-butyl 4-((R)-2-((tert-butyldimethylsilyloxy)methyl)-5-hydroxypentyl)-2,2-dimethyloxazolidine-3-carboxylate (32 g, 74.2 mmol) and Et₃N (22.5 g, 226 mmol) in CH₂Cl₂ (400 mL) was added a solution of MsCl (10.1 g, 89 mmol) in CH₂Cl₂ (50 mL) at 0-5° C. After addition, the reaction mixture was allowed to warm to rt and stir for 1 hr. The reaction was washed with water (200 mL) and the aqueous layer was extracted with CH₂Cl₂ (3×150 mL). The combined organic layers was washed with 10% citric acid (60 mL), sat. NaHCO₃ (60 mL) and brine (100 mL), then dried over Na₂SO₄, filtered and concentrated to give (S)-tert-butyl 4-((R)-2-((tert-butyldimethylsilyloxy)methyl)-5-(methylsulfonyloxy)pentyl)-2,2-dimethyloxazolidine-3-carboxylate (37.7 g, 100%), which was used in the next step without purification.

### Step 7. (S)-tert-butyl 2,2-dimethyl-4-(((R)-tetrahydro-2H-pyran-3-yl)methyl)oxazolidine-3-carboxylate

To a solution of (S)-tert-butyl 4-((R)-2-((tert-butyldimethylsilyloxy)methyl)-5-(methylsulfonyloxy)pentyl)-2,2-dimethyloxazolidine-3-carboxylate (37.7 g, 74.2 mmol) in THF (1000 mL) was added tetraethylammonium fluoride hydrate (41 g, 185.5 mmol) in portions. The reaction mixture was stirred under reflux overnight. The mixture was diluted with EtOAc (1000 mL), washed with water (300 mL) and brine (500 mL). The organic phase was dried over Na₂SO₄, filtered and concentrated in vacuo to give the crude product, which was purified by column chromatography to afford (S)-tert-butyl 2,2-dimethyl-4-(((R)-tetrahydro-2H-pyran-3-yl)methyl)oxazolidine-3-carboxylate (12.0 g, 54%).

### Intermediate Preparation 2

### tert-butyl (S)-1-amino-3-((R)-tetrahydro-2H-pyran-3-yl)propan-2-ylcarbamate

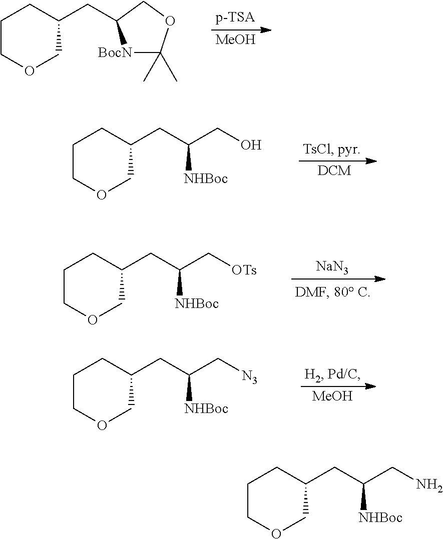

### Step 1. Preparation of tert-butyl (S)-1-hydroxy-3-((R)-tetrahydro-2H-pyran-3-yl)propan-2-ylcarbamate

To a solution of (S)-tert-butyl 2,2-dimethyl-4-(((R)-tetrahydro-2H-pyran-3-yl)methyl)oxazolidine-3-carboxylate (643 mg, 2.15 mmol) in MeOH (10 mL) was added p-TSA (37 mg, 0.22 mmol), then the solution was stirred at rt for 12 hr. TEA (2 mL) was added, followed by Boc₂O (46 mg, 0.21 mmol). After the addition the reaction solution was stirred for another 30 min. The organic solvent was removed under reduced pressure to give the crude product tert-butyl (S)-1-hydroxy-3-((R)-tetrahydro-2H-pyran-3-yl)propan-2-ylcarbamate. It was used in the next step without further purification. MS ESI+ve m/z 260 (M+1).

### Step 2. Preparation of (S)-2-(tert-butoxycarbonylamino)-3-((R)-tetrahydro-2H-pyran-3-yl)propyl 4-methylbenzenesulfonate

The above crude product tert-butyl (S)-1-hydroxy-3-((R)-tetrahydro-2H-pyran-3-yl)propan-2-ylcarbamate was dissolved in anhydrous DCM (22 mL). To this solution was added pyridine (2 mL) and TsCl (1.230 g, 6.45 mmol). After stirred at rt for 4 hr, another batch of pyridine (3 mL) and TsCl (0.700 g, 3.67 mmol) was added and stirred for another 12 hr. The reaction mixture was diluted with EtOAc (80 mL), washed with 1 N HCl (75 mL), followed by H₂O (2×30 mL), saturated aq. NaHCO₃, brine, and dried over anhydrous Na₂SO₄, and filtered, and concentrated under reduced pressure. The resulted slurry was purified through flash chromatography on silica gel (eluted with gradient system: 0-35% EtOAc in hexane) to afford (S)-2-(tert-butoxycarbonylamino)-3-((R)-tetrahydro-2H-pyran-3-yl)propyl 4-methylbenzenesulfonate, 670 mg, yield 75% for two steps. MS ESI+ve m/z 436 (M+Na).

### Step 3. tert-butyl (S)-1-azido-3-((R)-tetrahydro-2H-pyran-3-yl)propan-2-ylcarbamate

The solution of (S)-2-(tert-butoxycarbonylamino)-3-((R)-tetrahydro-2H-pyran-3-yl)propyl 4-methylbenzenesulfonate (132 mg, 0.32 mmol) and NaN₃ (62 mg, 0.95 mmol) in anhydrous DMF was heated to 80° C. under N₂ atmosphere for 1.5 hr, cooled to rt and diluted with EtOAc, and washed with H₂O (3×20 mL), followed by brine, and dried over anhydrous Na₂SO₄, and filtered, and concentrated under reduced pressure. The resulted slurry was purified through flash chromatography on silica gel (eluted with gradient system: 0-30% EtOAc in hexane) to afford tert-butyl (S)-1-azido-3-((R)-tetrahydro-2H-pyran-3-yl)propan-2-ylcarbamate 58 mg, yield 64%. MS ESI+ve m/z 307 (M+Na).

### Step 4: tert-butyl (S)-1-amino-3-((R)-tetrahydro-2H-pyran-3-yl)propan-2-ylcarbamate

Hydrogenation of tert-butyl (S)-1-azido-3-((R)-tetrahydro-2H-pyran-3-yl)propan-2-ylcarbamate (146 mg, 0.51 mmol) was carried out in MeOH (10 mL), 10% Pd/C (25 mg) under 40 psi of H₂ for 2 h. After filtration 114 mg of tert-butyl (S)-1-amino-3-((R)-tetrahydro-2H-pyran-3-yl)propan-2-ylcarbamate was obtained, yield 86%. MS ESI+ve m/z 259 (M+H).

### Intermediate Preparation 3

### tert-butyl (S)-1-amino-3-((R)-tetrahydro-2H-pyran-3-yl)propan-2-yl(methyl)carbamate

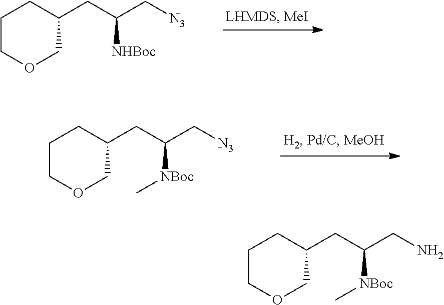

### Step 1. tert-butyl (S)-1-azido-3-((R)-tetrahydro-2H-pyran-3-yl)propan-2-yl(methyl)carbamate

To a solution of tert-butyl (S)-1-azido-3-((R)-tetrahydro-2H-pyran-3-yl)propan-2-ylcarbamate (30 mg, 0.11 mmol) in anhydrous THF (4 mL) at −78° C. was added 1.0 M LHMDS solution in THF (253 μL, 0.25 mmol), then stirred at this temperature for 30 min. To this mixture was added MeI (125 μL, 0.22 mmol), then the temperature was allowed to warm to 0° C., and stand for 12 hr in the refrigerator. The reaction mixture was quenched with saturated aq. NH₄Cl, extracted with EtOAc (30 mL), the separated organic phase was washed with H₂O (2×10 mL), brine, and dried (Na₂SO₄), and filtered. The filtrate was concentrated, the resulting slurry was purified through flash chromatography on silica gel (eluted with gradient system, 0-30% EtOAc in hexane) to afford tert-butyl (S)-1-azido-3-((R)-tetrahydro-2H-pyran-3-yl)propan-2-yl(methyl)carbamate 31 mg, yield 100%. MS ESI+ve m/z 321 (M+Na).

### Step 2. tert-butyl (S)-1-amino-3-((R)-tetrahydro-2H-pyran-3-yl)propan-2-yl(methyl)carbamate

Hydrogenation of (S)-1-azido-3-((R)-tetrahydro-2H-pyran-3-yl)propan-2-yl(methyl)carbamate (62 mg, 0.51 mmol) was carried out in EtOAc (20 mL), 10% Pd/C (15 mg) under 40 psi of H₂ for 2 h. After filtration 52 mg of tert-butyl (S)-1-amino-3-((R)-tetrahydro-2H-pyran-3-yl)propan-2-ylcarbamate was obtained, yield 91%. MS ESI+ve m/z 273 (M+H).

### Intermediate Preparation 4 tert-butyl (S)-1-amino-3-((R)-tetrahydro-2H-pyran-3-yl)propan-2-yl(methyl)carbamate

Alternatively, tert-butyl (S)-1-amino-3-((R)-tetrahydro-2H-pyran-3-yl)propan-2-yl(methyl)carbamate may be prepared by the following procedures:

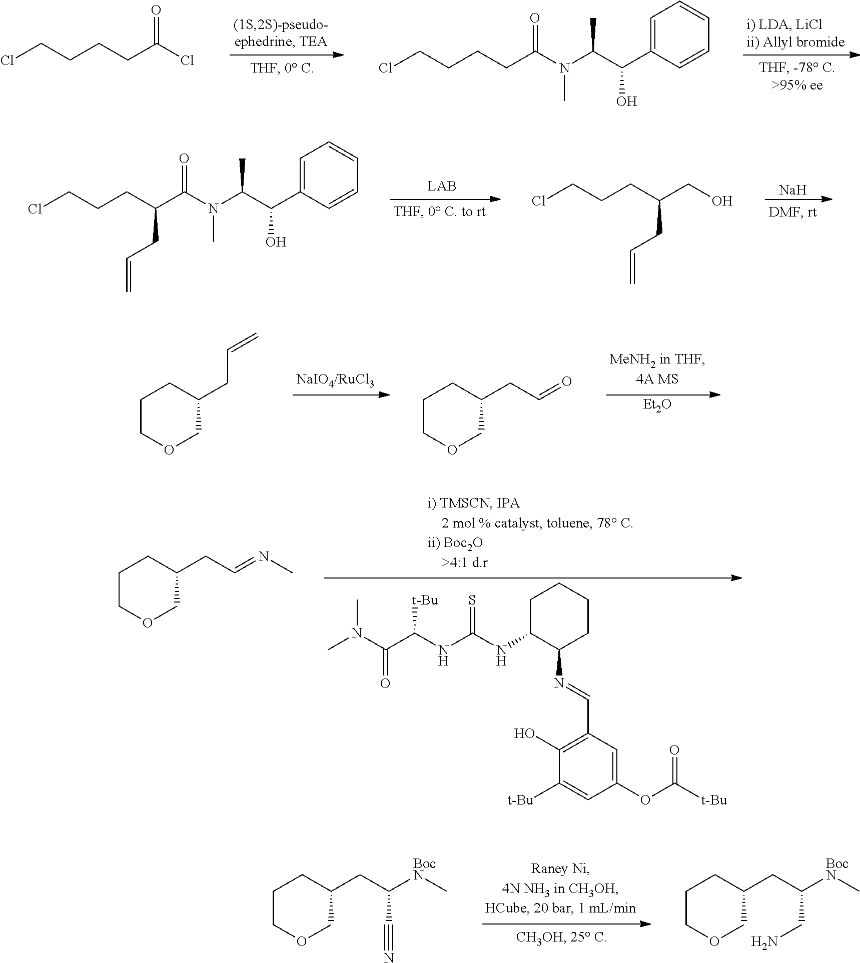

### Step 1. 5-Chloro-N-((1S,2S)-1-hydroxy-1-phenylpropan-2-yl)-N-methylpentanamide

To a magnetically stirred solution of (1S,2S)-pseudoephedrine (60 g, 363.1 mmol) in THF (600 mL) at room temperature was added triethylamine (65.4 mL, 472 mmol) in one portion. The resulting white suspension was cooled to 0° C. A solution of 5-chloropentanoyl chloride (49 mL, 381 mmol) in THF (130 mL) was added dropwise to the mixture over 45 min using an addition funnel. The mixture was then allowed to stir at 0° C. for 30 min. H₂O (40 mL) was added and the resulting mixture was concentrated to ˜10% of the original volume. The resulting solution was partitioned between H₂O/EtOAc and the layers were separated. The aqueous layer was extracted with EtOAc (600 mL). The combined organic layers were washed with saturated aqueous NaHCO₃, brine, dried over MgSO₄, filtered, and concentrated under reduced pressure to furnish the crude product as pale yellow oil. The crude amide was purified by flash chromatography (ISCO; 3×330 g column; CH₂Cl₂ to 5% MeOH/CH₂Cl₂) to provide the product as a clear, viscous oil. The residual MeOH was removed through azeotroping with toluene (3×100 mL) to provide 5-chloro-N-((1S,2S)-1-hydroxy-1-phenylpropan-2-yl)-N-methylpentanamide (96.2 g, 339 mmol, 93%). LCMS (m/z=266.0)

### Step 2. (R)-2-(3-Chloropropyl)-N-((1S,2S)-1-hydroxy-1-phenylpropan-2-yl)-N-methylpent-4-enamide

To a magnetically stirred suspension of LiCl (83 g, 1.96 mol) in THF (700 mL) at room temperature was added diisopropylamine (104 mL, 736 mmol) in one portion. n-BuLi (2.5M in hexane, 281 mL, 703 mmol) was added dropwise over 30 min using an addition funnel. The light yellow mixture stirred at −78° C. for 20 min and then was warmed to 0° C. for 15 min. The mixture was then cooled to −78° C. and 5-chloro-N-((1S,5S)-1-hydroxy-1-phenylpropan-2-yl)-N-methylpentanamide (92.8 g, 327 mmol) in THF (330 mL) was added dropwise over 30 min using an addition funnel. The mixture was stirred at −78° C. for 1 h and then was warmed to 0° C. for 25 min. Allylbromide (41.5 mL, 490 mmol) was then added slowly over 2 min via syringe and then the reaction was warmed to room temperature. The reaction stirred at room temperature for 50 min and was judged complete by LC/MS. The mixture was cooled to 0° C. and saturated aqueous NaHCO₃ (400 mL) and H₂O (200 mL) were added. EtOAc was added, the phases were separated and the aqueous phase was extracted with EtOAc (1500 mL total). The combined organic layers were washed with 1N HCl (4×150 mL), brine, dried over MgSO₄, filtered, and concentrated under reduced pressure to furnish (R)-2-(3-chloropropyl)-N-((1S,2S)-1-hydroxy-1-phenylpropan-2-yl)-N-methylpent-4-enamide as an orange oil (101.2 g, 312 mmol, 95%). The crude material was carried on without further purification. LC/MS (m/z=306.0).

### Step 3. (R)-2-(3-Chloropropyl)pent-4-en-1-ol

A magnetically stirred solution of diisopropylamine (184 mL, 1.29 mol) in THF (600 mL) was cooled to −78° C. n-BuLi (2.5M in hexane, 482 mL, 1.21 mol) was added dropwise over 35 min using an addition funnel. The cloudy mixture stirred at −78° C. for 15 min and then was warmed to 0° C. for 15 min during which time the solution became clear and light yellow. Borane-ammonia complex (90%, 42 g, 1.24 mol) was added in four equal portions, one minute apart. (Caution: vigorous evolution of gas). The cloudy mixture was warmed to room temperature for 20 min and then was recooled to 0° C. (R)-2-(3-chloropropyl)-N-((1S,2S)-1-hydroxy-1-phenylpropan-2-yl)-N-methylpent-4-enamide (100.2 g, 309 mmol) in THF (300 mL) was added dropwise over 10 min using an addition funnel. The reaction was warmed to room temperature and stirred for 2.5 h. The reaction was cooled to −10° C. and was quenched with HCl (3M, 1500 mL). The phases were separated and the aqueous phase was extracted with Et₂O (2000 mL total). The combined organic layers were washed with 3N HCl, brine, dried over MgSO₄, filtered, and concentrated under reduced pressure to furnish the crude product as a yellow oil. The crude material was purified by flash chromatography (ISCO; 330 g column; Hexane to 30% EtOAc/Hexane) to provide (R)-2-(3-chloropropyl)pent-4-en-1-ol as a clear, viscous oil (32.6 g, 200 mmol, 65%); ¹H NMR (400 MHz. CDCl₃) δ 5.82 (m, 1H), 5.07 (m, 2H), 3.78 (m, 1H), 3.58 (d, J=8.0 Hz, 2H), 3.54 (t, J=8 Hz, 2H), 2.14 (m, 2H), 1.85 (m, 2H), 1.64 (m, 1H), 1.49 (m, 1H).

### Step 4. (R)-3-Allyl-tetrahydro-2H-pyran

DMF (350 mL) was added to a round bottom flask containing NaH (60% w/w, 15 g, 0.376 mmol) and a magnetic stir bar. The suspension was cooled to 5-10° C. in an ice bath and stirred for 5 min. A solution of (R)-2-(3-chloropropyl)pent-4-en-1-ol (30.6 g, 188 mmol) in DMF (350 mL) was added via addition funnel over 25 min. Caution: Gas evolution and exotherm. The resulting creamy suspension was stirred for 30 min. The reaction was warmed to room temperature and the resulting beige suspension was stirred for 2 h, at which time it was judged complete by TLC. The reaction mixture was cooled to 0° C. and quenched by addition of H₂O (250 mL) and HCl (3N, 250 mL). The phases were separated and the aqueous phase was extracted with petroleum ether (4×250 mL). The combined with organic layers were washed with H₂O, brine, dried over MgSO₄, filtered, and concentrated under reduced pressure to furnish the crude product as a yellow oil. The crude material was purified by flash chromatography (ISCO; 120 g column; Hexane to 30% EtOAc/Hexane) to provide (R)-3-allyl-tetrahydro-2H-pyran as a clear oil (19.8 g, 157 mmol, 83%); ¹H NMR (400 MHz. CDCl₃) δ 5.72-5.82 (m, 1H), 5.00-5.06 (m, 2H), 3.86-3.91 (m, 2H), 3.37 (m, 1H), 3.08 (t, J=12 Hz, 1H), 1.85-1.98 (m, 3H), 1.59-1.69 (m, 3H), 1.15-1.21 (m, 1H).

### Step 5. (R)-2-(Tetrahydro-2H-pyran-3-yl)acetaldehyde

To a magnetically stirred solution of (R)-3-allyl-tetrahydro-2H-pyran (18.7 g, 148 mmol) in acetonitrile (740 mL) at room temperature was added RuCl₃.2H₂O (1.43 g, 5.92 mmol) in one portion. The resulting dark brown solution was stirred at room temperature for 5 min and then NaIO₄ (69 g, 326 mmol) was added in one portion. H₂O was added in small portions (10×8 mL) at 5 min intervals. The reaction was stirred at room temperature for 30 min, at which time it was judged complete by TLC. The reaction mixture was quenched by addition of saturated aqueous Na₂S₂O₃ (250 mL) and H₂O (1000 mL). The phases were separated and the aqueous phase was extracted with Et₂O (4×400 mL). The combined with organic layers were washed with H₂O, brine, dried over MgSO₄, filtered, and concentrated under reduced pressure to furnish the crude product as a yellow oil. The crude material was purified by flash chromatography (ISCO; 120 g column; Hexane to 40% EtOAc/Hexane) to provide (R)-2-(tetrahydro-2H-pyran-3-yl)acetaldehyde as a yellow oil (14.3 g, 111 mmol, 60%); ¹H NMR (400 MHz, CDCl₃) δ 9.78 (t, J=2, 1H), 3.84-3.88 (m, 2H), 3.40-3.47 (m, 1H), 3.17 (dd, J=11.2, 8.8 Hz, 1H), 2.31-2.41 (m, 2H), 2.21-2.28 (m, 1H), 1.88-1.93 (m, 1H), 1.61-1.72 (m, 2H), 1.29-1.33 (m, 1H).

### Step 6. (R,E)-N-(2-(Tetrahydro-2H-pyran-3-yl)ethylidene)methanamine

To a magnetically stirred solution of (R)-2-(tetrahydro-2H-pyran-3-yl)acetaldehyde (11 g, 85.8 mmol) in Et₂O (215 mL) at room temperature was added MeNH₂ (2M in THF, 215 mL, 429.2 mmol) and molecular sieves (4A, powdered, activated, 21.5 g). The reaction was stirred at room temperature for 1 h. The resulting mixture was then filtered and concentrated under reduced pressure to furnish (R,E)-N-(2-(tetrahydro-2H-pyran-3-yl)ethylidene)methanamine as a yellow oil (11.3 g, 80 mmol, 93%). The crude material was carried on without further purification. ¹H NMR (400 MHz, CDCl₃) δ 7.67 (m, 1H), 3.86-3.91 (m, 2H), 3.36-3.43 (m, 1H), 3.29 (s, 3H), 3.13 (dd, J=11.0, 9.8 Hz, 1H), 1.95-2.14 (m, 2H), 1.86-1.91 (m, 2H), 1.62-1.68 (m, 2H), 1.21-1.30 (m, 1H).

### Step 7. tert-Butyl (S)-1-cyano-2-((R)-tetrahydro-2H-pyran-3-yl)ethyl(methyl)-carbamate

A 2 L, round bottom flask was charged with toluene (400 mL), a magnetic stir bar, (R,E)-N-(2-(Tetrahydro-2H-pyran-3-yl)ethylidene)methanamine (11.3 g, 80.1 mmol) and 3-{(E)-[((1R,2R)-2-{[({(1S)-1-[(dimethylamino)carbonyl]-2,2-dimethylpropyl}amino)carbonothioyl]amino}cyclohexyl)imino]methyl}-5-(1,1-dimethylethyl)-4-hydroxyphenyl 2,2-dimethylpropanoate (J. Am. Chem. Soc., 2002, 124, 10012-10014) (0.9 g, 1.6 mmol). The mixture was cooled to −78° C. and trimethylsilanecarbonitrile (21.4 mL, 160.2 mmol) was added dropwise over 15 min using an addition funnel. Isopropyl alcohol (12.3 mL, 160.2 mmol) was then added dropwise over 10 min. The reaction stirred at −78° C. for 3 h and then was warmed to room temperature and stirred for 1 h. Bis(1,1-dimethylethyl) dicarbonate (35.0 g, 160.2 mmol) was then added and the resulting mixture was stirred at room temperature for 1 h. The reaction was quenched by the addition of saturated aqueous NaHCO₃ (400 mL) and EtOAc (300 mL). The layers were separated and the aqueous layer was washed with EtOAc (100 mL). The combined organic layers were dried over Na₂SO₄, filtered, and concentrated under reduced pressure to give the crude product. The crude material was divided into two parts and each was purified by flash chromatography (ISCO; 120 g column; 0% to 10% EtOAc/Hexane over 30 min, then 10% EtOAc/Hexane 47 min, then 10% to 20% EtOAc/Hexane over 2 min, then 20% EtOAc/Hexane for 11 min). The two purified batches were combined to provide tert-butyl (S)-1-cyano-2-((R)-tetrahydro-2H-pyran-3-yl)ethyl(methyl)carbamate (18.9 g, 70 mmol, 86%) as an orange oil. ¹H NMR (400 MHz, CDCl₃) δ 5.00 (brs, 1H), 3.83-3.90 (m, 2H), 3.42-3.48 (m, 1H), 3.19 (dd, J=11.3, 8.6, 1H), 2.92 (s, 3H), 1.85-1.95 (m, 1H), 1.60-1.82 (m, 5H), 1.50 (s, 9H), 1.28-1.33 (m, 1H).

### Step 8. tert-Butyl (S)-1-amino-3-((R)-tetrahydro-2H-pyran-3-yl)propan-2-yl(methyl)carbamate

tert-Butyl (S)-1-cyano-2-((R)-tetrahydro-2H-pyran-3-yl)ethyl(methyl)carbamate (397 mg, 4:1 mixture of diastereomers at the alpha-amino stereocenter) was dissolved in a solution of 4M NH₃ in MeOH (15 mL) and passed through a Raney-nickel cartridge (CatCart®, 50 mm) on an in-line hydrogenation apparatus (H-Cube) with the following settings: ambient temperature (14° C.), flow rate 1.0 mL/min, H₂ pressure 30 atm. The solution was recirculated so that the product solution was fed back into the apparatus. After thirty minutes, TLC analysis (1:9 MeOH/CH₂Cl₂, KMnO₄ stain) showed complete conversion of the starting material. After 60 min total reaction time, the solution was evaporated to yield 371 mg (92%) of tert-butyl (S)-1-amino-3-((R)-tetrahydro-2H-pyran-3-yl)propan-2-yl(methyl)carbamate as a clear, rose-colored oil. LC-MS (ELSD) m/z 273.6 (M+H)⁺.

### Intermediate Preparation 5

### 1,1-Dimethylethyl methyl {(1S)-2-({[(phenylmethyl)oxy]carbonyl}amino)-1-[(3R)-tetrahydro-2H-pyran-3-ylmethyl]ethyl}carbamate and 1,1-Dimethylethyl methyl {(1R)-2-({[(phenylmethyl)oxy]carbonyl}amino)-1-[(3R)-tetrahydro-2H-pyran-3-ylmethyl]ethyl}carbamate

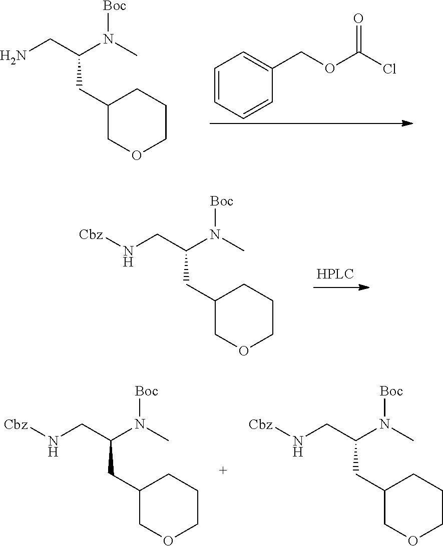

### Step 1. 1,1-Dimethylethyl methyl {2-({[(phenylmethyl)oxy]carbonyl}amino)-1-[(3R)-tetrahydro-2H-pyran-3-ylmethyl]ethyl}carbamate

To a 50 mL round-bottomed flask was added 1,1-dimethylethyl {2-amino-1-[(3R)-tetrahydro-2H-pyran-3-ylmethyl]ethyl}methylcarbamate (815 mg, 2.99 mmol) in dichloromethane (15 ml) to give a tan solution. The mixture was cooled to 0° C. (ice bath) and then N,N-diisopropylethylamine (1.045 ml, 5.98 mmol) and benzyl chloroformate (0.641 ml, 4.49 mmol) were added. After stirring for 3 hours at 0° C., the reaction was quenched with sat NH₄Cl (2 mL) and water (1 mL). The phases were separated and the organic layer was washed with sat NH₄Cl (2 mL). The aqueous layer was back extracted with CH₂Cl₂ (1×5 mL) and the combined organic layers and washed with saturated NaCl, dried over MgSO₄, filtered and concentrated to give 1.6 g of crude product as a reddish oil. The crude residue was purified by flash chromatography on silica gel {ISCO Combiflash, 40 g Analogix column, CH₂Cl₂/MeOH 0%->5%} and 1.10 g (dr 4:1) of 1,1-dimethylethyl methyl {2-({[(phenylmethyl)oxy]carbonyl}amino)-1-[(3R)-tetrahydro-2H-pyran-3-ylmethyl]ethyl}carbamate isolated as a reddish oil.

### Step 2. 1,1-Dimethylethyl methyl{(1S)-2-({[(phenylmethyl)oxy]carbonyl}amino)-1-[(3R)-tetrahydro-2H-pyran-3-ylmethyl]ethyl}carbamate and 1,1-Dimethylethyl methyl{(1R)-2-({[(phenylmethyl)oxy]carbonyl}amino)-1-[(3R)-tetrahydro-2H-pyran-3-ylmethyl]ethyl}carbamate

Purification via chiral HPLC [OD-H column (20×250 mm), 10/90 isopropanol/hexane with 0.1% diethylamine @ 10 mL/min] was necessary to separate the two diastereomers. The sample was dissolved in MeOH (10 mL), filtered and injected (16×). The combined fractions were collected and concentrated to give 1,1-dimethylethyl methyl{(1S)-2-({[(phenylmethyl)oxy]carbonyl}amino)-1-[(3R)-tetrahydro-2H-pyran-3-ylmethyl]ethyl}carbamate (732 mg, 1.765 mmol, 59.0% yield) (>99% de) as a pink oil (HPLC retention time of 7.46 min) and 95 mg of 1,1-dimethylethyl methyl {(1R)-2-({[(phenylmethyl)oxy]carbonyl}amino)-1-[(3S)-tetrahydro-2H-pyran-3-ylmethyl]ethyl}carbamate as a pink oil (HPLC retention time of 9.3 min). MS (m/z) 307.2 (M+H-Boc⁺).

### Intermediate Preparation 6

### 1,1-Dimethylethyl {(1S)-2-amino-1-[(3R)-tetrahydro-2H-pyran-3-ylmethyl]ethyl}methylcarbamate

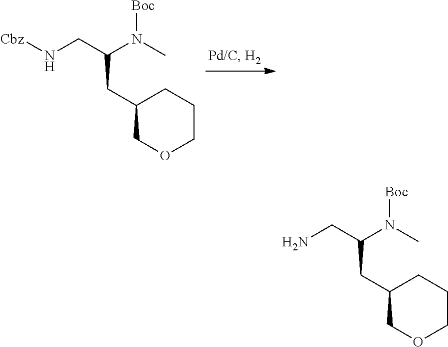

To a flask containing the 1,1-dimethylethyl methyl{(1S)-2-({[(phenylmethyl)oxy]carbonyl}amino)-1-[(3R)-tetrahydro-2H-pyran-3-ylmethyl]ethyl}carbamate and a large stirbar was added MeOH (10 mL). Palladium on carbon (0.093 g, 10% on carbon, 5 mol %) was added and a balloon of hydrogen affixed to the flask with a three-way valve. Very carefully, the contents of the flask were partially evacuated and refilled with N₂ several times while stirring, then partially evacuated and refilled with H₂ several times in such a way as to avoid bumping or excessive boiling. The hydrogenation was allowed to proceed at rt with vigorous stirring. After 1.5 h, TLC (5% MeOH/DCM) showed that the reaction was complete. The mixture was filtered through a pad of Celite and sand (cloudy, colorless), then through a 0.45 micron PTFE syringe filter (clear, colorless), and evaporated to yield 473.2 mg of 1,1-dimethylethyl {(1S)-2-amino-1-[(3R)-tetrahydro-2H-pyran-3-ylmethyl]ethyl}methylcarbamate as a clear slightly rose-colored heavy oil after drying in vacuo (100%).

### Intermediate Preparation 7

### 1,1-dimethylethyl {(1R)-2-amino-1-[(3R)-tetrahydro-2H-pyran-3-ylmethyl]ethyl}methylcarbamate

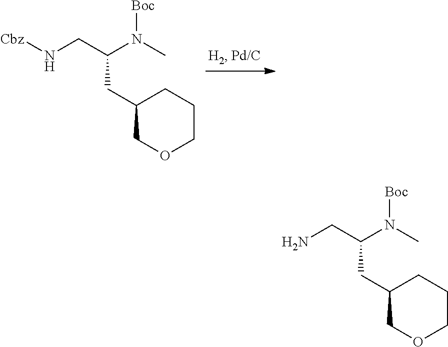

A solution of 1,1-dimethylethyl methyl{(1R)-2-({[(phenylmethyl)oxy]carbonyl}amino)-1-[(3R)-tetrahydro-2H-pyran-3-ylmethyl]ethyl}carbamate (0.175 g, 0.43 mmol) in 10 mL of MeOH was purged under nitrogen before it was charged with 10% Pd on carbon (0.023 g). The resulting mixture placed under a hydrogen balloon and was degassed three times and backfilled with hydrogen. The mixture was then maintained under hydrogen with stirring for 2 hours at room temperature. The crude material was filtered though a layer of celite under nitrogen and then though a 0.45 micron PTFE synringe filter to provide a clear solution which was concentrated to dryness to afford 1,1-dimethylethyl {(1R)-2-amino-1-[(3R)-tetrahydro-2H-pyran-3-ylmethyl]ethyl}methylcarbamate (0.08 g) as a colorless oil, which was used directly in the next reaction.

### Intermediate Preparation 8

### tert-butyl (S)-1-amino-3-((R)-tetrahydro-2H-pyran-3-yl)propan-2-yl(methyl)carbamate

Alternatively, tert-butyl (S)-1-amino-3-((R)-tetrahydro-2H-pyran-3-yl)propan-2-yl(methyl)carbamate may be prepared by the following procedures:

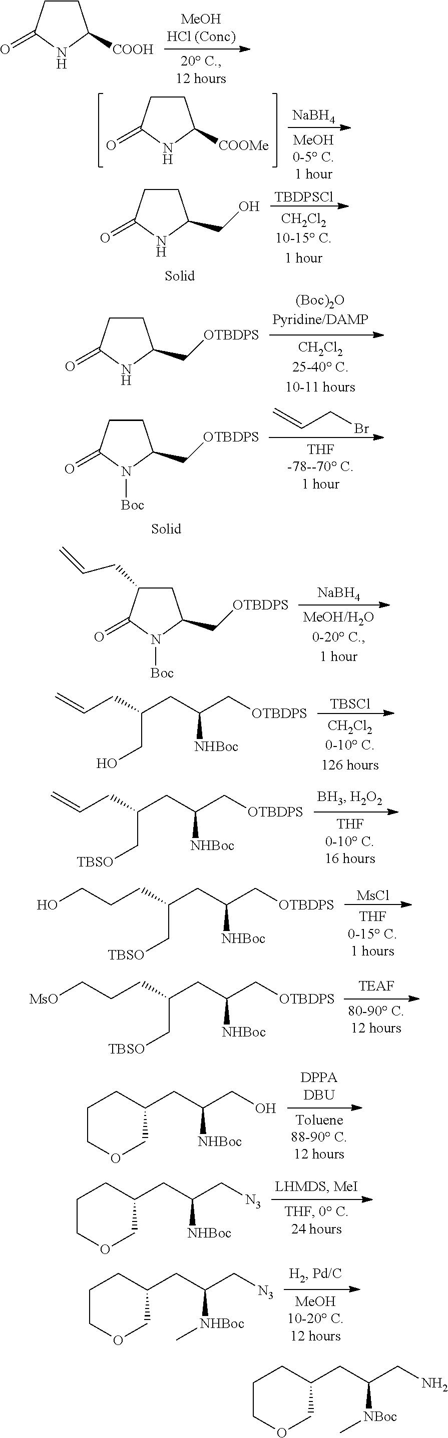

### Alternative Procedure:

Alternatively, tert-butyl (S)-1-amino-3-((R)-tetrahydro-2H-pyran-3-yl)propan-2-yl(methyl)carbamate may also be prepared by the following process where chiral hydrogenation catalysts may be used in a series of hydrogenation steps to provide enantiomerically enriched intermediates:

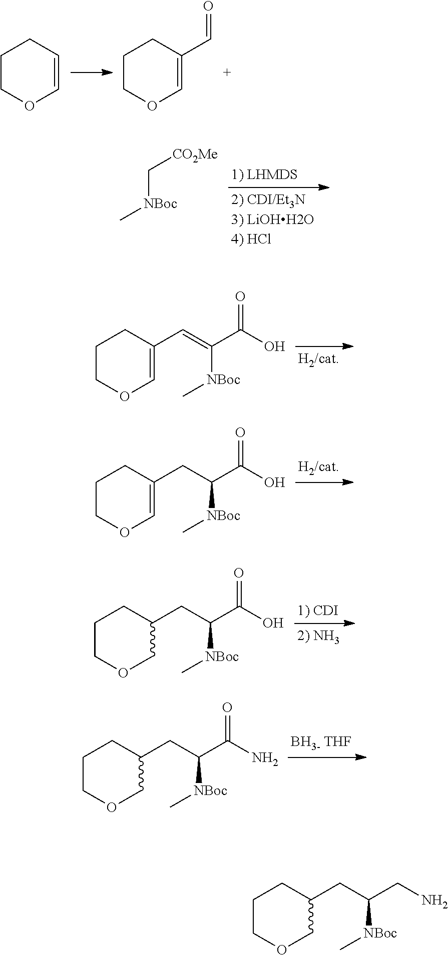

For example, hydrogenation of the dihydropyran-ene-amine to form the dihydropyran-amine may be accomplished in methanol, at 25° C., using about 88-110 psi hydrogen pressure, using 1-2 mol % of a catalyst generated from [Rh(nbd)₂]BF₄ and SL-M004-1 (SL-M004-1: (αR,αR)-2,2′-bis(α-N,N-dimethyl-aminophenylmethyl)-(S,S)-1,1′-bis[di(3,5-dimethyl-4-methoxyphenyl)phosphino]ferrocene, available from Solvias, Inc. Fort Lee, N.J.). Hydrogenation of the dihydropyran-amine to form the tetrahydropyran-amine may be accomplished at 50° C., using about 80 bar hydrogen pressure and 4 mol % catalyst loading of a catalyst generated from [Rh(COD)₂]O₃SCF₃ and SL-A109-2 (solvent: THF) or [Rh(nbd)₂]BF₄ and SL-A109-2 (solvent: methanol) (SL-A 109-2: (S)-(6,6′-dimethoxybiphenyl-2,2′-diyl)-bis[bis(3,5-di-tert-butyl-4-methoxyphenyl)phosphine], available from Solvias, Inc. Fort Lee, N.J.).

### Intermediate Preparation 9

### 1,1-Dimethylethyl methyl{(1S)-2-({[(phenylmethyl)oxy]carbonyl}amino)-1-[(3S)-tetrahydro-2H-pyran-3-ylmethyl]ethyl}carbamate and 1,1-Dimethylethyl methyl{(1R)-2-({[(phenylmethyl)oxy]carbonyl}amino)-1-[(3S)-tetrahydro-2H-pyran-3-ylmethyl]ethyl}carbamate

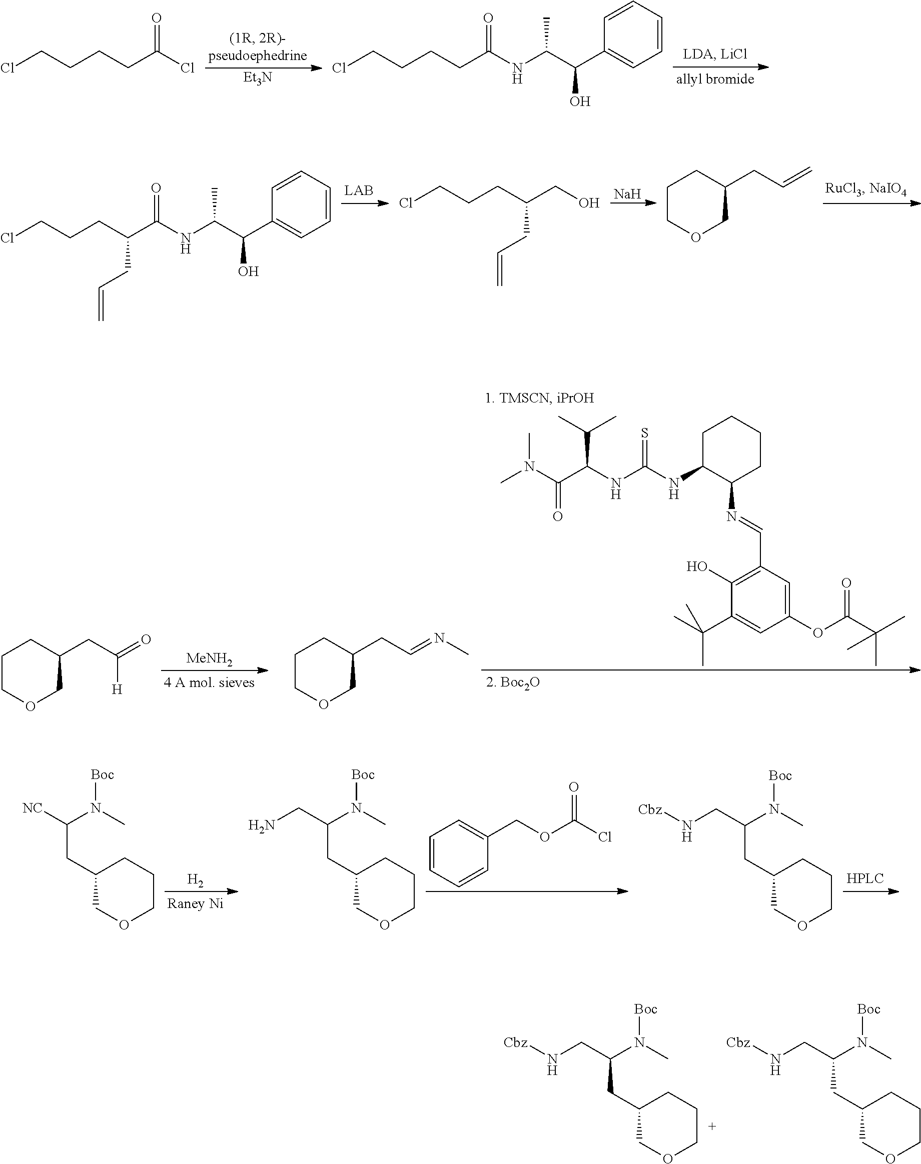

### Step 1. 5-Chloro-N-((1R,2R)-1-hydroxy-1-phenylpropan-2-yl)-N-methylpentanamide

5-Chloro-N-((1R,2R)-1-hydroxy-1-phenylpropan-2-yl)-N-methylpentanamide was prepared from 5-chloropentanoyl chloride (7.8 mL, 60.4 mmol) and (1R,2R)-pseudoephedrine (9.9 g, 60.4 mmol) according to the method described in Intermediate Preparation 4, Step 1.

### Step 2. (S)-2-(3-Chloropropyl)-N-((1R,2R)-1-hydroxy-1-phenylpropan-2-yl)-N-methylpent-4-enamide

(S)-2-(3-Chloropropyl)-N-((1R,2R)-1-hydroxy-1-phenylpropan-2-yl)-N-methylpent-4-enamide was prepared from 5-chloro-N-((1R,2R)-1-hydroxy-1-phenylpropan-2-yl)-N-methylpentanamide (17.7 g, 60.2 mmol) according to the method described in Intermediate Preparation 4, Step 2.

### Step 3. (5)-2-(3-Chloropropyl)pent-4-en-1-ol

(S)-2-(3-Chloropropyl)pent-4-en-1-ol was prepared from (S)-2-(3-chloropropyl)-N-((1R,2R)-1-hydroxy-1-phenylpropan-2-yl)-N-methylpent-4-enamide (18.2 g, 56.2 mmol) according to the method described in Intermediate Preparation 4, Step 3.

### Step 4. (3S)-3-(2-propen-1-yl)tetrahydro-2H-pyran

(3S)-3-(2-propen-1-yl)tetrahydro-2H-pyran was prepared from (S)-2-(3-chloropropyl)pent-4-en-1-ol (0.951 g, 5.84 mmol) according to the method described in Intermediate Preparation 4, Step 4.

### Step 5. (3S)-tetrahydro-2H-pyran-3-ylacetaldehyde

(3S)-tetrahydro-2H-pyran-3-ylacetaldehyde was prepared from (3S)-3-(2-propen-1-yl)tetrahydro-2H-pyran (4.5 g, 35.6 mmol) according to the method described in Intermediate Preparation 4, Step 5.

### Step 6. N-{(1E)-2-[(3S)-tetrahydro-2H-pyran-3-yl]ethylidene}methanamine

N-{(1E)-2-[(3S)-tetrahydro-2H-pyran-3-yl]ethylidene}methanamine was prepared from (3S)-tetrahydro-2H-pyran-3-ylacetaldehyde (2.75 g, 21.5 mmol) according to the method described in Intermediate Preparation 4, Step 6.

### Step 7. 1,1-Dimethylethyl {1-cyano-2-[(3S)-tetrahydro-2H-pyran-3-yl]ethyl}methylcarbamate

1,1-Dimethylethyl {1-cyano-2-[(3S)-tetrahydro-2H-pyran-3-yl]ethyl}methylcarbamate was prepared as a 3:1 mixture of diastereomers from N-{(1E)-2-[(35)-tetrahydro-2H-pyran-3-yl]ethylidene}methanamine (2.52 g, 17.8 mmol) according to the method described in Intermediate Preparation 4, Step 7.

### Step 8. 1,1-Dimethylethyl {2-amino-1-[(3S)-tetrahydro-2H-pyran-3-ylmethyl]ethyl}methylcarbamate

1,1-Dimethylethyl {2-amino-1-[(3S)-tetrahydro-2H-pyran-3-ylmethyl]ethyl}methylcarbamate was prepared from 1,1-dimethylethyl {1-cyano-2-[(3S)-tetrahydro-2H-pyran-3-yl]ethyl}methylcarbamate (3.75 g, 13.97 mmol) according to the method described in Intermediate Preparation 4, Step 8.

### Step 9. 1,1-Dimethylethyl methyl {2-({[(phenylmethyl)oxy]carbonyl}amino)-1-[(3S)-tetrahydro-2H-pyran-3-ylmethyl]ethyl}carbamate

1,1-Dimethylethyl methyl {2-({[(phenylmethyl)oxy]carbonyl}amino)-1-[(3S)-tetrahydro-2H-pyran-3-ylmethyl]ethyl}carbamate was prepared from 1,1-dimethylethyl {2-amino-1-[(3S)-tetrahydro-2H-pyran-3-ylmethyl]ethyl}methylcarbamate (3.71 g, 13.62 mmol) according to the method described in Intermediate Preparation 5, Step 1.

### Step 10. 1,1-Dimethylethyl methyl{(1S)-2-({[(phenylmethyl)oxy]carbonyl}amino)-1-[(3S)-tetrahydro-2H-pyran-3-ylmethyl]ethyl}carbamate and 1,1-Dimethylethyl methyl{(1R)-2-({[(phenylmethyl)oxy]carbonyl}amino)-1-[(3S)-tetrahydro-2H-pyran-3-ylmethyl]ethyl}carbamate

The diastereomers of 1,1-dimethylethyl methyl {2-(({[(phenylmethyl)oxy]carbonyl}amino)-1-[(3S)-tetrahydro-2H-pyran-3-ylmethyl]ethyl}carbamate were separated via chiral, preparative HPLC(OD-H column (20×250 mm) 20/80 isopropanol/hexane w/0.1% DEA @ 12 mL/min, Runtime −22 min). A 730 mg sample was dissolved in 7.5 mL methanol and then filtered. Another second sample (870 mg) was also dissolved in 8 mL methanol and then filtered. Approximately 196 mg were injected onto the column in a total of 11 injections. The fractions corresponding to the first peak (retention time of 4.45 min) were combined and concentrated to afford 1,1-dimethylethyl methyl{(1S)-2-({[(phenylmethyl)oxy]carbonyl}amino)-1-[(3S)-tetrahydro-2H-pyran-3-ylmethyl]ethyl}carbamate (1.31 g). The fractions corresponding to the second peak (retention time of 8.74 min) were combined and concentrated to provide 1,1-dimethylethyl methyl{(1R)-2-({[(phenylmethyl)oxy]carbonyl}amino)-1-[(3S)-tetrahydro-2H-pyran-3-ylmethyl]ethyl}carbamate (0.176 g).

### Intermediate Preparation 10

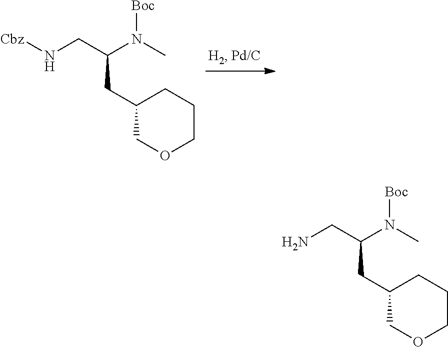

A solution of 1,1-dimethylethyl methyl{(1S)-2-({[(phenylmethyl)oxy]carbonyl}amino)-1-[(3S)-tetrahydro-2H-pyran-3-ylmethyl]ethyl}carbamate (1.31 g 3.22 mmol) in 25 ml of MeOH was purged under nitrogen and the flask charged with 10% Pd on carbon (0.171 g). The resulting mixture was fitted with a three-way adapter equipped with a hydrogen balloon. The flask was evacuated and backfilled three times with hydrogen and them maintained under a hydrogen atmosphere for 2 hours at room temperature. The crude material was filtered though a layer of celite under nitrogen and then though a 0.45 micron PTFE syringe filter and concentrated to afford 1,1-dimethylethyl {(1S)-2-amino-1-[(3S)-tetrahydro-2H-pyran-3-ylmethyl]ethyl}methylcarbamate (0.876 g), which was used in the next step without further purification

### Intermediate Preparation 11

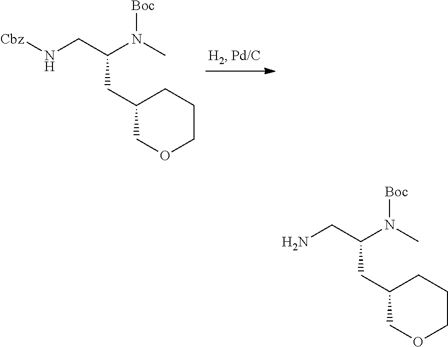

A solution of 1,1-dimethylethyl methyl{(1R)-2-({[(phenylmethyl)oxy]carbonyl}amino)-1-[(3S)-tetrahydro-2H-pyran-3-ylmethyl]ethyl}carbamate (0.176 g, 0.433 mmol) in 10 ml of MeOH was purged under nitrogen and the flask charged with 10% Pd on carbon (0.023 g). The resulting mixture was fitted with a three-way adapter equipped with a hydrogen balloon. The flask was evacuated and backfilled three times with hydrogen and them maintained under a hydrogen atmosphere for 2 hours at room temperature. The crude material was filtered though a layer of celite under nitrogen and then though a 0.45 micron PTFE syringe filter and concentrated to afford 1,1-dimethylethyl 1,1-dimethylethyl {(1R)-2-amino-1-[(3S)-tetrahydro-2H-pyran-3-ylmethyl]ethyl}methylcarbamate (0.120 g), which was used in the next step without further purification.

### Intermediate Preparation 12

### 1,1-Dimethylethyl [(1S)-2-azido-1-(cyclohexylmethyl)ethyl]methylcarbamate

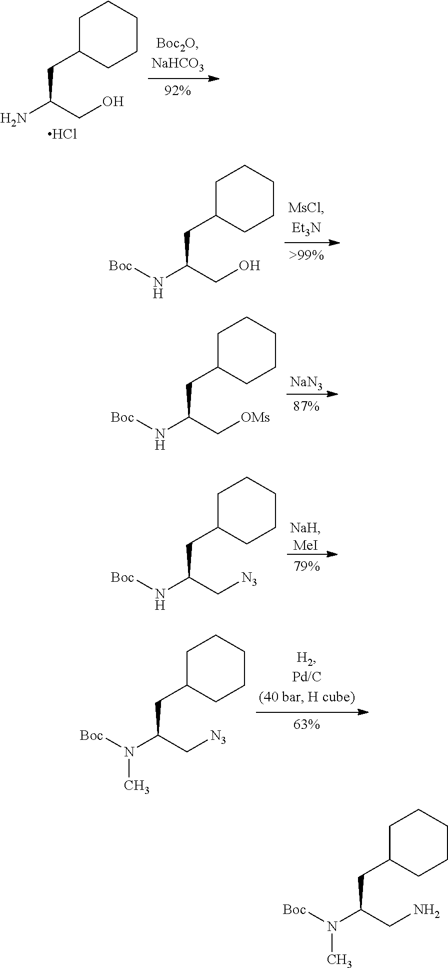

### Step 1. 1,1-dimethylethyl [(1S)-2-cyclohexyl-1-(hydroxymethyl)ethyl]carbamate

To a solution of (2S)-2-amino-3-cyclohexyl-1-propanol hydrochloride (5.0 g, 25.8 mmol) in dioxane (52 mL) and water (26 mL) at 0° C., sodium bicarbonate (2.16 g, 25.8 mmol) was added. Boc₂O then added in one portion. The resulting mixture was allowed to warm to room temperature and stir for 15 min before additional sodium bicarbonate (2.16 g, 25.8 mmol) was added. The mixture was then stirred overnight at room temperature. At this time the solvent was removed in vacuo and the residue taken up in ethyl acetate and water. The layers were separated and the aqueous layer extracted with ethyl acetate. The combined organics were then washed with brine, dried over Na₂SO₄, filtered, and concentrated in vacuo. The crude material was purified via column chromatography (ISCO, 40 g column, 0-20% ethyl acetate/methylene chloride) to give 6.08 g of 1,1-dimethylethyl [(1S)-2-cyclohexyl-1-(hydroxymethyl)ethyl]carbamate as a colorless oil (92%). MS (m/z) 258.6 (M+H⁺).

### Step 2. (2S)-3-Cyclohexyl-2-({[(1,1-dimethylethyl)oxy]carbonyl}amino)propyl methanesulfonate

To a solution of 1,1-dimethylethyl [(1S)-2-cyclohexyl-1-(hydroxymethyl)ethyl]carbamate (1.0 g, 3.89 mmol) and triethylamine (1.18 g, 11.7 mmol) in 16 mL of methylene chloride at 0° C., methanesulfonyl chloride (0.534 g, 4.66 mmol) was added. The resulting mixture was then warmed to rt and stirred for 50 min. The reaction mixture was washed the 0.1 N HCl, and the aqueous layer back-extracted with methylene chloride. The combined organic layers were then washed with saturated aqueous NaHCO₃, dried over Na₂SO₄, filtered and concentrated in vacuo to give 1.58 g (2S)-3-cyclohexyl-2-({[(1,1-dimethylethyl)oxy]carbonyl}amino)propyl methanesulfonate as a waxy yellow solid. The crude material was used in the next reaction without further purification. MS (m/z) 336.4 (M+H⁺).

### Step 3. 1,1-Dimethylethyl [(1S)-2-azido-1-(cyclohexylmethyl)ethyl]carbamate

To a solution of (2S)-3-cyclohexyl-2-({[(1,1-dimethylethyl)oxy]carbonyl}amino)propyl methanesulfonate (1.58 g, 3.89 mmol) in DMF (13 mL), sodium azide (1.26 g, 19.4 mmol) was added. The resulting mixture was then heated to 80° C. overnight. The mixture was then diluted was water and extracted with ether (3×). The combined organics were then washed with brine (3×), dried over Na₂SO₄, filtered and concentrated in vacuo. The crude product was purified via column chromatography (ISCO, 40 g cartride, 0-50% ethyl acetate/hexanes) to afford 0.945 g of 1,1-dimethylethyl [(1S)-2-azido-1-(cyclohexylmethyl)ethyl]carbamate as a colorless oil (87%). MS (m/z) 283.6 (M+H⁺).

### Step 4. 1,1-Dimethylethyl [(1S)-2-azido-1-(cyclohexylmethyl)ethyl]methylcarbamate

To a solution of 1,1-dimethylethyl [(1S)-2-azido-1-(cyclohexylmethyl)ethyl]carbamate (0.945 g, 3.35 mmol) in 17 mL of DMF at room temperature, sodium hydride (0.201 g, 5.02 mmol of a 60% dispersion in mineral oil) was added. Some gas evolution occurred and the solution turned yellow. Methyl iodide (0.312 mL, 5.02 mmol) then added and the resulting mixture stirred at room temperature for 1.5 h. The reaction mixture was quenched with 0.1 N HCl and partitioned between ether and water. The layers were separated and the aqueous layer backextracted with ether (2×). The combined organic layers were then washed with bring (3×), dried over Na₂SO₄, filtered and concentrated in vacuo. The crude product was purified via column chromatography (ISCO, 40 g cartridge, 0-50% ethyl acetate/hexanes), but still contained DMF. The crude material was then dissolved in ether, washed with brine (3×), dried over Na₂SO₄, filtered and concentrated in vacuo to provide 0.785 g of 1,1-dimethylethyl [(1S)-2-azido-1-(cyclohexylmethyl)ethyl]methylcarbamate as a colorless oil (79%). MS (m/z) 297.6 (M+H⁺).

The following diamine was prepared using procedures analogous to those described above substituting the indicated amino alcohol in Step 1:

| Diamine | Name | Amino Alcohol |
|---|---|---|
| 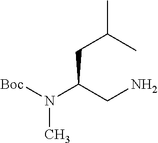 | 1,1-dimethylethyl [(1S)-1-(aminomethyl)- 3-methylbutyl] methylcarbamate | (2S)-2-amino-4- methyl-1-pentanol |

### Intermediate Preparation 13

### 1,1-dimethylethyl [(1S)-2-amino-1-(tetrahydro-2H-pyran-4-ylmethyl)ethyl]methylcarbamate

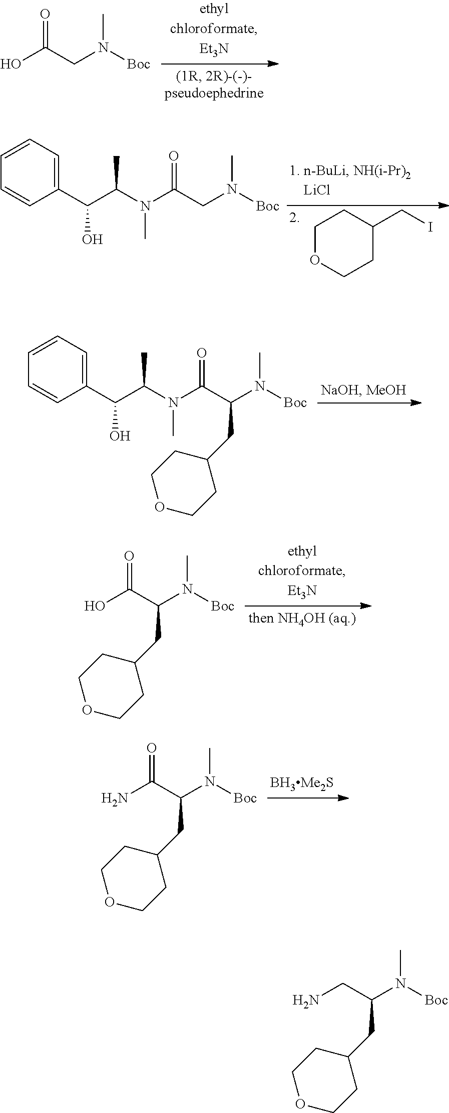

### Step 1. 1,1-dimethylethyl {2-[[(1R,2R)-2-hydroxy-1-methyl-2-phenylethyl](methyl)amino]-2-oxoethyl}methylcarbamate

To a solution of N-Boc-sarcosine (3.78 g, 20 mmol) and triethylamine (6.13 ml, 44 mmol) in THF (50 ml) at 0° C. was added ethyl chloroformate (1.91 mL, 20 mmol) to give a white suspension. The resulting suspension was stirred at 0° C. for 10 min and then warmed to rt for 2 h. The mixture was then recooled and (1R,2R)-(−)-pseudoephedrine (3.30 g, 20 mmol) was added and the resulting mixture was allowed to warm to room temperature and stir for 18 h. The reaction was concentrated and the residue dissolved in ethyl acetate and water (30 mL each). The layers were separated and the aqueous layer extracted with ethyl acetate (2×20 mL). The combined extracts were washed with HCl (1M, 20 mL), NaOH (1M, 20 mL), and brine, dried over MgSO₄, filtered and concentrated in vacuo, to give 5.07 g of crude material as a light amber oil. The product was purified via column chromatography (200 g silica gel 60, 230-400 mesh, 1-1.5% MeOH/CH₂Cl₂) to provide 1,1-dimethylethyl {2-[[(1R,2R)-2-hydroxy-1-methyl-2-phenylethyl](methyl)amino]-2-oxoethyl}methylcarbamate (2.77 g, 41.2%). MS (m/z) 337.0 (M+H⁺).

### Step 2. 1,1-dimethylethyl [(1S)-2-[[(1R,2R)-2-hydroxy-1-methyl-2-phenylethyl](methyl)amino]-2-oxo-1-(tetrahydro-2H-pyran-4-ylmethyl)ethyl]methylcarbamate

To a solution of diisopropylamine (2.19 ml. 15.36 mmol) in THF (20 mL) at −78° C., was added n-butyl lithium (6.46 ml, 2.5M in hexane, 16.15 mmol) dropwise. The resulting mixture was stirred at −78° C. for 30 min and was then added to a mixture of 1,1-dimethylethyl {2-[[(1R,2R)-2-hydroxy-1-methyl-2-phenylethyl](methyl)amino]-2-oxoethyl}methylcarbamate (2.65 g, 7.88 mmol) and lithium chloride (2.0 g, 47.3 mmol) via cannula at −23° C. The resulting mixture was stirred for 24 h and allowed to warm to room temperature before it was recooled in an ice bath and quenched with HCl (1M, 15.8 ml). The mixture was then extracted with EtOAc (3×20 ml) and the combined extracts washed with saturated NH₄Cl, brine, dried, filtered, and concentrated. This crude product was purified by column chromatography (160 g silica gel 60, 230-400 mesh, 25, 30, 40, then 50% EtOAc/hexanes) to provide 1,1-dimethylethyl [(1S)-2-[[(1R,2R)-2-hydroxy-1-methyl-2-phenylethyl](methyl)amino]-2-oxo-1-(tetrahydro-2H-pyran-4-ylmethyl)ethyl]methylcarbamate (510 mg, 95% pure and 1.2 g, 80% pure, 42% combined yield). MS (m/z) 435.2 (M+H⁺).

### Step 3. N-{[(1,1-dimethylethyl)oxy]carbonyl}-N-methyl-3-(tetrahydro-2H-pyran-4-yl)-L-alanine

To a solution of 1,1-dimethylethyl [(1S)-2-[[(1R,2R)-2-hydroxy-1-methyl-2-phenylethyl](methyl)amino]-2-oxo-1-(tetrahydro-2H-pyran-4-ylmethyl)ethyl]methylcarbamate (505 mg, 1.162 mmol) in methanol (20 mL), was added NaOH (5.81 ml, 1M). The resulting mixture was heated to reflux for 3 days. The reaction mixture was concentrated and the residue diluted with water (20 ml) and washed with ether (2×20 mL) and the combined ether washes were extracted with 0.5M NaOH (1×10 mL). The combined aqueous extracts were acidified with HCl (2M) to pH=1 and then extracted with EtOAc (2×50 ml). The combined organic extracts were washed with brine, dried, filtered and concentrated in vacuo to give N-{[(1,1-dimethylethyl)oxy]carbonyl}-N-methyl-3-(tetrahydro-2H-pyran-4-yl)-L-alanine (306 mg) as a clear oil, which was used in the next step without further purification. MS (m/z) 288.4 (M+H⁺).

### Step 4. 1,1-dimethylethyl [(1S)-2-amino-2-oxo-1-(tetrahydro-2H-pyran-4-ylmethyl)ethyl]methylcarbamate

To a solution of N-{[(1,1-dimethylethyl)oxy]carbonyl}-N-methyl-3-(tetrahydro-2H-pyran-4-yl)-L-alanine (296 mg, 1.03 mmol) and triethylamine (316 μl, 2.266 mmol) in THF (10 ml) at 0° C. was added ethyl chloroformate (98 μl, 1.03 mmol) to give a white suspension. The resulting suspension was stirred at 0° C. for 10 min and then warmed to rt for 2 h. The mixture was then recooled and ammonium hydroxide (0.5 ml) was added and the resulting mixture was allowed to warm to room temperature and stir for another 18 h. The reaction was concentrated and the residue diluted with ethyl acetate and water (10 mL each). The layers were separated and the aqueous layer extracted with ethyl acetate (2×10 mL). The combined organic extracts were washed with brine and dried over MgSO₄, filtered, and concentrated in vacuo to provide 1,1-dimethylethyl [(1S)-2-amino-2-oxo-1-(tetrahydro-2H-pyran-4-ylmethyl)ethyl]methylcarbamate (0.250 g), which was used in the next step without further purification. MS (m/z) 286.8 (M+H⁺).

### Step 5. 1,1-dimethylethyl [(1S)-2-amino-1-(tetrahydro-2H-pyran-4-ylmethyl)ethyl]methylcarbamate

To a refluxing solution of 1,1-dimethylethyl [(1S)-2-amino-2-oxo-1-(tetrahydro-2H-pyran-4-ylmethyl)ethyl]methylcarbamate (0.250 g, 0.873 mmol) in THF (10 ml) under argon was added borane dimethylsulfide complex (873 mL, 2M in THF, 1.75 mmol). The resulting mixture was heated at reflux for 2 h. After cooling to room temperature, the reaction mix was treated with KHSO₄ (600 mg) in water (6 ml), and the mixture was stirred at rt for 30 min. Excess NaOH (1N) was then added and the mixture extracted with ether. The ethereal extracts were washed with water, brine, dried, filtered, and concentrated. The crude material was purified via SCX column (loaded with methanol, washed with methanol and then eluted with 2M ammonia in methanol) to provide 1,1-dimethylethyl [(1S)-2-amino-1-(tetrahydro-2H-pyran-4-ylmethyl)ethyl]methylcarbamate (0.103 g, 43%). MS (m/z) 273.5 (M+H⁺).

### Intermediate Preparation 14

### 3-{(3-chlorophenyl)[(2-{[(methyloxy)carbonyl]amino}ethyl)oxy]methyl}benzoic acid

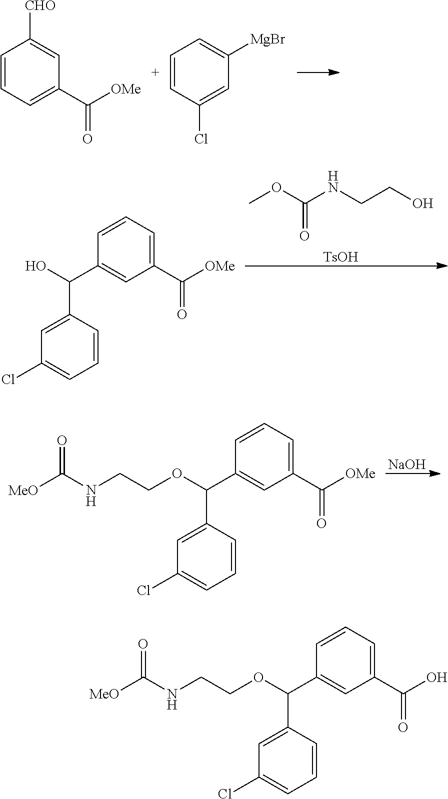

### Step 1. Methyl 3-[(3-chlorophenyl)(hydroxy)methyl]benzoate

To a solution of methyl-3-formylbenzoate (5 g, 30.5 mmol) in 70 mL of ether at 0° C., 3-chlorophenylmagnesium bromide (67 mL of a 0.5 M solution in THF, 33.5 mmol). After 1.5 h at 0° C., the reaction mixture was quenched by addition of saturated NaHCO₃ solution and water and the biphasic mixture was extracted with ethyl acetate. The combined organic layers were washed with brine, dried over MgSO₄, filtered and concentrated in vacuo to afford 9.2 g of a yellowish oil. This material was combined with 0.83 g of crude material (3.05 mmol of starting material) from a previous experiment and purified via column chromatography (ISCO; 10-100% ethyl acetate/hexanes) to afford 8.2 g of methyl 3-[(3-chlorophenyl)(hydroxy)methyl]benzoate (89% yield). MS (m/z) 277.3 (M+H⁺).

### Step 2. Methyl 3-{(3-chlorophenyl)[(2-{[(methyloxy)carbonyl]amino}ethyl)oxy]methyl}benzoate

To a solution of methyl 3-[(3-chlorophenyl)(hydroxy)methyl]benzoate (1.0 g, 3.6 mmol) and methyl (2-hydroxyethyl)carbamate (0.43 g, 3.6 mmol) in toluene p-toluenesulfonic acid (0.68 g, 3.6 mmol) was added. The resulting mixture was refluxed with a Dean-Stark trap for 1 h. The solvent was removed and the crude residue purified via column chromatography (ISCO, 5-100% ethyl acetate/hexanes) to give 0.270 g of methyl 3-{(3-chlorophenyl)[(2-{[(methyloxy)carbonyl]amino}ethyl)oxy]methyl}benzoate. MS (m/z) 378.4 (M+H⁺).

### Step 3. 3-{(3-chlorophenyl)[(2-{[(methyloxy)carbonyl]amino}ethyl)oxy]methyl}benzoic acid

To a solution of methyl 3-{(3-chlorophenyl)[(2-{[(methyloxy)carbonyl]amino}ethyl)oxy]methyl}benzoate (0.270 g, 0.7 mmol) in 3 mL of THF, sodium hydroxide (2.2 mL of a 2.5 N solution, 5.6 mmol) was added. The resulting mixture was stirred overnight at room temperature. The solvent was removed, the residue acidified with 1 N HCl, and extracted with ethyl acetate. The combined organics were then dried over MgSO4, filtered and concentrated in vacuo. This material was combined with that from another experiment (0.92 mmol of starting material) and purified via column chromatography (ISCO, 50-100% ethyl acetate/hexanes) to give 0.300 g of 3-{(3-chlorophenyl)[(2-{[(methyloxy)carbonyl]amino}ethyl)oxy]methyl}benzoic acid as a white solid (51% yield). MS (m/z) 364.5 (M+H⁺).

The following benzoic acid intermediates were prepared using procedures analogous to those described above substituting the indicated aldehyde for methyl-3-formybenzoate in Step 1.

| Benzoic Acid | Name | Aldehyde |
|---|---|---|
| 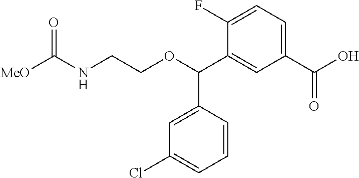 | 3-{(3-chlorophenyl)[(2- {[(methyloxy)carbonyl]amino} ethyl)oxy]methyl}-4- fluorobenzoic acid | methyl 4-fluoro-3- formylbenzoate |

### Intermediate Preparation 15

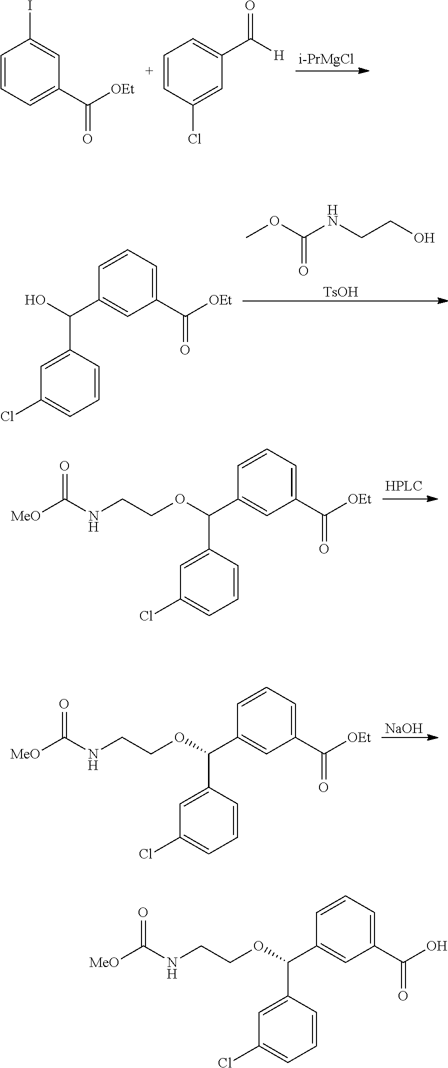

### Step 1. Ethyl 3-[(3-chlorophenyl)(hydroxy)methyl]benzoate

A 1 L 3-neck round bottom flask equipped with a 60 mL addition funnel was heated under vacuum with a heat gun. The vacuum line was replaced with a nitrogen line and a thermometer was added. Ethyl 3-iodobenzoate (18.29 ml, 109 mmol) was dissolved in tetrahydrofuran (THF) (362 ml). The mixture was cooled to −20 to −40° C. (dry ice/MeCN, monitored with internal thermometer) and isopropylmagnesium chloride in ether (59.8 ml, 120 mmol) was added dropwise using an addition funnel over 20 minutes. The reaction mixture was then stirred at −20 to −40° C. for 2.5 hours. 3-chlorobenzaldehyde (17.23 ml, 152 mmol) (dissolved in 40 mL of THF) was added over 20 minutes using a clean addition funnel. HPLC and TLC after one hour indicated that the iodide had been consumed. The mixture was warmed to 10° C. and 300 mL 1 N HCl was added carefully through an addition funnel followed by 200 mL of ethyl acetate. The layers were separated and the aqueous layer extracted with 50 mL EtOAc. The combined organic layers was dried over MgSO₄, filtered, and concentrated in vacuo. The crude oil was loaded directly onto a column and purified using silica gel chromatography (ISCO: 0-20% ethyl acetate/hexanes (30 min.), 20% (30 min.), 330 g silica) to afford 24.72 g of ethyl 3-[(3-chlorophenyl)(hydroxy)methyl]benzoate (˜95% pure, 74% yield). MS (m/z) 290.8 (M+H⁺).

### Step 2. Ethyl 3-{(3-chlorophenyl)[(2-{[(methyloxy)carbonyl]amino}ethyl)oxy]methyl}benzoate

Ethyl 3-[(3-chlorophenyl)(hydroxy)methyl]benzoate (1.63 g, 5.61 mmol), methyl (2-hydroxyethyl)carbamate (0.735 g, 6.17 mmol), and p-toluenesulfonic acid monohydrate (1.173 g, 6.17 mmol) were dissolved in toluene (56.1 ml) and heated to reflux with a Dean-Stark trap for 2 hours. The mixture was then cooled to room temperature and sat. NaHCO₃ (50 mL) and EtOAc (50 mL) added. The layers were separated and the organic layer was dried (Na₂SO₄), filtered, and concentrated in vacuo. The compound was loaded onto florisil and purified via silica gel chromatography (ISCO: 0-20% ethyl acetate/hexanes (30 min.), 20% (20 min.), 40 g silica) to give 0.557 g of ethyl 3-{(3-chlorophenyl)[(2-{[(methyloxy)carbonyl]amino}ethyl)oxy]methyl}benzoate (24% yield). MS (m/z) 391.8 (M+H⁺).

### Step 3. Ethyl 3-{(3-chlorophenyl)[(2-{[(methyloxy)carbonyl]amino}ethyl)oxy]methyl}benzoate

The enantiomers of ethyl 3-{(3-chlorophenyl)[(2-{[(methyloxy)carbonyl]amino}ethyl)oxy]methyl}benzoate were separated using a Chiralpack IB-H column (20×250 mm) 20/90 isopropanol/hexane w/0.1% DEA @ 15 mL/min. The sample (545 mg) was dissolved in 6 mL methanol, filtered and injected (12 injections total). Both, peaks were collected and checked by chiral HPLC. Peak. #2 (retention time of 8.921 min) was concentrated in vacuo to give 0.224 g (41%) of the desired enantiomer, ethyl 3-{(R)-(3-chlorophenyl)[(2-{[(methyloxy)carbonyl]amino}ethyl)oxy]methyl}benzoate. MS (m/z) 392.5 (M+H⁺). Peak #1 (retention time of 6.663 min) was also concentrated in vacuo to give 0.185 g of the undesired enantiomer, ethyl 3-{(S)-(3-chlorophenyl)[(2-{[(methyloxy)carbonyl]amino}ethyl)oxy]methyl}benzoate. MS (m/z) 392.5 (M+H⁺).

### Step 4. 3-{(R)-(3-chlorophenyl)[(2-{[(methyloxy)carbonyl]amino}ethyl)oxy]methyl}benzoic acid

To a round bottom flask containing 430 mg of ethyl 3-{(R)-(3-chlorophenyl)[(2-{[(methyloxy)carbonyl]amino}ethyl)oxy]methyl}benzoate was added MeOH and THF and fully dissolved. Then the 2.5M NaOH was added (stirred rapidly at rt in air). The cloudy reaction turned clear over ca. half hour. LCMS analysis after 1.5 h indicated that the starting material had been consumed. The reaction mixture was then quenched by slow addition of 1.0 N HCl until pH 1 was achieved, then diluted with water (50 mL), and extracted with EtOAc (4×50 mL). The combined EtOAc layers were washed with brine (1×50 mL), dried over Na₂SO₄ (overnight), and concentrated in vacuo to yield 384.2 mg of 3-{(R)-(3-chlorophenyl)[(2-{[(methyloxy)carbonyl]amino}ethyl)oxy]methyl}benzoic acid as a clear heavy colorless oil (˜100% yield). MS (m/z) 364.5 (M+H⁺).

The following benzoic acids were prepared using procedures to those analogous to those above substituting the indicated iodide for ethyl 3-iodobenzoate in Step 1. For racemic benzoic acids, Step 3 was omitted.

| Benzoic Acid | Name | Iodide |
|---|---|---|
| 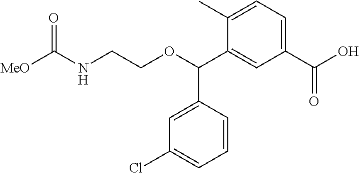 | 3-{(3-chlorophenyl)[(2- {[(methyloxy)carbonyl)amino} ethyl)oxy]methyl}-4- methylbenzoic acid | methyl 3-iodo- 4- methylbenzoate |
| 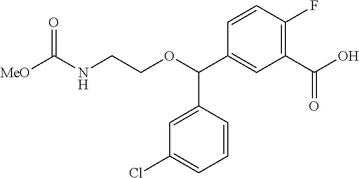 | 5-{(3-chlorophenyl)[(2- {[(methyloxy)carbonyl]amino} ethyl)oxy]methyl}-2- fluorobenzoic acid | methyl 2- fluoro-5- iodobenzoate |
| 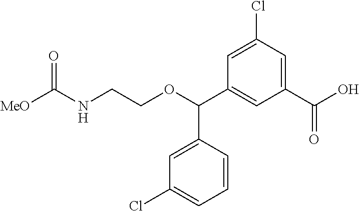 | 3-chloro-5-{(3-chlorophenyl)[(2- {[(methyloxy)carbonyl]amino} ethyl)oxy]methyl}benzoic acid | methyl 3- chloro-5- iodobenzoate |

### Intermediate Preparation 16

### 3-{(S)-(3-chlorophenyl)[(2-{[(methyloxy)carbonyl]amino}ethyl)oxy]methyl}-4-methylbenzoic acid

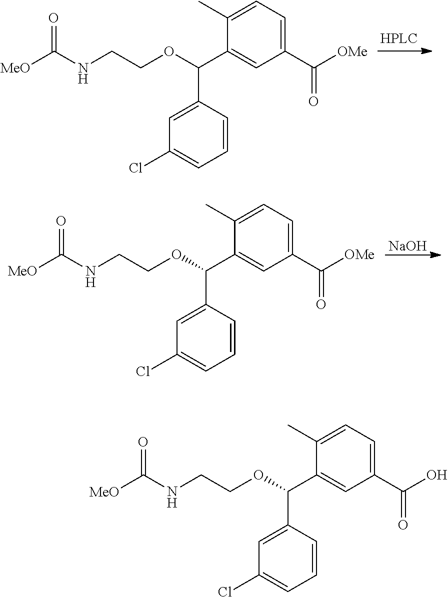

### Step 1. Methyl 3-{(S)-(3-chlorophenyl)[(2-{[(methyloxy)carbonyl]amino}ethyl)oxy]methyl}-4-methylbenzoate

The racemic sample, methyl 3-{(3-chlorophenyl)[(2-{[(methyloxy)carbonyl]amino}ethyl)oxy]methyl}-4-methylbenzoate, (0.433, 1.11 mmol) was dissolved in 4 mL, filtered and purified via preparative chiral HPLC (IB-H chiral column (20×250 mm), mobile phase 20% IPA/80% hexane with 0.1% diethylamine, 15 mL/min, 9 total injections). Fractions corresponding to the first peak (retention time of 6.592 min) were pooled and concentrated to afford methyl 3-{(S)-(3-chlorophenyl)[(2-{[(methyloxy)carbonyl]amino}ethyl)oxy]methyl}-4-methylbenzoate (0.173 g). Fractions corresponding to the second peak (retention time of 8.501 min) were combined and concentrated to give methyl 3-{(R)-(3-chlorophenyl)[(2-{[(methyloxy)carbonyl]amino}ethyl)oxy]methyl}-4-methylbenzoate (0.211 g).

### Step 2. 3-{(S)-(3-chlorophenyl)[(2-{[(methyloxy)carbonyl]amino}ethyl)oxy]methyl}-4-methylbenzoic acid

To a solution of methyl 3-{(S)-(3-chlorophenyl)[(2-{[(methyloxy)carbonyl]amino}ethyl)oxy]methyl}-4-methylbenzoate (0.17 g, 0.434 mmol) in methanol (4.34 mL) at room temperature, NaOH (1.735 mL, 1.735 mmol) was added. A white solid crashed out of solution, so THF (4.34 mL) was added to help solubility. The resulting mixture was stirred overnight at room temperature. The reaction was not complete, so an additional 2 equivalents of 1 N NaOH were added and the mixture stirred overnight. The methanol was removed in vacuo, the residue diluted with 5 mL of water, acidified to pH 3 with 1N HCl, and extracted with ethyl acetate (2×5 mL). The combined organic extracts were dried over MgSO₄, filtered, and concentrated. MS (m/z) 378.4 (M+H⁺).

### Intermediate Preparation 17

### 3-{(5-chloro-2-methylphenyl)[(2-{[(methyloxy)carbonyl]amino}ethyl)oxy]methyl}benzoic acid

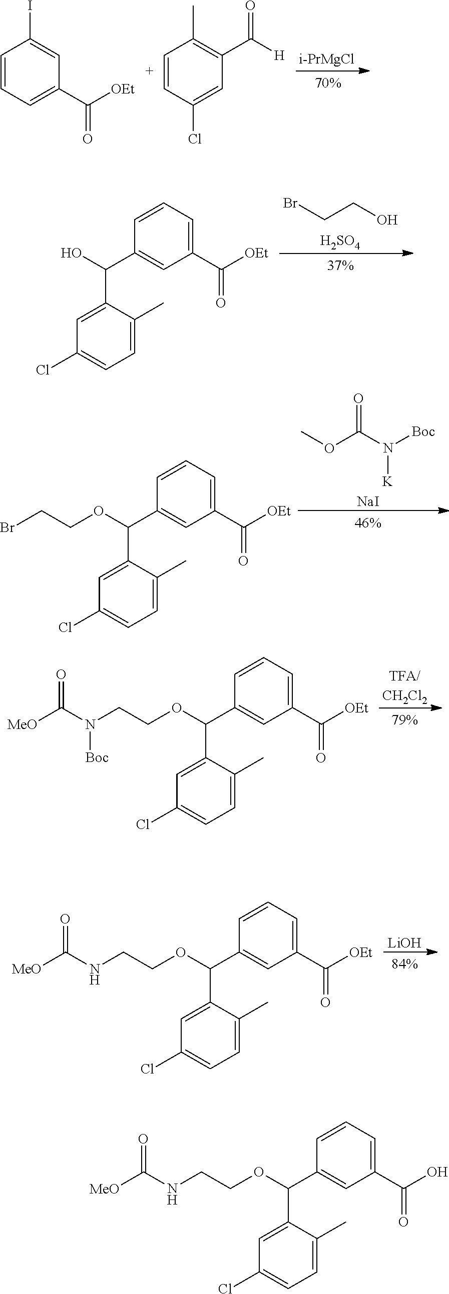

### Step 1. Ethyl 3-[(5-chloro-2-methylphenyl)(hydroxy)methyl]benzoate

To a solution of ethyl 3-iodobenzoate (13.16 g, 47.70 mmol) in THF at −30 to −40° C. isopropylmagnesium chloride (23.8 mL of a 2M solution, 47.70 mmol) was added dropwise. The resulting mixture was stirred for one hour before 5-chloro-2-methylbenzaldehyde (7.0 g, 45.3 mmol) was added. The reaction mixture was stirred at −30° C. for 30 min, then warmed to room temperature and stirred for an additional 10 min. Aqueous NH₄Cl and EtOAc were added and the layers separated. The organic layer was then washed with brine, dried over Na₂SO₄, filtered and concentrated in vacuo. The crude material was purified using silica gel chromatography to afford 10.2 g of ethyl 3-[(5-chloro-2-methylphenyl)(hydroxy)methyl]benzoate (70% yield).

### Step 2. Methyl 3-[[(2-bromoethyl)oxy](5-chloro-2-methylphenyl)methyl]benzoate

To 2-bromoethanol (33 mL, 469 mmol), was added methyl 3-[(5-chloro-2-methylphenyl)(hydroxy)methyl]benzoate (10.2 g, 33.5 mmol). After 5 min, sulfuric acid (10 drops) was added. The resulting mixture was then heated to 60 to 70° C. for 4 h before it was cooled to room temperature and diluted with ethyl acetate. The mixture was then washed with water, brine, dried over Na₂SO₄, filtered and concentrated in vacuo. The crude product was purified via column chromatography to give 5.1 g of methyl 3-[[(2-bromoethyl)oxy](5-chloro-2-methylphenyl)methyl]benzoate (37%).

### Step 3. Ethyl 3-{(5-chloro-2-methylphenyl)[(2-{{[(1,1-dimethylethyl)oxy]carbonyl}[(methyloxy)carbonyl]amino}ethyl)oxy]methyl}benzoate

To a solution of ethyl 3-[[(2-bromoethyl)oxy](5-chloro-2-methylphenyl)methyl]benzoate (5.1 g, 12.4 mmol) in acetone was added NaI (5.58 g, 37.2 mmol). The resulting mixture was then heated to 60° C. for 5 h before it was cooled to room temperature, filtered and washed with acetone. The acetone was removed and the residue diluted with ethyl acetate, washed with brine, dried over Na₂SO₄, filtered and concentrated in vacuo to afford the iodide intermediate. This material was dissolved in DMF and 1,1-dimethylethyl methyl ester potassium salt (3.97 g, 18.6 mmol) added. The mixture was then heated to 50-60° C. overnight before it was cooled to room temperature, and quenched with aqueous NH₄Cl and ethyl acetate. The layers were separated and the organic phase washed with brine, dried over Na₂SO₄, filtered, and concentrated in vacuo. The crude material was purified via column chromatography to give 2.9 g of ethyl 3-{(5-chloro-2-methylphenyl)[(2-{{[(1,1-dimethylethyl)oxy]carbonyl}[(methyloxy)carbonyl]amino}ethyl)oxy]methyl}benzoate as a pale yellow oil (46%).

### Step 4. Ethyl 3-{(5-chloro-2-methylphenyl)[(2-{[(methyloxy)carbonyl]amino}ethyl)oxy]methyl}benzoate

To a solution of TFA/CH₂Cl₂ was added ethyl 3-{(5-chloro-2-methylphenyl)[(2-{{[(1,1-dimethylethyl)oxy]carbonyl}[(methyloxy)carbonyl]amino}ethyl)oxy]methyl}benzoate (0.60 g, 1.19 mmol). The mixture was then stirred at room temperature for 20 minutes before the solvent was removed. The residue was diluted with ethyl acetate and washed with aqueous NaHCO₃, brine, dried over Na₂SO₄, filtered and concentrated in vacuo to give 0.380 g of ethyl 3-{(5-chloro-2-methylphenyl)[(2-{[(methyloxy)carbonyl]amino}ethyl)oxy]methyl}benzoate as a pale yellow oil (79%).

### Step 5. 3-{(5-chloro-2-methylphenyl)[(2-{[(methyloxy)carbonyl]amino}ethyl)oxy]methyl}benzoic acid

To a solution of ethyl 3-{(5-chloro-2-methylphenyl)[(2-{[(methyloxy)carbonyl]amino}ethyl)oxy]methyl}benzoate (0.380 g, 0.938 mmol) in methanol was added lithium hydroxide (0.225 g, 3.75 mmol) and water. The resulting mixture was heated to 40-50° C. for 2 h before it was cooled to room temperature and the solvent removed. The residue was dissolved in ethyl acetate and acidified to pH 2-3. The organic layer was then washed with brine, dried over Na₂SO₄, filtered and concentrated in vacuo to afford 0.300 g of 3-{(5-chloro-2-methylphenyl)[(2-{[(methyloxy)carbonyl]amino}ethyl)oxy]methyl}benzoic acid as a pale yellow solid (84%).

### Intermediate Preparation 18

### 3-{(R)-(5-chloro-2-methylphenyl)[(2-{[(methyloxy)carbonyl]amino}ethyl)oxy]methyl}benzoic acid

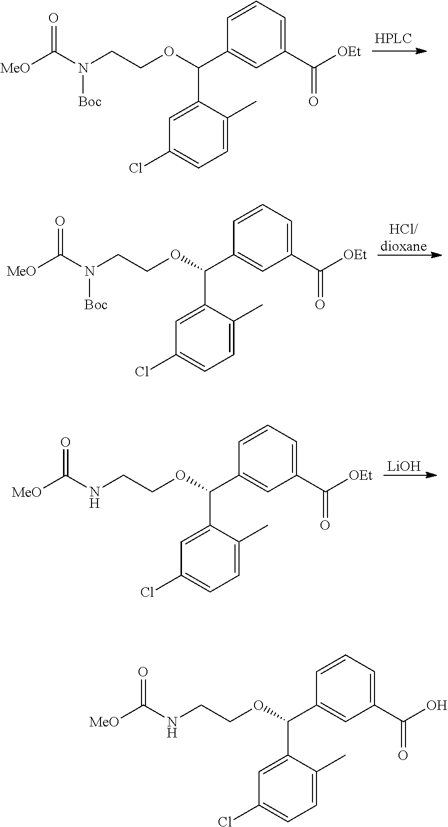

### Step 1. Ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate

The mixture of ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate and ethyl 3-{(S)-(5-chloro-2-methylphenyl)[(2-{{[(1,1-dimethylethyl)oxy]carbonyl}[(methyloxy)carbonyl]amino}ethyl)oxy]methyl}benzoate was separated via preparative chiral HPLC (OJ-H column (20×250 mm) 100% methanol @ 10 mL/min, Runtime −15 min). The sample (833 mg) was dissolved in 20 mL methanol and then filtered and processed using 5 individual injections and 12 stack injections (approximately 62.5 mg per injection). Fractions corresponding to the first peak (retention time of 4.729 min) were pooled and concentrated to afford ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate (0.287 g). Fractions corresponding to the second peak (retention time of 9.533 min) were also pooled and concentrated to provide ethyl 3-{(S)-(5-chloro-2-methylphenyl)[(2-{{[(1,1-dimethylethyl)oxy]carbonyl}[(methyloxy)carbonyl]amino}ethyl)oxy]methyl}benzoate (0.325 g).

### Step 2. Ethyl 3-{(R)-(5-chloro-2-methylphenyl)[(2-{[(methyloxy)carbonyl]amino}ethyl)oxy]methyl}benzoate

To a solution of ethyl 3-{(R)-(5-chloro-2-methylphenyl)[(2-{{[(1,1-dimethylethyl)oxy]carbonyl}[(methyloxy)carbonyl]amino}ethyl)oxy]methyl}benzoate (0.180 g, 0.36 mmol) in methylene chloride (10 mL), was added HCl (4M in dioxane, 3.56 mL). The resulting mixture was stirred at room temperature for 5 h. The solvent was removed in vacuo and the residue dissolved CH₂Cl₂ (10 mL) and washed with saturated NaHCO₃ (5 mL) and the aqueous was extracted with CH₂Cl₂ once more. The combined organic extracts were washed with brine, dried, filtered, and concentrated to afford ethyl 3-{(R)-(5-chloro-2-methylphenyl)[(2-{[(methyloxy)carbonyl]amino}ethyl)oxy]methyl}benzoate (0.140 g, 97%) as a clear oil, which was used in the next step without further purification. MS (m/z) 406.2 (M+H⁺).

### Step 3. 3-{(R)-(5-chloro-2-methylphenyl)[(2-{[(methyloxy)carbonyl]amino}ethyl)oxy]methyl}benzoic acid

To a solution of ethyl 3-{(R)-(5-chloro-2-methylphenyl)[(2-{[(methyloxy)carbonyl]amino}ethyl)oxy]methyl}benzoate (140 mg, 0.35 mmol) in THF (5 mL) and methanol (2 mL) was added LiOH (1M, 1.38 ml). The resulting mixture was stirred at rt for 16 h. The reaction was concentrated and the residue diluted with water (5 mL) and washed with EtOAc (5 mL). The aqueous layer was acidified with HCl (1N) to pH=2 and extracted with EtOAc (3×10 mL). The combined organic extracts were washed with brine, dried, filtered and concentrated in vacuo to give 3-{(R)-(5-chloro-2-methylphenyl)[(2-{[(methyloxy)carbonyl]amino}ethyl)oxy]methyl}benzoic acid (0.118 g, 91%) as a white solid. MS (m/z) 378.0 (M+H⁺).

Specific conditions for synthesizing the disclosed aspartic protease inhibitor compounds according to the above schemes are provided below.

### Example 1

Methyl {2-[((R)-(3-chlorophenyl){3-[({(2S)-2-(methylamino)-3-[(3R)-tetrahydro-2H-pyran-3-yl]propyl}amino)carbonyl]phenyl}methyl)oxy]ethyl}carbamate hydrochloride

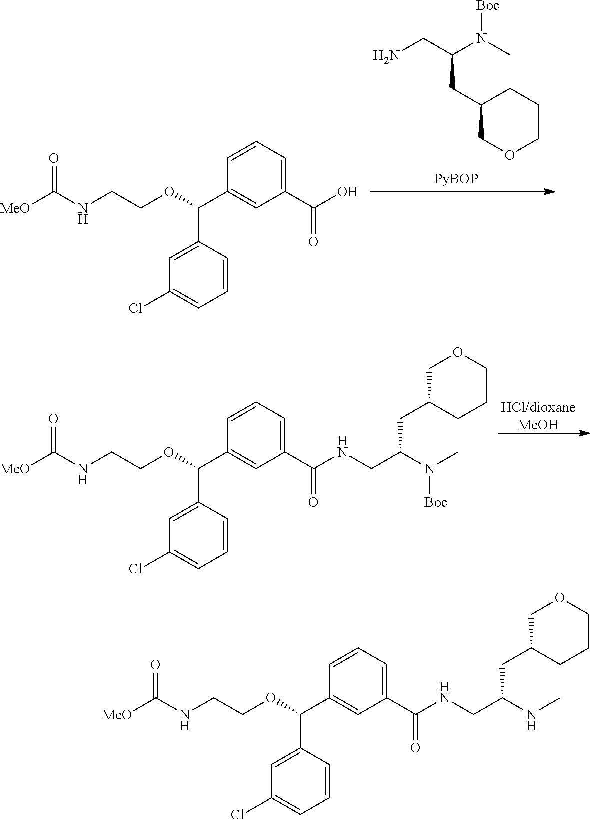

### Step 1. Methyl {2-[((R)-(3-chlorophenyl){3-[({(2S)-2-[{[(1,1-dimethylethyl)oxy]carbonyl}(methyl)amino]-3-[(3R)-tetrahydro-2H-pyran-3-yl]propyl}amino)carbonyl]phenyl}methyl)oxy]ethyl}carbamate

To a solution of 3-{(R)-(3-chlorophenyl)[(2-{[(methyloxy)carbonyl]amino}ethyl)oxy]methyl}benzoic acid (0.375 g, 1.031 mmol) in dichloromethane (10.31 ml) were added N,N-diisopropylethylamine (0.360 ml, 2.062 mmol), 1,1-dimethylethyl {(1S)-2-amino-1-[(3R)-tetrahydro-2H-pyran-3-ylmethyl]ethyl}methylcarbamate (0.309 g, 1.134 mmol), and PyBOP (0.590 g, 1.134 mmol). HPLC analysis after 1 hour indicated that the starting material had been consumed. The reaction mixture was concentrated, the crude material loaded onto florisil and purified using silica gel chromatography (ISCO: 30-75% ethyl acetate/hexanes (30 min.), 12 g silica) to give 0.68 g of methyl {2-[((R)-(3-chlorophenyl){3-[({(2S)-2-[{[(1,1-dimethylethyl)oxy]carbonyl}(methyl)amino]-3-[(3R)-tetrahydro-2H-pyran-3-yl]propyl}amino)carbonyl]phenyl}methyl)oxy]ethyl}carbamate that was 95% pure and contained a small amount of ethyl acetate by NMR (101% yield). MS (m/z) 618.6 (M+H⁺).

### Step 2. Methyl {2-[((R)-(3-chlorophenyl){3-[({(2S)-2-(methylamino)-3-[(3R)-tetrahydro-2H-pyran-3-yl]propyl}amino)carbonyl]phenyl}methyl)oxy]ethyl}carbamate hydrochloride

To a solution of methyl {2-[((R)-(3-chlorophenyl){3-[({(2S)-2-[{[(1,1-dimethylethyl)oxy]carbonyl}(methyl)amino]-3-[(3R)-tetrahydro-2H-pyran-3-yl]propyl}amino)carbonyl]phenyl}methyl)oxy]ethyl}carbamate (0.635 g, 1.027 mmol) in acetonitrile (10.27 ml) was added HCl in dioxane (1.284 ml, 5.14 mmol). The reaction mixture was concentrated and purified via HPLC (Agilent prep: 20-60% CH₃CN/H₂O, 0.1% TFA, 30×150 mm Sunfire C18, 25 mL/min, 15 min., 6 injections). The product fractions were concentrated on an EZ2 Genevac overnight. The product was then dissolved in EtOAc (30 mL) and 1 N NaOH (20 mL) added. The layers were separated and the aqueous layer extracted with EtOAc (2×10 mL). The combined organic layers were dried over MgSO₄, filtered, and concentrated to give 414 mg of methyl {2-[((R)-(3-chlorophenyl){3-[({(2S)-2-(methylamino)-3-[(3R)-tetrahydro-2H-pyran-3-yl]propyl}amino)carbonyl]phenyl}methyl)oxy]ethyl}carbamate (78% yield). The free base was then dissolved in 10 mL MeCN and added 0.4 mL of 4 N HCl/dioxane (2 eq. with respect to the 414 mg/0.8 mmol of free base) and concentrated. The material was azeotroped with additional acetonitrile and then MeOH and finally dissolved in 5 mL MeOH and filtered through Acrodisc CR 25 mm syringe filter with 0.2 um PTFE membrane to removed any particulate before it was concentrated to afford 0.570 g of methyl {2-[((R)-(3-chlorophenyl){3-[({(2S)-2-(methylamino)-3-[(3R)-tetrahydro-2H-pyran-3-yl]propyl}amino)carbonyl]phenyl}methyl)oxy]ethyl}carbamate hydrochloride as a white foam (71% yield). MS (m/z) 519.0 (M+H⁺). ¹H NMR (400 MHz, DMSO-d6) δ 8.80 (t, J=5.7 Hz, 1H), 8.72 (s, 1H), 7.93 (s, 1H), 7.84 (d, J=7.9 Hz, 1H), 7.59 (d, J=7.5 Hz, 1H), 7.49 (s, 1H), 7.47 (t, J=7.6 Hz, 1H), 7.39-7.29 (m, 4H), 5.57 (s, 1H), 3.74 (dd, J=11.0, 3.5 Hz, 2H), 3.61 (dt, J=14.7, 4.4 Hz, 1H), 3.51 (s, 3H), 3.46 (dd, J=14.9, 6.1 Hz, 1H), 3.40 (t, J=5.9 Hz, 2H), 3.34 (s, 3H), 3.29 (dt, J=15.4, 5.3 Hz, 2H), 3.21 (q, J=5.8 Hz, 2H), 2.99 (dd, J=10.7, 9.2 Hz, 1H), 2.61 (s, 3H), 1.93-1.87 (m, 1H), 1.78-1.69 (m, 1H), 1.61-1.54 (m, 1H), 1.51-1.41 (m, 3H), 1.15 (ddd, J=23.3, 10.4, 3.5 Hz, 1H)

The compounds in Table 3 were prepared following procedures analogous to those described above, and optionally isolated as the designated salts.

The following are examples of aspartic protease inhibitors of the invention. When the stereochemistry at a chiral center is not defined in the compound name, this indicates that the sample prepared contained a mixture of isomers at this center.

TABLE 3

| Cpd. | | Mass |
|---|---|---|
| No. | Cpd Name | Observed |
| 1a | methyl {2-[((3-chlorophenyl){2-methyl-5-[({(2S)-2- | 532.5 |
| | (methylamino)-3-[(3R)-tetrahydro-2H-pyran-3-yl] | |
| | propyl}amino)carbonyl]phenyl} methyl)oxy]ethyl}carbamate | |
| 1b | methyl {2-[((S)-(3-chlorophenyl){2-methyl-5-[({(2S)-2- | 532.5 |
| | (methylamino)-3-[(3R)-tetrahydro-2H-pyran-3- | |
| | yl]propyl}amino)carbonyl]phenyl} methyl)oxy]ethyl}carbamate | |
| 2a | methyl {2-[((3-chlorophenyl){3-[({(2S)-2-(methylamino)-3- | 518.5 |
| | [(3R)-tetrahydro-2H-pyran-3-yl] | |
| | propyl}amino)carbonyl]phenyl} methyl)oxy]ethyl}carbamate | |
| 2b | methyl {2-[((R)-(3-chlorophenyl){3-[({(2S)-2-(methylamino)-3- | 518.0 |
| | [(3R)-tetrahydro-2H-pyran-3-yl]propyl} | |
| | amino)carbonyl]phenyl} methyl)oxy]ethyl}carbamate | |
| 2c | methyl {2-[((R)-(3-chlorophenyl){3-[({(2R)-2-(methylamino)-3- | 518.0 |
| | [(3S)-tetrahydro-2H-pyran-3-yl] | |
| | propyl}amino)carbonyl]phenyl} methyl)oxy]ethyl}carbamate | |
| 2d | methyl {2-[((R)-(3-chlorophenyl){3-[({(2S)-2-(methylamino)-3- | 518.0 |
| | [(3S)-tetrahydro-2H-pyran-3-yl] | |
| | propyl}amino)carbonyl]phenyl} methyl)oxy]ethyl}carbamate | |
| 2e | methyl {2-[((R)-(3-chlorophenyl){3-[({(2R)-2-(methylamino)-3- | 518.5 |
| | [(3R)-tetrahydro-2H-pyran-3-yl] | |
| | propyl}amino)carbonyl]phenyl} methyl)oxy]ethyl}carbamate | |
| 3a | methyl [2-({(3-chlorophenyl)[3-({[(2S)-3-cyclohexyl-2- | 516.7 |
| | (methylamino)propyl]amino} | |
| | carbonyl)phenyl]methyl}oxy)ethyl] carbamate | |
| 4a | methyl [2-({(3-chlorophenyl)[3-({[(2S)-4-methyl-2- | 476.4 |
| | (methylamino) pentyl]amino}carbonyl)phenyl] | |
| | methyl}oxy)ethyl]carbamate | |
| 5a | methyl [2-({(3-chlorophenyl)[3-({[(2S)-3-cyclohexyl-2- | 533.8 |
| | (methylamino)propyl]amino} carbonyl)-4-fluorophenyl]methyl} | |
| | oxy)ethyl] carbamate | |
| 6a | methyl {2-[((3-chlorophenyl){4-fluoro-3-[({(2S)-2- | 535.9 |
| | (methylamino)-3-[(3R)-tetrahydro-2H-pyran-3-yl] | |
| | propyl}amino)carbonyl]phenyl} methyl)oxy]ethyl}carbamate | |
| 7a | methyl [2-({(3-chlorophenyl)[5-({[(2S)-3-cyclohexyl-2- | 530.5 |
| | (methylamino)propyl]amino} carbonyl)-2- | |
| | methylphenyl]methyl} oxy)ethyl] carbamate | |
| 8a | methyl (2-{[{3-chloro-5-[({(2S)-2-(methylamino)-3-[(3R)- | 552.5 |
| | tetrahydro-2H-pyran-3-yl]propyl}amino) carbonyl] phenyl}(3- | |
| | chlorophenyl) methyl]oxy}ethyl)carbamate | |
| 9a | methyl (2-{[[3-chloro-5-({[(2S)-3-cyclohexyl-2- | 550.6 |
| | (methylamino)propyl] amino}carbonyl)phenyl](3- | |
| | chlorophenyl)methyl]oxy}ethyl) carbamate | |
| 10a | methyl [2-({(3-chlorophenyl)[5-({[(2S)-3-cyclohexyl-2- | 534.5 |
| | (methylamino)propyl]amino}carbonyl)-2-fluorophenyl]methyl} | |
| | oxy)ethyl] carbamate | |
| 11a | methyl [2-({(3-chlorophenyl)[3-({[(2S)-2-(methylamino)-3- | 518.5 |
| | (tetrahydro-2H-pyran-4-yl)propyl]amino}carbonyl)phenyl] | |
| | methyl}oxy)ethyl]carbamate | |
| 12a | methyl {2-[((3-chlorophenyl){2-fluoro-5-[({(2S)-2- | 536.4 |
| | (methylamino)-3-[(3R)-tetrahydro-2H-pyran-3- | |
| | yl]propyl}amino)carbonyl]phenyl} methyl)oxy]ethyl}carbamate | |
| 13a | methyl {2-[((5-chloro-2-methylphenyl){3-[({(2S)-2- | 532.3 |
| | (methylamino)-3-[(3R)-tetrahydro-2H-pyran-3- | |
| | yl]propyl}amino) carbonyl] phenyl}methyl)oxy] | |
| | ethyl}carbamate | |
| 13b | methyl {2-[((R)-(5-chloro-2-methylphenyl){3-[({(2S)-2- | 532.0 |
| | (methylamino)-3-[(3R)-tetrahydro-2H-pyran-3- | |
| | yl]propyl}amino) carbonyl]phenyl}methyl)oxy] | |
| | ethyl}carbamate | |

### Example 2

### In Vitro Activity Studies

The potency of renin inhibitors is measured using an in vitro renin assay. In this assay, renin-catalyzed proteolysis of a fluorescently labeled peptide converts the peptide from a weakly fluorescent to a strongly fluorescent molecule. The following test protocol is used. Substrate solution (5 ul; 2 uM Arg-Glu-Lys(5-Fam)-Ile-His-Pro-Phe-His-Leu-Val-Ile-His-Thr-Lys(5,6 Tamra)-Arg-CONH₂ in 50 mM Hepes, 125 mM NaCl, 0.1% CHAPS, pH 7.4) then trypsin-activated recombinant human renin (Scott, Martin J. et. al. Protein Expression and Purification 2007, 52(1), 104-116; 5 uL; 600 pM renin in 50 mM Hepes, 125 mM NaCl, 0.1% CHAPS, pH 7.4) are added sequentially to a black Greiner low volume 384-well plate (cat. #784076) pre-stamped with a 100 n1 DMSO solution of compound at the desired concentration. The assay plates are incubated at room temperature for 2 hours with a cover plate then quenched by the addition of a stop solution (2 uL; 5 uM of Bachem C-3195 in 50 mM Hepes, 125 mM NaCl, 0.1% CHAPS, pH 7.4, 10% DMSO). The assay plates are read on an LJL Acquest using a 485 nm excitation filter, a 530 nm emission filter, and a 505 nm dichroic filter. Compounds are initially prepared in neat DMSO at a concentration of 10 mM. For inhibition curves, compounds were diluted using a three fold serial dilution and tested at 11 concentrations (e.g. 50 μM-0.8 nM or 25 μM-0.42 nM or 2.5 μM to 42 pM). Curves were analyzed using ActivityBase and XLfit, and results were expressed as pIC₅₀ values.

The above-described in vitro enzyme activity studies were conducted for the compounds in Tables 2 and 3. Each of the compounds demonstrated an in vitro IC₅₀ of less than 1000 nM.

### Example 3

### In Vivo Activity Studies

The cardiac and systemic hemodynamic efficacy of renin inhibitors can be evaluated in vivo in sodium-depleted, normotensive cynomolgus monkeys. Arterial blood pressure is monitored by telemetry in freely, moving, conscious animals.

The efficacy of the renin inhibitors can also be evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Cynomolgus Monkey (General Method): Six male naïve cynomolgus monkeys weighing between 2.5 and 3.5 kg are to be used in the studies. At least 4 weeks before the experiment, the monkeys are anesthetized with ketamine hydrochloride (15 mg/kg, i.m.) and xylazine hydrochloride (0.7 mg/kg, i.m.), and are implanted into the abdominal cavity with a transmitter (Model #TL11M2-D70-PCT, Data Sciences, St. Paul, Minn.). The pressure catheter is inserted into the lower abdominal aorta via the femoral artery. The bipotential leads are placed in Lead II configuration. The animals are housed under constant temperature (19-25° C.), humidity (>40%) and lighting conditions (12 h light and dark cycle), are fed once daily, and are allowed free access to water. The animals are sodium depleted by placing them on a low sodium diet (0.026%, Expanded Primate Diet 829552 MP-VENaCl (P), Special Diet Services, Ltd., UK) 7 days before the experiment and furosemide (3 mg/kg, intramuscularly i.m., Aventis Pharmaceuticals) is administered at −40 h and −16 h prior to administration of test compound.

For oral dosing, the renin inhibitors are formulated in 0.5% methylcellulose at dose levels of 10 and 30 mg/kg (5 mL/kg) by infant feeding tubes. For intravenous delivery, a silastic catheter is implanted into posterior vena cava via a femoral vein. The catheter is attached to the delivery pump via a tether system and a swivel joint. Test compound (dose levels of 0.1 to 10 mg/kg, formulated at 5% dextrose) is administered by continuous infusion (1.67 mL/kg/h) or by bolus injection (3.33 mL/kg in 2 min).

Arterial blood pressures (systolic, diastolic and mean) and body temperature are recorded continuously at 500 Hz and 50 Hz, respectively, using the Dataquest™ A.R.T. (Advanced Research Technology) software. Heart rate is derived from the phasic blood pressure tracing. During the recording period, the monkeys are kept in a separate room without human presence to avoid pressure changes secondary to stress. All data are expressed as mean±SEM. Effects of the renin inhibitors on blood pressure are assessed by ANOVA, taking into account the factors dose and time compared with the vehicle group.

Double Transgenic Rats (General Method): Experiments are conducted in 6-week-old double transgenic rats (dTGRs). This model is described in the reference provided above. Briefly, the human renin construct that is used to generate transgenic animals made up the entire genomic human renin gene (10 exons and 9 introns), with 3.0 kB of the 5′-promoter region and 1.2 kB of 3′ additional sequences. The human angiotensinogen construct makes up the entire human angiotensinogen gene (5 exons and 4 introns), with 1.3 kB of 5′-flanking and 2.4 kB of 3′-flanking sequences. The rats may be purchased from RCC Ltd (Füllinsdorf, Switzerland). Radio telemetry transmitters are surgically implanted at 4 weeks of age. The telemetry system provides 24-h recordings of systolic, mean, diastolic arterial pressure (SAP, MAP, DAP, respectively) and heart rate (HR). Beginning on day 42, animals are transferred to telemetry cages. A 24 h telemetry reading is obtained. Rats are then dosed orally on the following 4 consecutive days (days 43-46). The rats are monitored continuously and are allowed free access to standard 0.3%-sodium rat chow and drinking water.

While this invention has been particularly shown and described with references to specific embodiments thereof, it will be understood by those skilled in the art that various changes in form and details may be made therein without departing from the scope of the invention encompassed by the appended claims.

1. A compound represented by the following Formula: 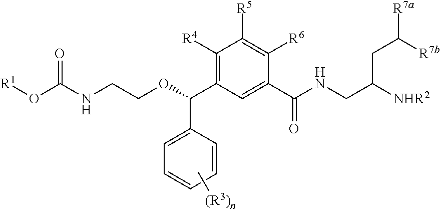
wherein:
R¹ is C₁-C₄ alkyl, C₃-C₆ cycloalkyl or C₃-C₆ cycloalkyl-C₁-C₄ alkyl-;
R² is H or C₁-C₄ alkyl;
each R³ is independently selected from F, Cl, Br, cyano, nitro, C₁-C₄ alkyl, C₁-C₄ haloalkyl, C₁-C₄ alkoxy, C₁-C₄ haloalkoxy, and C₁-C₄ alkylsulfonyl-;
n is 0, 1, 2, or 3;
R⁴, R⁵ and R⁶ are selected from H, halo and C₁-C₃ alkyl, wherein one of R⁴, R⁵ or R⁶ is H, halo or C₁-C₃ alkyl and the other two of R⁴, R⁵ and R⁶ are H; and
R^{7a} and R^{7b} are each independently C₁-C₃ alkyl, or R^{7a} and R^{7b} taken together with the carbon atom to which they are attached form a 5-6 membered carbocyclic or heterocylic ring, where the heterocyclic ring contains one oxygen atom;
or a pharmaceutically acceptable salt thereof.

2. The compound according to claim 1, wherein the compound is represented by the following Formula: 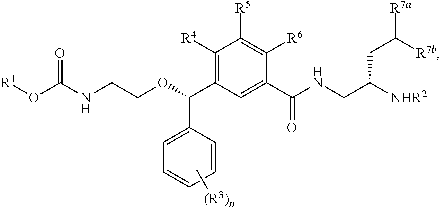
or a pharmaceutically acceptable salt thereof.

3. The compound according to claim 1, wherein R^{7a} and R^{7b} are each independently C₁-C₃ alkyl, or R^{7a} and R^{7b} taken together with the carbon atom to which they are attached form a cyclohexyl or a tetrahydropyranyl ring, or a pharmaceutically acceptable salt thereof.

4. The compound according to claim 1, wherein R^{7a} and R^{7b} taken together with the carbon atom to which they are attached form a cyclohexyl or a tetrahydropyranyl ring, or a pharmaceutically acceptable salt thereof.

5. The compound according to claim 1, wherein the compound is represented by the following Formula: 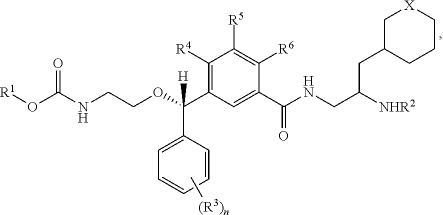
wherein X is CH₂ or O; or a pharmaceutically acceptable salt thereof.

6. The compound according to claim 1, wherein the compound is represented by the following Formula: 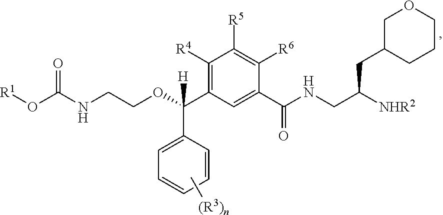
or a pharmaceutically acceptable salt thereof.

7. The compound according to claim 1, wherein the compound is represented by the following Formula: 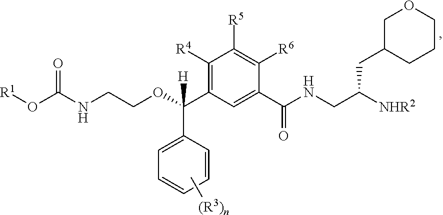
or a pharmaceutically acceptable salt thereof.

8. The compound according to claim 1, wherein R¹ is C₁-C₃ alkyl.

9. The compound according to claim 1, wherein R² is H or C₁-C₃ alkyl.

10. The compound according to claim 1, wherein R⁴, R⁵ and R⁶ are selected from H, F, Cl and C₁-C₃ alkyl, wherein one of R⁴, R⁵ or R⁶ is H, F, C₁ or C₁-C₃ alkyl and the other two of R⁴, R⁵ and R⁶ are H.

11. The compound according to claim 1, wherein n is 0, 1 or 2.

12. The compound according to claim 1, wherein R⁴, R⁵ and R⁶ are each H or one of R⁴, R⁵ or R⁶ is F, Cl or methyl.

13. The compound according to claim 1, wherein R¹ is methyl.

14. The compound according to claim 1, wherein R² is H or methyl.

15. The compound according to claim 1, wherein each R³ is independently selected from F, Cl, and methyl.

16. The compound according to claim 1, wherein n is 1.

17. The compound according to claim 1, wherein n is 2.

18. The compound according to claim 1, wherein R³ is F and n is 1.

19. The compound according to claim 1, wherein R³ is Cl and n is 1.

20. The compound according to claim 1, wherein n is 2, one R³ is Cl and the other R³ is methyl.

21. The compound according to claim 1, wherein n is 0.

22. The compound according to claim 1, wherein R⁴, R⁵ and R⁶ are each H.

23. The compound according to claim 1, wherein one of R⁴, R⁵ or R⁶ is F, Cl or methyl.

24. The compound according to claim 1, wherein R¹ is C₁-C₃ alkyl; R² is H or C₁-C₃ alkyl; each R³ is independently selected from F, Cl, cyano, nitro, C₁-C₃ alkyl, C₁-C₃ haloalkyl, C₁-C₃ alkoxy, C₁-C₃ haloalkoxy, and C₁-C₃ alkylsulfonyl-; n is 0, 1, or 2; and R⁴, R⁵ and R⁶ are selected from H, F, Cl and C₁-C₃ alkyl, wherein one of R⁴, R⁵ or R⁶ is H, F, C₁ or C₁-C₃ alkyl and the other two of R⁴, R⁵ and R⁶ are H; or a pharmaceutically acceptable salt thereof.

25. The compound according to claim 1, wherein R¹ is C₁-C₃ alkyl; R² is C₁-C₃ alkyl; each R³ is independently selected from F, Cl and C₁-C₃ alkyl; n is 0, 1 or 2; and R⁴, R⁵ and R⁶ are each H or one of R⁴, R⁵ or R⁶ is F, Cl or methyl; or a pharmaceutically acceptable salt thereof.

26. A compound selected from:
methyl {2-[((3-chlorophenyl){2-methyl-5-[({2-(methylamino)-3-[(3R)-tetrahydro-2H-pyran-3-yl]propyl}amino)carbonyl]phenyl}methyl)oxy]ethyl}carbamate;
methyl {2-[((3-chlorophenyl) {3-[({2-(methylamino)-3-[(3R)-tetrahydro-2H-pyran-3-yl]propyl}amino)carbonyl]phenyl}methyl)oxy]ethyl}carbamate;
methyl [2-({(3-chlorophenyl) [3-({[(2S)-3-cyclohexyl-2-(methylamino)propyl]amino}carbonyl)phenyl]methyl}oxy)ethyl]carbamate;
methyl [2-({(3-chlorophenyl)[3-({[(2S)-4-methyl-2-(methylamino)pentyl]amino}carbonyl)phenyl]methyl}oxy)ethyl]carbamate;
methyl [2-({(3-chlorophenyl) [3-({[(2S)-3-cyclohexyl-2-(methylamino)propyl]amino}carbonyl)-4-fluorophenyl]methyl}oxy)ethyl]carbamate;
methyl {2-[((3-chlorophenyl){4-fluoro-3-[({2-(methylamino)-3-[(3R)-tetrahydro-2H-pyran-3-yl]propyl}amino)carbonyl]phenyl}methyl)oxy]ethyl}carbamate;
methyl [2-({(3-chlorophenyl) [5-({[(2S)-3-cyclohexyl-2-(methylamino)propyl]amino}carbonyl)-2-methylphenyl]methyl}oxy)ethyl]carbamate;
methyl (2-{[{3-chloro-5-[({2-(methylamino)-3-[(3R)-tetrahydro-2H-pyran-3-yl]propyl}amino)carbonyl]phenyl}(3-chlorophenyl)methyl]oxy}ethyl)carbamate;
methyl (2-{[[3-chloro-5-({[(2S)-3-cyclohexyl-2-(methylamino)propyl]amino}carbonyl)phenyl] (3-chlorophenyl)methyl]oxy}ethyl)carbamate;
methyl [2-({(3-chlorophenyl)[5-({[(2S)-3-cyclohexyl-2-(methylamino)propyl]amino}carbonyl)-2-fluorophenyl]methyl}oxy)ethyl]carbamate;
methyl {2-[((R)-(3-chlorophenyl) {2-methyl-5-[({(2S)-2-(methylamino)-3-[(3R)-tetrahydro-2H-pyran-3-yl]propyl}amino)carbonyl]phenyl}methyl)oxy]ethyl}carbamate;
methyl [2-({(3-chlorophenyl)[3-({[(2S)-2-(methylamino)-3-(tetrahydro-2H-pyran-4-yl)propyl]amino}carbonyl)phenyl]methyl}oxy)ethyl]carbamate;
methyl {2-[((3-chlorophenyl){2-fluoro-5-[({(2S)-2-(methylamino)-3-[(3R)-tetrahydro-2H-pyran-3-yl]propyl}amino)carbonyl]phenyl}methyl)oxy]ethyl}carbamate;
methyl {2-[((S)-(3-chlorophenyl) {2-methyl-5-[({(2S)-2-(methylamino)-3-[(3R)-tetrahydro-2H-pyran-3-yl]propyl}amino)carbonyl]phenyl}methyl)oxy]ethyl}carbamate; and
methyl {2-[((R)-(3-chlorophenyl) {3-[({(2S)-2-(methylamino)-3-[(3R)-tetrahydro-2H-pyran-3-yl]propyl}amino)carbonyl]phenyl}methyl)oxy]ethyl}carbamate;
or a pharmaceutically acceptable salt thereof.

27. A compound selected from: 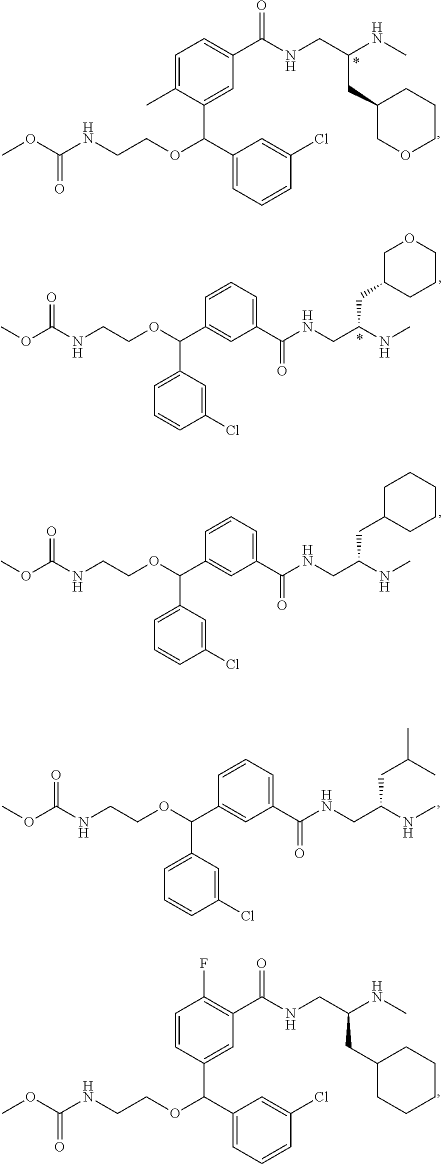 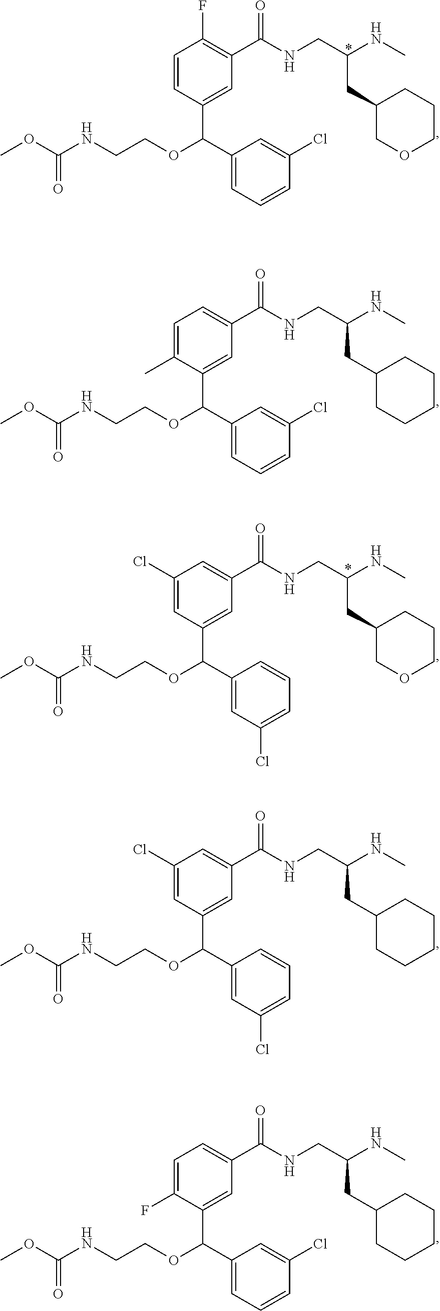 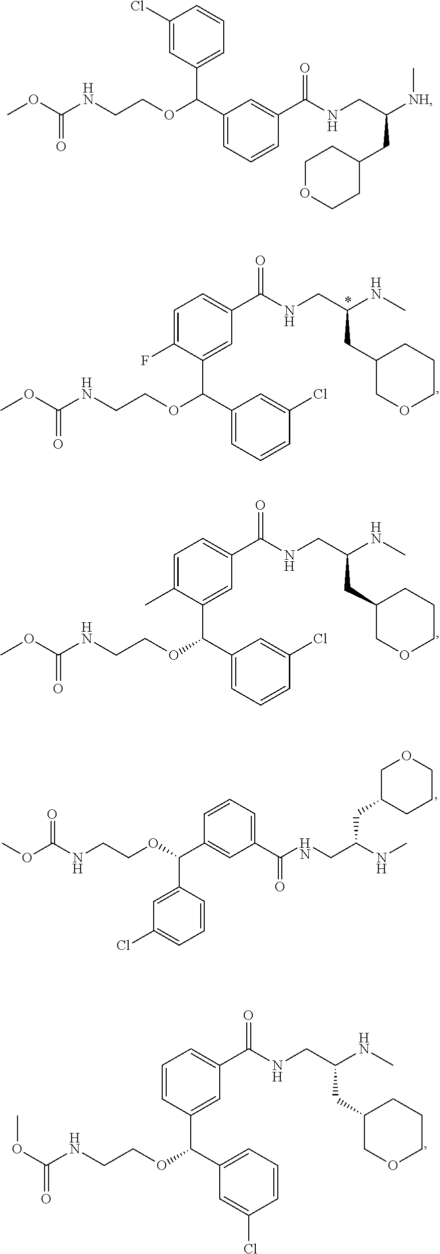 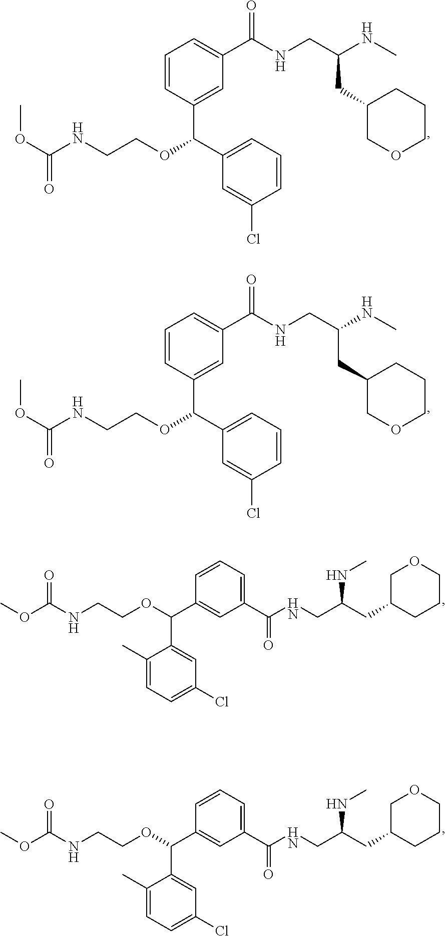
or a pharmaceutically acceptable salt thereof.

28. A pharmaceutical composition comprising a pharmaceutically acceptable carrier or diluent and the compound according to claim 1.

29. The pharmaceutical composition according to claim 28, further comprising a α-blocker, β-blocker, calcium channel blocker, diuretic, natriuretic, saluretic, centrally acting antiphypertensive, angiotensin converting enzyme inhibitor, dual angiotensin converting enzyme and neutral endopeptidase inhibitor, an angiotensin-receptor blocker, dual angiotensin-receptor blocker and endothelin receptor antagonist, aldosterone synthase inhibitor, aldosterone-receptor antagonist, or endothelin receptor antagonist.

30. A method of antagonizing one or more aspartic proteases in a subject in need thereof, comprising administering to the subject an effective amount of the compound according to claim 1.

31. The method according to claim 30, wherein the aspartic protease is renin.

32. A method for treating an aspartic protease mediated disorder in a subject comprising administering to the subject an effective amount of the compound according to claim 1.

33. The method according to claim 32, wherein said disorder is hypertension, congestive heart failure, cardiac hypertrophy, cardiac fibrosis, cardiomyopathy post-infarction, nephropathy, vasculopathy and neuropathy, a disease of the coronary vessels, post-surgical hypertension, restenosis following angioplasty, raised intra-ocular pressure, glaucoma, abnormal vascular growth, hyperaldosteronism, an anxiety state, or a cognitive disorder.

34. The method according to claim 32, further comprising administering one or more additional agents selected from the group consisting of an α-blockers, a β-blocker, a calcium channel blocker, a diuretic, an angiotensin converting enzyme inhibitor, a dual angiotensin converting enzyme and neutral endopeptidase inhibitor, an angiotensin-receptor blocker, dual angiotensin-receptor blocker and endothelin receptor antagonist, a aldosterone synthase inhibitor, a aldosterone-receptor antagonist, and an endothelin receptor antagonist.

35. The method according to claim 32, wherein the aspartic protease is β-secretase.

36. The method according to claim 32, wherein the aspartic protease is plasmepsin.

37. The method according to claim 32, wherein the aspartic protease is HIV protease.
